(19) United States
(12) Patent Application Publication
(10) Pub. No.: US 2020/0152298 A1
(43) Pub. Date: May 14, 2020

(54) BODY MANAGEMENT SYSTEM

(71) Applicant: Stephen Eisenmann, Vero Beach, FL (US)

(72) Inventor: Stephen Eisenmann, Vero Beach, FL (US)

(21) Appl. No.: 16/672,710

(22) Filed: Nov. 4, 2019

Related U.S. Application Data
(60) Provisional application No. 62/757,184, filed on Nov. 8, 2018.

(51) Int. Cl. G16H 10/60; G06Q 30/02; G16H 20/60; G16H 15/00; G16H 20/10; G16H 20/30; G16H 40/63; G16H 50/30; G06N 20/00; G06K 9/46; A61B 5/00; A61B 5/0205; A61B 5/11; A61B 5/145

(52) U.S. Cl. CPC G16H 10/60; A61B 5/021; G16H 20/60; G16H 15/00; G16H 20/10; G16H 20/30; G16H 40/63; G16H 50/30; G06N 20/00; G06K 9/46; A61B 5/486; A61B 5/4866; A61B 5/4833; A61B 5/746; A61B 5/0205; A61B 5/1118; A61B 5/14532; G06K 2209/17; G06Q 30/0224

(57) ABSTRACT

A computing system, device, and/or method with utilizes one or more processors which receive at least a first image and a second image, the one or more processors may determine a starting calories content of a first consumable item from extracting data from the first image and an ending calories content of the first consumable item from extracting data from the second image, the one or more processors may determine a first consumed calories by calculating a difference between the starting calories content of the first consumable item and the ending calories content of the first consumable item.

The present application claims priority to U.S. provisional patent application Ser. No. 62/757,184, filed on Nov. 8, 2018, which is incorporated in its entirety herein by reference.

### BACKGROUND

### Field

This disclosure relates to systems, devices, and methods which monitor, track, generate reports, generate warnings, and/or provide action plans relating to food intake, medicine intake, monitors body signals, and/or exercise completed to improve and/or enhance a person's health. Further, this disclosure relates to achieving and maintaining a healthy balance between food intake, medicine intake, exercise, normal bodily functioning (e.g., no external forces on the body which requires the body to work harder), and period where the body's functions are under external forces (e.g., periods where the body acting as a heat source/heat sink must act to maintain normal bodily conditions (e.g., 98.6 F on average).

### Description of the Related Art

Approximately 45 million people in the USA go on a diet each year and spend more than $30 billion dollars on weight loss products a year. Even with this huge expenditure of monies, it is estimated that over 65 percent of Americans are overweight. The cost in health risk, standard of living, and mental well-being is enormous for Americans. This disclosure aims to help reduce this problem.

### BRIEF DESCRIPTION OF THE DRAWINGS

The disclosure may be understood by reference to the following description taken in conjunction with the accompanying drawings, in which like reference numerals identify like elements, and in which:

FIG. 1 is an illustration of a calories intake process, according to one embodiment.

FIG. 2 is an illustration of a process flow chart for the calories intake process, according to one embodiment.

FIG. 3 is another illustration of a calories intake process, according to one embodiment.

FIG. 4A is an illustration of a first step in a calories intake process, according to one embodiment.

FIG. 4B is an illustration of a calories tracking process, according to one embodiment.

FIG. 5A is an illustration of a first step in a calories intake process, according to one embodiment.

FIG. 5B is an illustration of a calories tracking process, according to one embodiment.

FIG. 6A is an illustration of a first step in a calories intake process, according to one embodiment.

FIG. 6B is an illustration of a calories tracking process, according to one embodiment.

FIG. 7 is a block diagram, according to one embodiment.

FIG. 8 is an illustration of a calorie intake and tracking graphical report, according to one embodiment.

FIG. 9 is an illustration of a calorie intake, calorie utilization, and tracking graphical report, according to one embodiment.

FIG. 10 is an illustration of a calorie utilization tracking process, according to one embodiment.

FIG. 11 is an illustration of a weight program process, according to one embodiment.

FIG. 12 is an illustration of a flow chart for a calorie and medicine intake procedure along with an exercise tracker and body signals monitoring procedures, according to one embodiment.

FIG. 13 is an illustration of a shirt with a heat exchanging system, according to one embodiment.

FIG. 14A is an illustration of the front side of the disclosed shirt with a heat exchanging system, according to one embodiment.

FIG. 14B is an illustration of the back side of the disclosed shirt with a heat exchanging system, according to one embodiment.

FIG. 15 is an illustration of a heat exchanging system, one or more processors, one or more sensors, and a controller, according to one embodiment.

FIG. 16 is an illustration of a block diagram, according to one embodiment.

FIG. 17 is an illustration of a controller, according to one embodiment.

FIG. 18 is a process flow, according to one embodiment.

FIG. 19 is an illustration of Peltier based method, system, and/or device, according to various embodiments.

While the disclosure is susceptible to various modifications and alternative forms, specific embodiments thereof have been shown by way of example in the drawings and are herein described in detail. It should be understood, however, that the description herein of specific embodiments is not intended to limit the disclosure to the particular forms disclosed, but on the contrary, the intention is to cover all modifications, equivalents, and alternatives falling within the spirit and scope of the disclosure as defined by the appended claims.

### DETAILED DESCRIPTION OF SPECIFIC EMBODIMENTS

Illustrative embodiments of the disclosure are described herein. In the interest of brevity and clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

This document does not intend to distinguish between components that differ in name but not function. In the following discussion and in the claims, the terms “including” and “includes” are used in an open-ended fashion, and thus should be interpreted to mean “including, but not limited to.” Also, the term “couple” or “couples” is intended to mean either a direct or an indirect connection (e.g., electrical, mechanical, etc.). “Direct contact,” “direct attachment,” or providing a “direct coupling” indicates that a surface of a first element contacts the surface of a second element with no substantial attenuating medium there between. The presence of small quantities of substances, such as bodily fluids, that do not substantially attenuate electrical connections does not vitiate direct contact. The word “or” is used in the inclusive sense (i.e., “and/or”) unless a specific use to the contrary is explicitly stated.

The particular embodiments disclosed above are illustrative only as the disclosure may be modified and practiced in different but equivalent manners apparent to those skilled in the art having the benefit of the teachings herein. Furthermore, no limitations are intended to the details of construction or design herein shown other than as described in the claims below. It is, therefore, evident that the particular embodiments disclosed above may be altered or modified and all such variations are considered within the scope and spirit of the disclosure. Accordingly, the protection sought herein is as set forth in the claims below.

All locations, sizes, shapes, measurements, ratios, amounts, angles, component or part locations, configurations, dimensions, values, materials, orientations, etc. discussed or shown in the drawings are merely by way of example and are not considered limiting and other locations, sizes, shapes, measurements, ratios, amounts, angles, component or part locations, configurations, dimensions, values, materials, orientations, etc. can be chosen and used and all are considered within the scope of the disclosure.

Dimensions of certain parts as shown in the drawings may have been modified and/or exaggerated for the purpose of clarity of illustration and are not considered limiting.

The methods and/or methodologies described herein may be implemented by various means depending upon applications according to particular examples. For example, such methodologies may be implemented in hardware, firmware, software, or combinations thereof. In a hardware implementation, for example, a processing unit may be implemented within one or more application specific integrated circuits (“ASICs”), digital signal processors (“DSPs”), digital signal processing devices (“DSPDs”), programmable logic devices (“PLDs”), field programmable gate arrays (“FPGAs”), processors, controllers, micro-controllers, microprocessors, electronic devices, machine learning devices, smart phones, smart watches, other devices units designed to perform the functions described herein, or combinations thereof.

Some portions of the detailed description included herein are presented in terms of algorithms or symbolic representations of operations on binary digital signals stored within a memory of a specific apparatus or a special purpose computing device or platform. In the context of this particular specification, the term specific apparatus or the like includes a general purpose computer once it is programmed to perform particular operations pursuant to instructions from program software. Algorithmic descriptions or symbolic representations are examples of techniques used by those of ordinary skill in the arts to convey the substance of their work to others skilled in the art. An algorithm is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

Reference throughout this specification to “one example,” “an example,” “embodiment,” “another example,” “in addition,” “further,” and/or any similar language should be considered to mean that the particular features, structures, or characteristics may be combined in one or more examples. Any combination of any element in this disclosure with any other element in this disclosure is hereby disclosed.

While there has been illustrated and described what are presently considered to be example features, it will be understood by those skilled in the art that various other modifications may be made, and equivalents may be substituted, without departing from the disclosed subject matter. Additionally, many modifications may be made to adapt a particular situation to the teachings of the disclosed subject matter without departing from the central concept described herein. Therefore, it is intended that the disclosed subject matter not be limited to the particular examples disclosed.

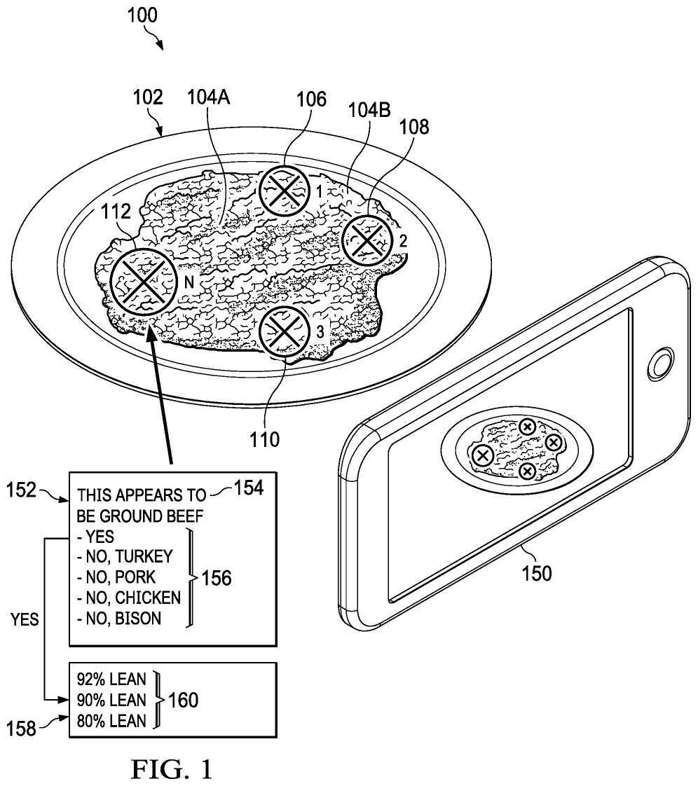

In FIG. 1, an illustration of a calories intake process is shown, according to one embodiment. In this example, a meal 100 includes various items including a first piece of pasta 104A, an Nth piece of pasta 104B, a first meatball 106, a second meatball 108, a third meatball 110, and an Nth meatball 112 where each item (e.g., pasta, sauce, meatball, tomato, etc.) has numerous different features (e.g., size, shape, color, density, calorie content, volume, etc.). Each item has a different size and a different calorie count. In this example, a picture (e.g., 2D picture, 3D picture, video, and/or a combination thereof) is taken of the meal 100 on a plate 102 with a camera 150 (and/or any device with a camera feature). In this example, the system, method, and/or device via one or more processors (and/or one or more engines) determines the calorie amount of each item on the plate 102 based on a calculation of size, diameter, volume, density, color, shape, etc. In one example, a diameter of a first meatball, a second meatball, and an Nth meatball are determined (based on the meatball having a shape of a ball). Based on this information and a determination that the meat is turkey (turkey with x density, x calories per unit, etc.), the system, method, and/or device determines, derives (obtain something from a source, to reach or obtain by reasoning, deduce, infer), and/or extracts (to draw from) from the picture that there are 800 calories of meatballs (e.g., Meatball 1=70 calories, meatball 2=50 calories, meatball 3 to Nth−1=620 calories, and meatball Nth=60 calories). In addition, these calculations can be learned over time, based on crowdsourced data, default settings, user default settings, user profiles, lookup table, a library, bar codes, brand names (e.g., Pepsi™), scanned words (ingredients equals flour, milk, etc.), voice commands (e.g., I ate an X candy bar), and/or any other food content identifier, menu picture or data, stored recipe, and/or any combination thereof. In one example, the default setting for a user may utilize 90% lean grounded beef for a picture that indicates that a meat is being utilized. A prompt can be generated to confirm this indication. In another example, the video may be utilized to determine how fast a person is eating or drinking a meal and/or a drink. For example, a person may eat a combo meal with 1200 calories in 5 minutes. The system, method, and/or device may recommend that the user eat the meal at a slow rate. In another example, the person using the video and/or picture may drink 4 beers in an hour. Based on the known body weight of the user, the system, method, and/or device may generate a warning that your blood alcohol level is above a predetermined threshold. The system, method, and/or device may have a GPS function which known where the person is located. Based on this information, the system, method, and/or device may utilize (via the Internet and/or stored data) the local laws relating to drinking and driving and/or be inebriated and initiate one or more actions. These actions could be a warning, calling a third party driving, disabling the user's car, communicating (e.g., phone, text, email) a third person, and/or generating a recommended course of action (e.g., you should drink water for the next two hours, etc.).

The person eating this food takes the picture before the consumption of any of the meal 100. This calculation yields the amount of calories available (e.g., 980 calories) to consume on the plate 102. In another example, a person may state “I am drinking 10 ounces of milk”. In another example, a person may state “I am drinking a 12 ounce Pepsi™”. In another example, a person may state “I am eating a quarter pounder with cheese from McDonalds™”. The system, method, and/or device may utilize the Internet to look up how many calories are in a standard quarter pounder with cheese from McDonalds™. In a first example, the camera 150 may take a 2D picture, a 3D picture, a video, and/or a combination thereof. The system, method, or device may use a 2D picture; a 3D picture, a video, and/or a combination thereof picture to derive the calorie amount present in the 2D picture, a 3D picture, a video, and/or a combination thereof.

In this example, one or more calorie determining engines may want to confirm that the meatballs are made out of ground beef. Therefore, the one or more calorie determining engines may request input from the user. In one example, the one or more calorie determining engines may generate a prompt 152 which states “This appears to be ground beef”. The user can then select yes as an answer indicating that the meatballs are made out of ground beef (e.g., cow). In another example, the one or more calorie determining engines may generate a second prompt 158 that presents the user with addition input options 160 to indicate the fat content of the ground beef. In this example, the options are a first option—92% lean (8% fat), a second option—90% (10% fat), and an Nth option 80% lean (20% fat). Further, if the meatballs are not made of ground beef from a cow, the user can select other meats (e.g., turkey, pork, chicken, bison, etc.) and/or other non-meats (e.g., beans, etc.) via a third prompt 156. In this example, the system, method, and/or device may determine the material of the examined food. In other words, the system, method, and/or device may determine that the meat is grounded beef without user input, and/or based on a default setting, and/or the user profile, and/or one or more characteristics derived from the picture (e.g., image, video, etc.). The system, method, and/or device may utilized this information and/or ask the user to confirm that the meat is grounded beef (e.g., 20 percent fat, 10 percent fat, etc.). In one example, the system, device, and/or method may determine that a first meatball has a sphere form plus or minus 1 percent (any percentage error can be used from 0.1 to 100 percent and are all within the scope of ranges which can be used as limitations for the claims—0.1 to 0.5, 1 to 2, 4 to 7, etc.—for brevity they are not all listed). In this example, the volume is calculated as V=4/3 πr³. The system, device, and/or method determines that the radius is ½ an inch. Therefore, the volume is 4/3 π(0.5)³. Based on this volume calculation and the calculated (and/or looked up) density of the 80 percent grounded beef (cow), the system, method, and/or device determines that the calories for this first meatball are 55 calories plus or minus 1 percent. In another example, a piece of spaghetti has a shape of a rectangle plus or minus 10 percent. In this example, the volume is calculated as V=length times width times height. The length is 1 inch, the width is 0.3 inches, and the height is 0.4 inches. Therefore, the volume can be calculated as 0.12 inches³. In this example, the system, method, and/or device determines that the calories for the spaghetti is 10 calories based on the calculated and/or stored density of spaghetti and the volume. Numerous but similar calculations are generated for the sauce and tomatoes to complete the total calories available for consumption in the meal 100. In various examples, the system, method, and/or device can use a volume calculation for a cube (e.g., V=S³), a volume calculation for a rectangular prism (e.g., V=lwh), a volume calculation for a cylinder (e.g., V=πr²h), a volume calculation for a regular square pyramid (e.g., V=⅓ bh), a volume calculation for a cone (e.g., V=⅓πr²h), a volume calculation for a sphere (e.g., V=4/3πr³), and/or any other volume equation, and/or any combination thereof to be utilized to determine a calorie amount. In various examples, one or more of these calculations may be required to determine the calorie content of an item (e.g., ice cream cone and ice cream, etc.). In various examples, a density and/or calories of an consumable item and/or calorie content may be determined via crowdsourced data, default settings, user default settings, user profiles, lookup table, a library, bar codes, brand names, scanned words, voice commands, and/or any other food content identifier, food composition database, calorie calculator, and/or any combination thereof.

In another example, a portion of a meatball (and/or the whole meatball) may have an irregular shape. In this example, the irregular shape may be broken down into Nth portion where a first portion has a first shape, a second portion has a second shape, and an Nth portion has a Nth shape. In this example, the system, method, and/or device may calculate the calorie content for each portion and add them together to obtain the calorie content for the portion of the meatball (and/or the whole meatball).

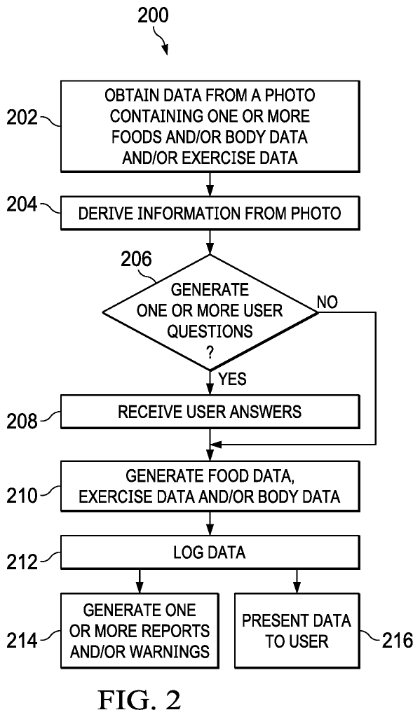

In FIG. 2, an illustration of a process flow chart for the calories intake process is shown, according to one embodiment. A method 200 may include obtaining data from a photo containing one or more foods and/or one or more medicines, and/or body data from one or more body data signals, and/or exercise data from one or more photos (and/or any other input) (step 202). The method 200 may include deriving information from the photo via one or more processors and/or one or more sensors, and/or one or more modules, and/or one or more engines (step 204). The method 200 may include generating one or more user questions (step 206). If user questions are to be utilized, then the method 200 receives user answers (step 208) and proceeds to step 210. If no user questions are to be utilized, then the method 200 proceeds to step 210. The method 200 may generate food data, medical data, exercise data, and/or body data (step 210). The method 200 may log one or more data points and/or calculate, derive, model, infer, and/or any other data processing function based on the data points—any function disclosed in this document can be utilized here (step 212). The method 200 may generate one or more reports, one or more recommendations, initiate one or more actions, and/or warnings (step 214). The method 200 may present data to the user (step 216).

Machine learning models relating to how these individuals consume food, consume medicine, generate body data, and/or exercise may be extrapolated and applied for benchmarking and/or baseline purposes. Further, cultural biases that exist may be identified and the system, method, and/or device may also determine whether they are to be used with the user profile.

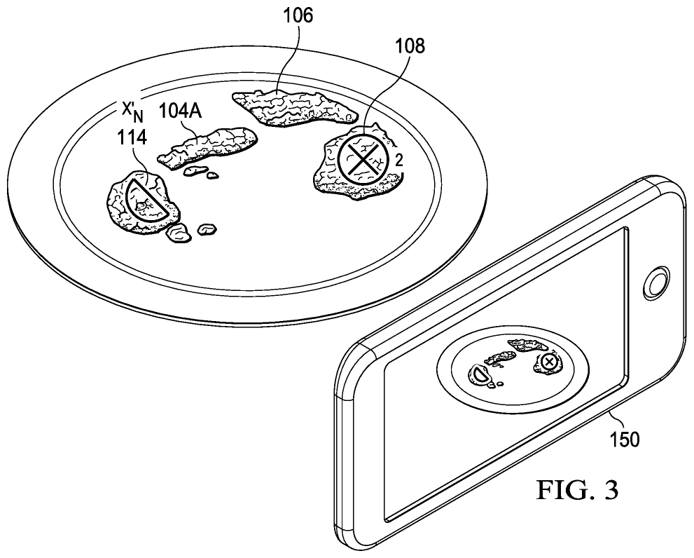

In FIG. 3, an illustration of a calories intake process is shown, according to one embodiment. In this example, the meal 100 from FIG. 1 has been partially consumed. The person takes a picture of the remaining food on the plate 102 with the camera 150 (and/or any device with a camera function). In this example, the system, method, and/or device via one or more processors (and/or one or more engines) determines the calorie amount of each item based on a calculation of size, volume, density, color, shape, etc. The person eating this food takes the picture after (end of eating meal) the consumption of any of the meal 100. This calculation yields the amount of calories available (e.g., 380 calories) to still be consumed on the plate 1402. Based on this information, the system, method, and/or device determines that 600 calories (980 calories available to eat from the first photo (and/or video) and 380 calories from the second photo (and/or video) remaining equals 600 calories consumed) were consumed by the individual. If there was no food on the plate 1402, then the amount of calories consumed would have been 980 calories. In another example, a video may be taking of a meal preparation (e.g., putting all the ingredients together and the system, method, and/or device can calculate the amount of total calories in the meal. The system, method, and/or device can calculate the amount of calories on each individual plate based on the calorie amount total for the entire prepared meal and/or calculate the calories based on the procedures described above.

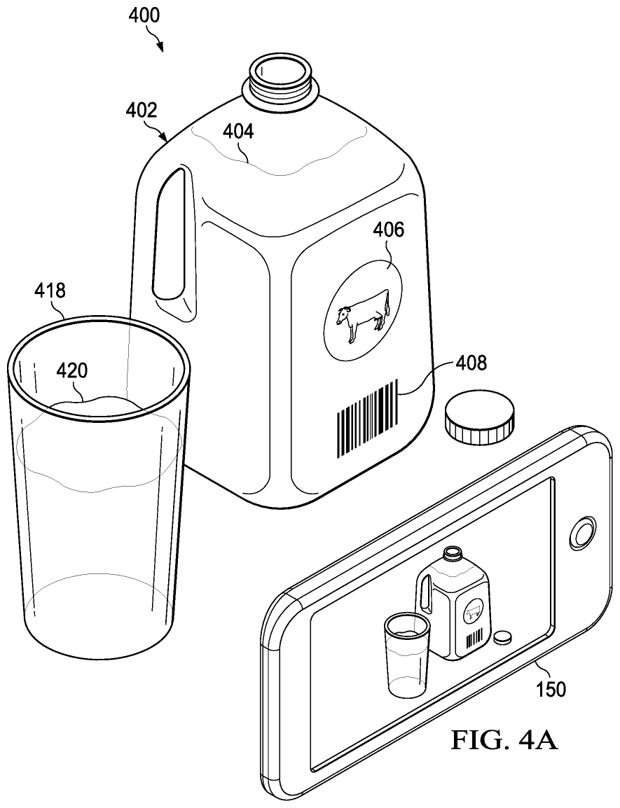

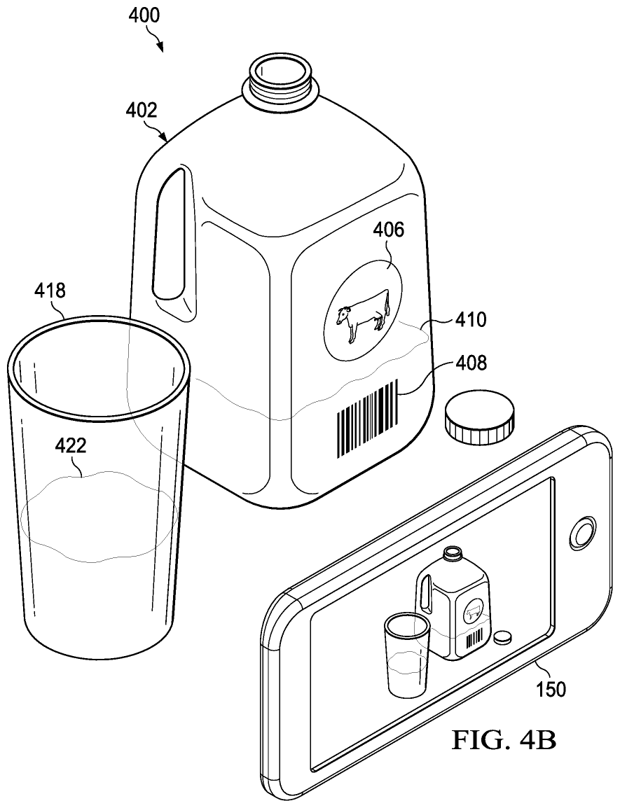

In FIG. 4A, an illustration of a calories intake process is shown, according to one embodiment. In this example, a first liquid 402 is shown in a bottle 400. The bottle 400 has a symbol 406, a bar code 408, and/or any other identifying data. The bottle is full at a first level 404. The person takes a picture of bottle 400 with the camera 150 before the person drinks the liquid inside the bottle 402. In this example, the system, method, and/or device determines the content of the bottle 400 via the symbol 406 (e.g. Mike, Coke, diet Coke, etc.), the bar code 408, and/or any other identifying data. In one example, the system, method, and/or device determines the amount of calories available (e.g., 2000 calories) in this bottle 400. The person then takes a picture of bottle 400 after consuming the first liquid 402 inside the bottle 400 to a finished level 410. The system, method, and/or device determines that the amount of calories available at the end of drinking was 500 calories. Therefore, the person consumed 1500 calories (75% of the total available calories) which is then logged and/or used for any purpose disclosed in this document. In various other examples, the amount of calories available in the bottle could be 300 calories (e.g., Pepsi) and the user would have 225 calories using the above example as a guide. In another example, Stevie Eisenmann (a first person—first user) wants to drink the milk out of a glass 418. Therefore, Stevie fills the glass 418 up to a first level 420. In one example, the system, method, and/or device determines the amount of calories available (e.g., 300 calories) in this glass 418 at the first level 420. Stevie then drinks the milk in the glass 418 to a second level 422 (see FIG. 4B). The system, method, and/or device determines the amount of calories available (e.g., 100 calories) in this glass 418 at the second level 422. Therefore, the system, method, and/or device determines that Stevie consumed 200 calories which is reported, logged, stored, and/or utilized for another calculation. In various examples, the system, method, and/or device may utilize different milk versions based on a first user (e.g., 2%), a second user (e.g., 1%), a third user (e.g., skim), and an Nth user (e.g., goat milk).

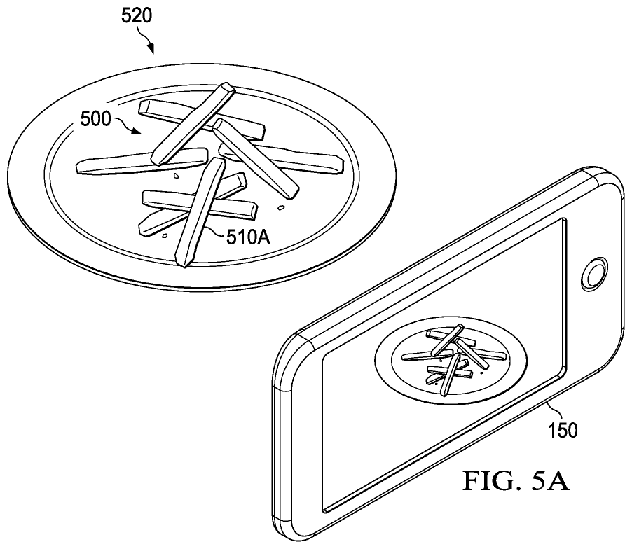

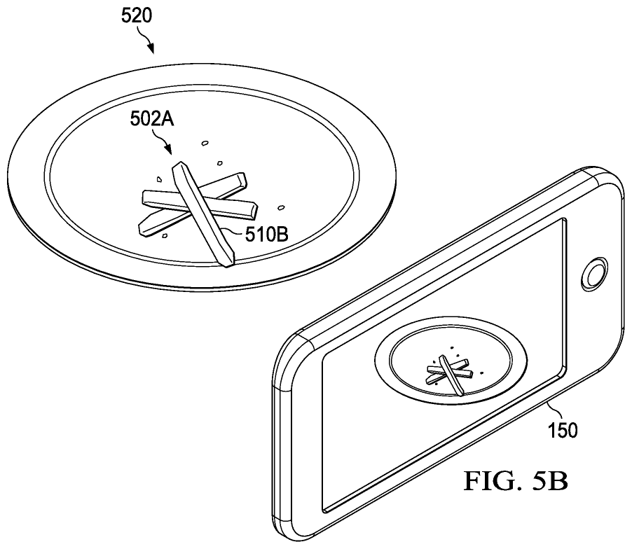

In FIGS. 5A and 5B, an illustration of a calories intake process is shown, according to one embodiment. In this example, Michael Eisenmann (a second person—a second user) takes a picture of a plate of fries 520 before consuming any fries 500 via camera 150 (e.g., camera, smart phone, smart watch, any camera like device, etc.). The system, method, and/or device determines that 500 calories are present based on a calculation of the shape, diameter, volume, color, density, etc. In addition, these calculations can be learned over time, based on crowdsourced data, default settings, user default settings, user profiles, bar codes, brand names, scanned words, and/or any other food content identifier, and/or any combination thereof. It should be noted that an Nth fry 510A is located in an Nth position. After Michael has finished eating the fries, Michael takes a second picture of the plate of a remaining fires 502A. The system, method, and/or device determines that 200 calories are present after the consumption of fries based on a second picture taken after the consumption of fries has ended—please note that the fries have moved and the system, method, and/or device still determines the correct amount of calories remaining. Therefore, the person has consumed 300 calories which is reported out, logged, and/or communicated in any other way and/or use for any other function described in this document. Further, one or more warnings might be generated based on the amount of calories consumed. For example, an exceeding daily target warning, exceeding meal calorie target warning, etc. In addition, motivational messages may be communicated. In this example, it should be noted that the Nth fry 510A has moved from an Nth position to an Nth prime position. The system, method, and/or device may be configured to identify a movement of a specific item. In another example, a picture and/or video may be taken of the floor (where food has been drop by a baby or others) to calculate the correct amount of calories consumed.

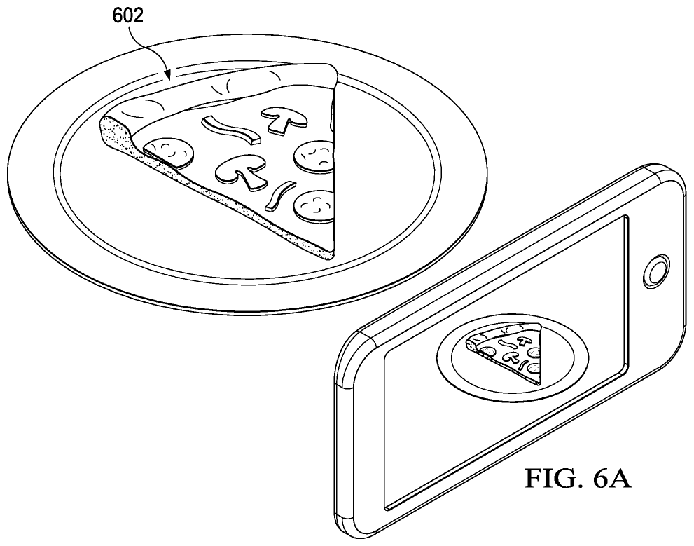

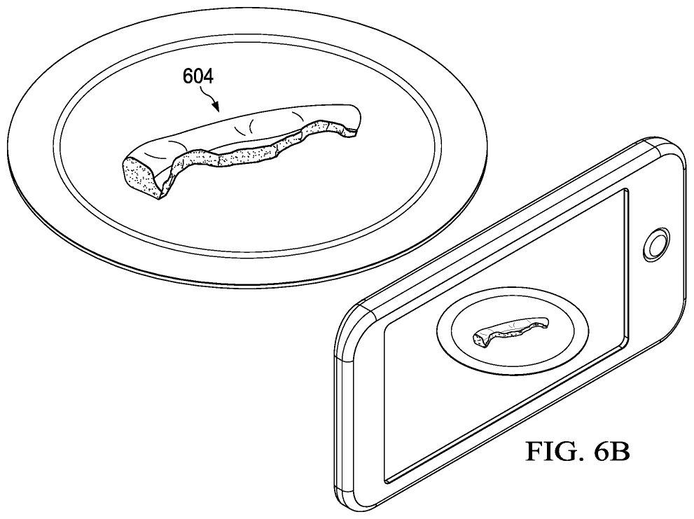

In FIGS. 6A and 6B, an illustration of a calories intake process is shown for a piece of pizza, according to one embodiment. In this example, Jordan Eisenmann (a third person—third user) takes a picture of a slice of pizza 602 before consuming the pizza via the camera 150. The system, method, and/or device determines that 400 calories are present based on a calculation of the shape, diameter, volume, color, density, etc. In addition, these calculations can be learned over time, based on crowdsourced data, default settings, user default settings, user profiles, bar codes, brand names, scanned words, and/or any other food content identifier, and/or any combination thereof. After Jordan consumes part of the pizza, Jordan takes a picture of a remaining pizza 604. The system, method, and/or device determines that 75 calories are present after the consumption of pizza based on a second picture taken after the consumption of the slice of pizza has ended. Therefore, the person has consumed 300 calories which is reported out, logged, and/or communicated in any other way and/or use for any other function described in this document. Further, one or more warnings might be generated based on the amount of calories consumed. For example, an exceeding daily target warning, exceeding meal calorie target warning, medicine warning, exercise note etc. may be generated. For example, you need 20 minutes on X machine to make up for eating X. In another example, a system default setting for pizza may be a cheese pizza but for Jordan her personal user default setting is a mushroom pizza.

In FIG. 700, a block diagram is shown, according to one embodiment. The system, device, and/or method may include and/or utilize one or more processors 702, one or more camera 704, one or more sensors 706, one or more modules 708, one or more engines 710, one or more tracking modules 712, one or more calorie intake modules 714, one or more reporting modules 716, one or more user profiles 718, and/or any other module and/or device described in this disclosure. Further, these listed items may communicate with one or more servers 720, one or more smart watches 722, one or more smart phones 724, one or more computers 732, one or more computer servers 728, one or more third party modules and/or engines 726, the internet 730, and/or each other. Further, the one or more servers 720, the one or more smart watches 722, the one or more smart phones 724, and/or the one or more computers 732 may communicate with each other.

In another example, Zenta Eisenmann (a fourth person—fourth user) has a cookie and takes a picture of the cookie. The system, method, and/or device determines the amount of calorie available for intake base on the size, diameter, shape, ingredients, color, and/or any other data. Further, a pop-up window (e.g., chocolate chip cookie, raisin cookie, oatmeal cookie, sugar cookie, etc.) may appear which states this is a cookie please select what type of cookie. In this example, 180 calories are available. The person then eats some of the cookie and takes a second picture of the partially eaten cookie. The system, method, and/or device determines that 90 calories are left to consume. Therefore, the person consumed 90 calories.

In another example, Katie Eisenmann (a fifth person—fifth user) has dinner at a sushi restaurant. Katie takes a first picture of the food before she eats some of the food—the system, method, and/or device determines the calorie content of the food via a 3D photo. Katie eats some of the food and then takes a second picture (3D) of the remaining food once she is done eating—the system, method, and/or device determines the calorie content which is left in the uneaten food and based on the calculations relating to the first photo and the second photo a consumed calories can be determined. Katie wants to complete a social media review (e.g., Yelp™) and/or post a photo onto social media (e.g., Facebook™). These functions can be completed with a simple push of a button, voice commands, and/or swiping function. In addition, one or more promotional offers (e.g., coupons, specials, etc.) may be generated by one or more third parties based on Katie eating sushi. In addition, the sushi restaurant may receive Katie's meal order electronically and be able to send one or more promotions based on Katie's meal order. For example, Katie you enjoyed meal 1, please come back for 10% off of meal 1. In another example, Katie you enjoyed meal 1, people who like meal 1 also like meal 2.

In another example of a calories tracking process, a report and/or graphical layout shows that the person had breakfast (e.g., 2 eggs, 1½ pieces of bacon, ¾ cup of orange juice which was 560 calories), lunch (e.g., a tuna fish sandwich which was 350 calories), but has not had dinner yet. In this example, the daily calorie target was 2000 calories. Therefore, the remaining calories for the day to reach the target is 1090 calories (e.g., 2000 calorie target minus 910 calories consumed=1090 calories). In one example, the system, method, and/or device may recommend one or more meal options based on the calories remaining until the daily calorie goal is reached.

In one example, the system, method, and/or device may generate a report based on a status of a person at a particular time. For example, Bob you are doing great you have only eaten 40% of your calorie goal and its 5 PM. In another example, Jan you have only consumed 50% of your calories, you are on your way to only eating 1000 calories today which will increase your weight loss to 2 pounds per month (week, etc.) instead of 1 pound per month (week, etc.). In another example, Marshal you have only consumed 10% of your calorie intake for carbohydrates—this is much better than your 20% goal limit. In another example, Charles you have eaten no vegetables today, please eat some if you get a chance.

In another example, Stephen Eisenmann buys 1 pound of roast beef every week. The system, method, and/or device may track how much roast beef is in each sandwich Stephen makes at home and send an indication when more roast beef should be order. For example, it may be automatically added to the electronic shopping list for Walmart™ or the system, device, and/or method may prompt Stephen to determine if more roast beef should be added. In various other examples, any food can be utilized (e.g., eggs, milk, soda, water, bread, etc.). Further, this information may be tied to store data (e.g., Walmart™). For example, in the example shown above with the roast beef for Stephen, the system, device, and/or method may be programmed to order a first roast beef (e.g., London broil) if it is on sale but if it is not on sale to order a second roast beef (e.g., baseline roast beef) or if the second roast beef is above a first cost to order a generic roast beef. In another example, the system, device, and/or method may rotate from a first meat (e.g., roast beef) to a second meat (e.g., turkey) and/or to an Nth meat (e.g., chicken). In addition, the system, method, and/or device may track the amount of food consumed and food purchased to generate various reports and/or any other function in this document. For example, a first report may show what food groups (proteins, carbohydrates, vegetables, treats, etc.) are being purchased and the cost associated with same. In another example, a second report may show what food has been wasted in what food groups and the cost associated with same. This waste information may be utilized for compost calculations (e.g., how much can you make). In addition, the system, method, and/or device may generate a report which shows that if you shopped at store X instead of store Y you could save a first amount.

In addition, these reports could be generated for an individual user. Stephen consumed (X % of vegetables, Y % of proteins, Z % of treats, and A % of carbohydrates, etc.) this food. The cost associated for this was $20 a day and Stephen wasted X amount of food (e.g., B % of vegetables, C % of proteins, D % of treats, and E % of carbohydrates, etc.) with an associated cost of $5 a day. The system, method, and/or device could also recommend diet changes. Stephen consumed (X % of vegetables, Y % of proteins, Z % of treats, and A % of carbohydrates, etc.) this food, however, Stephen would be healthier if Stephen consumed (X′% of vegetables, Y′% of proteins, Z′% of treats, and A′% of carbohydrates, etc.) of food. In another example, the system, method, and/or device could recommend a diet based on one or more medicines taken and/or one or more health conditions (e.g., age, disease (diabetes, cancer, etc.), professional athlete, etc.). Since you are on Medicine AA, you should consume X % of vegetables, Y % of proteins, Z % of treats, and A % of carbohydrates. Based on the foods you like in your user profile (and/or not on foods you like in your user profile), here is a recommended daily, weekly, and/or monthly consumption plan.

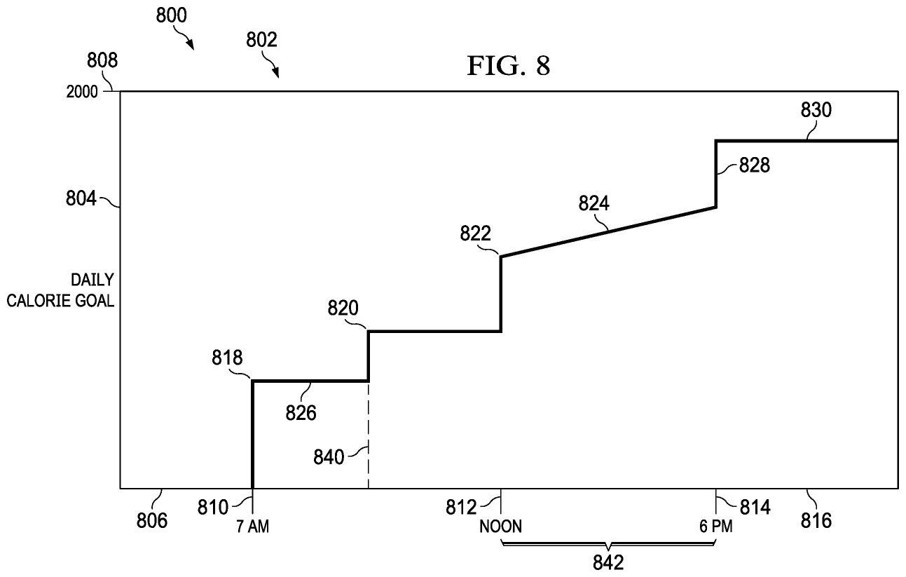

In FIG. 8, an illustration of a calorie intake and tracking graphical report 800 is shown, according to one embodiment. In this example, an X-Y chart with calories on the Y-Axis 804 and time on the X-Axis 806 is shown. In this example, a target daily calorie intake 808 is 2000 calories. In addition, the person has consumed a first meal 818 at a first time 810, a second meal 820 at a second time 840, a third meal 822 at a third time 812, a fourth meal 824 at a fourth time period 742, a fifth meal 828 at a fifth time 814. Please note that the meals look instantaneous just for graphical representation and did not occur instantaneous. Further, the fourth meal 2524 has a significant protraction of a period which causes the sloping line. This could be for example snacking for that period of time.

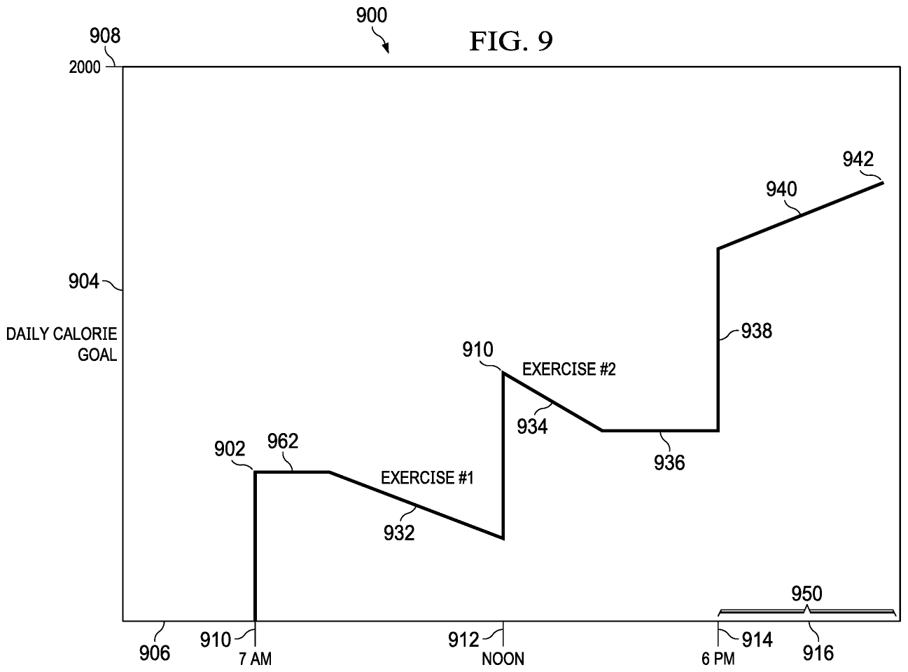

In FIG. 9, an illustration of a calorie intake, calorie utilization, and tracking graphical report is shown, according to one embodiment. The difference from FIG. 8 is that calories out (e.g., exercise) is taken into account. This can be seen in a first exercise 932 and an Nth exercise 934. In this example, an X-Y chart with calories on the Y-Axis 904 and time on the X-Axis 906 is shown. In this example, a target daily calorie intake 908 is 2000 calories. In addition, the person has consumed a first meal 902 at a first time 910, completed a first exercise 932, consumed a second meal 910 at a second time 912, completed a second exercise 934, consumed a third meal 938 at a third time 914, and a fourth meal 940 at a fourth time period 950. Please note that the meals look instantaneous just for graphical representation and did not occur instantaneous. Further, the fourth meal 940 has a significant protraction of a period which causes the sloping line. This could be for example snacking for that period of time. In addition, a first non-calorie period 962 and an Nth calorie period 936 are shown where no calories are consumed by the individual.

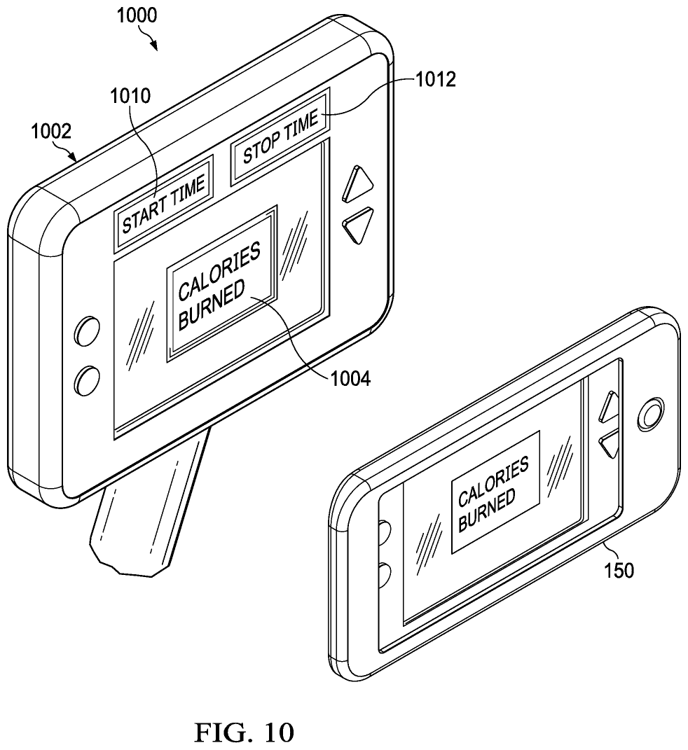

In FIG. 10, an illustration of a calorie utilization tracking process is shown, according to one embodiment. In this example, a person works out on a machine 1000 with a monitor 1002. The person can either take a picture when they start 1010 and stop 1012 and the system, method, and/or device will determine the amount of calories utilized or the person can take a picture of the monitor when it should calories used 1004 via the camera 150. This calculation can also be directly received from the device (e.g., step counter, elliptical machine, smart watch, phone, etc.). In addition, this calculation can be based on body signals of the person (e.g., heart rate, oxygen level, oxygen saturation, and/or any other work level measurement). In one example, the medical device system may comprise a medical device, sensor(s), and lead(s) coupling the sensor(s) to the medical device. In one embodiment, one or more sensors may be configured to collect one or more body signals (e.g., kinetic activity, blood oxygen levels, accelerometer, heart rate, blood pressure, etc.) from a patient relating to a work level (activity level) of the patient. Work level refers to a patient's energy consumption for any action/behavior performed by the patient, which may be measured from body movement, oxygen consumption, brain activity, body signals, and/or via camera 150. The activity level signal may be a neurologic signal (e.g., a kinetic signal or a brain activity signal), a metabolic signal an endocrine signal, an autonomic signal, and/or a tissue stress signal. Further, the system, device, and/or method can use a user profile and/or learned information via one or more engines to complete this calculation.

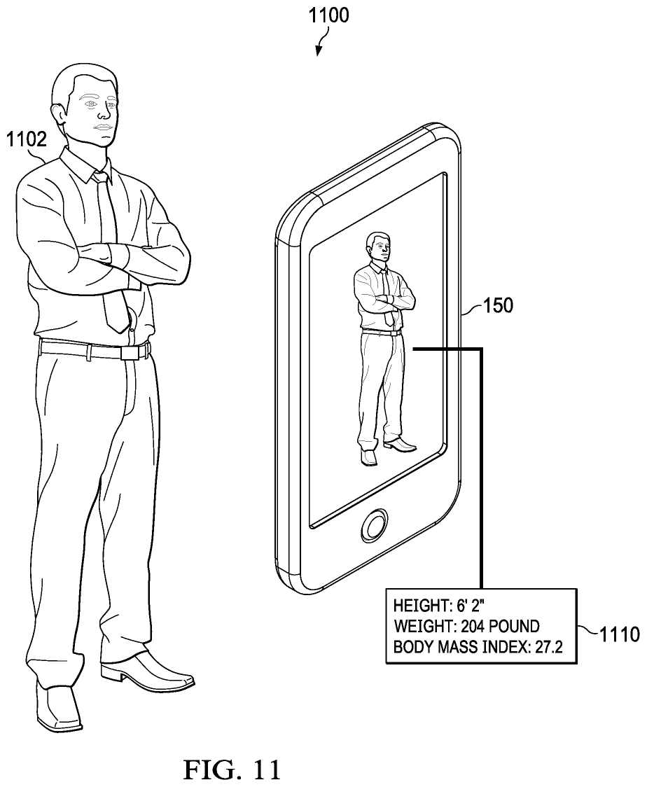

FIG. 11 is an illustration of a weight program process, according to one embodiment. In this example, a person can take a picture of themselves and the system, method, and/or device will generate a weight of the person, a body fat range for the person, a body mass index number, and a recommended daily calorie intake and exercise plan. In another example, a person can weight themselves and enter this data (e.g., manually and/or voice commands). In addition, the system, method, and/or device can communicate with a scale to have this information transmitted to it. Further, the system, method, and/or device can modify the recommended daily calorie intake and exercise plan based on a targeted weight loss and/or weight gain objective. In another example, the system, method, and/or device may project what the individual will look like after 2 weeks on the plan (e.g., projecting how the user's body will change), what the individual will look like after 3 weeks on the plan, what the individual will like after 4 weeks on the plan, what the individual will look like after Nth weeks on the plan, and/or what the individual will look like with an exercise plan also included. Further, a record (e.g., images, data, etc.) can be kept to show what the individual looked like in the being and all the progressive steps and what the person looked like at each step. In another example, the system, method, and/or device will take into account the medicine(s) and/or medical conditions (e.g., diabetes, cancer, etc.) a person is taking or has to develop this plan and/or generate warnings. Further, one or more body signals from the person can also be used to develop and/or modify this plan and/or generate warnings. For example, a person taking a first medicine (e.g., axitinib) has to monitor their blood pressure and cannot have grapefruit. In one example, a person on the first medicine takes a picture of a grapefruit and the system, method, and/or device generates a warning that this is grapefruit which has a problematic interaction with the first medicine—DO NOT EAT. In another example, a person on the first medicine takes a picture of French fries and the system, method, and/or device generates a warning that there is a lot of salt which may increase your blood pressure—USE CAUTION. In this example, while eating the French fries the system, method, and/or device receives one or more body signals that indicates that the person's blood pressure and/or heart rate is increasing. The system, method, and/or device generates a warning relating to same—Your Body Pressure is X—Please stop eating the salting food. In another example, a person is exercising and the system, method, and/or device receives one or more body signals that indicate that the person should reduce and/or stop exercising, the system, method, and/or device generates a warning relating to same—Your heart rate is X, Please slow and/or stop exercising. In another example, the system, method, and/or device may generate a warning based on a time of day a medicine is being taken (e.g., too close to the last dosage taken), taking a first medicine with a second medicine, not taking a medicine, exceeding the recommended daily dosage amount, one or more body signals indicating that it is unsafe to take the medicine, a food previously consumed (a meal was eaten in the last ½ hour), any combination thereof, and/or any other reason disclosed in this disclosure. In another example, the system, method, and/or device may make outfit recommendations based on the person body type.

In another example, the system, method, and/or device may track a history of consumed foods and any consequences that occur. For example, the last time you ate crabs you had stomach issues 4 hours later and/or needed to consume stomach medicine. In another example, 70 percent of the time you eat crabs, you have stomach issues and/or you take stomach medicine shortly thereafter. In another example, there is a historical pattern that if you eat pizza (e.g., three slices) and then have desert, you have diarrhea 30 percent of the time within 24 hours. Conversely, when you eat 1 slice of pizza and have desert, you have diarrhea 0 percent of the time. Whereas, when you eat 2 slices of pizza and have dessert, you have diarrhea 10 percent of the time within 24 hours. In another example, the system, method, and/or device may monitor the amount of any substance consumed. For example, the system, method, and/or device may determine and/or monitor that you have consumed a sugar amount above a threshold level. The system, method, and/or device may generate a warning that you are above a sugar amount for the day. In other examples, there could be a red meat threshold amount, a dairy threshold amount, a carbohydrate amount, a vegetable amount, a medicine amount (e.g., stomach medicine—exceed recommended dosage level, etc.), an exercise amount (e.g., 12,000 steps which normally causes knee pain), any amount of a first substance (e.g., nuts, grapefruit, shellfish, etc.) which the user is not permitted to consume, fried food, fast food, and/or any other consumable discussed in this disclosure, and/or any combination thereof may be utilized. In various other examples, the system, method, and/or device may monitor, track, generate warnings, and/or generate recommendations, and/or any other action items disclosed in this disclosure, and/or any combination thereof based on a user's health, a user's age, a user's profile, and/or any other data in this disclosure, and/or any combination thereof. In addition, a time of day may be utilized—don't eat anything after a first time (e.g., 7 PM—Causes sleeping problems, weight gain, gremlin problems, etc.).

In another example, the system, method, and/or device may generate one or more recommendations. For example, the system, method, and/or device may analyze the food on the plate and issue a recommendation that if you don't eat the fries that saves you 400 calories and 20 grams of fat. In another example, if you eat half the fries that saves you 200 calories and 10 grams of fat. In another example, if you don't eat the bread on your sandwich that saves you 300 calories and 20 grams of carbohydrates. In another example, the system, method, and/or device breaks down the calorie count for you—the burger is 600 calories, the fries are 400 calories, vegetables are 150 calories, the butter is 150 calories, the ranch dressing for the fries is 200 calories, and the sugar drink is 500 calories. This allows the user to know where the calories are before starting and/or while eating and/or after eating the food. In another example, the system, method, and/or device may make one or more recommendations based on the user's actions during the day. For example, based on your completed exercise, food already consumed, medicine consumed, and/or body signals, the system, method, and/or device recommends that you eat lightly butter fish with carrots and one glass of wine—ENJOY!!! In another example, the system, device, and/or method may associate specific foods with other actions. Stephen is eating a cheese steak so we should recommend an Eagles football game highlight reel. In another example, Stephen is drinking at Moe, therefore, we should communicate (e.g., phone, email, text, social media, etc.) with Larry and Curly that Stephen is at Moe's Bar—on contact Homer.

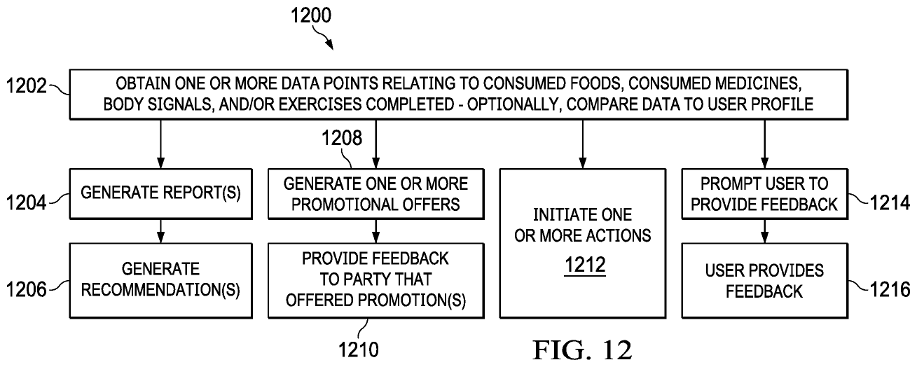

In FIG. 12, a calorie intake, medical intake, body signals monitoring, and/or exercise completed flow chart is shown. A method 1200 may include obtaining one or more data points relating to consumed foods, consumed medicines, one or more body signals, and/or exercise(s) completed (step 1202). Optionally, this data can be compared to the user profile. Further, this data may be obtained via camera 150, one or more body sensors, one or more smart devices (e.g., smart watch, smart phone, etc.), via user profile, via user data entry, and/or any combination thereof. The method 1200 may include generating one or more reports (step 1204). The method 1200 may include generating one or more recommendations (step 1206). The method 1200 may include generating one or more promotional offers (step 1208). The method 1200 may include providing feedback to one or more parties that offered the one or more promotional offers (step 1210). The method 1200 may include initiating one or more actions (step 1212). The method may include prompting the user to provide feedback (step 1214). The method 1200 may include the user providing feedback (step 1216). In one example, one or more user data points may be transmitted to a third party where the third party may develop one or more promotional items and/or customized offers for the use based on the user data points. The data points can be a user's diet, a user's preferred meals, a user's exercise routine, one or more restaurants a user has eaten at, a supermarket that the user utilizes, one or more medicines used, any data in this disclosure, and/or any combination thereof. In another example, the system, method, and/or device may be tied into a database (e.g., a calendar, a user profile, a shopping list, etc.) and used with a recipe for a first meal that is proposed to be made tonight and tied into the shopping list and/or a food inventory at home. Based on one or more of these data points, the system, method, and/or device may generate a warning that a first item (e.g., tomatoes, eggs, butter, etc.) is required for this proposed meal but that this first item is not on the shopping list nor in food inventory at home. In another example, the system, method, and/or device may help manage the food inventory. For example, the system, device, and/or method may issue a message that the eggs are 10 days old and about to expire. Therefore, these eggs should be eaten soon and new eggs should be put on the shopping list. In various examples, the system, method, and/or device may automatically add items to the shopping list, generate recipe suggestions to use the expiring food, etc.

In another example, the system, method, and/or device may obtain one or more body signals and/or samples to generate one or more reports, recommendations, displays, and/or warnings. For example, a blood pressure signal may be utilized to generate a warning related to salt and/or red meat intake for the user. In another example, a pee sample and/or defecate sample may be utilized to analyze the user's diet and generate one or more recommendations (e.g., eat less red meat, eat more fiber, drink less sugary drinks, etc.).

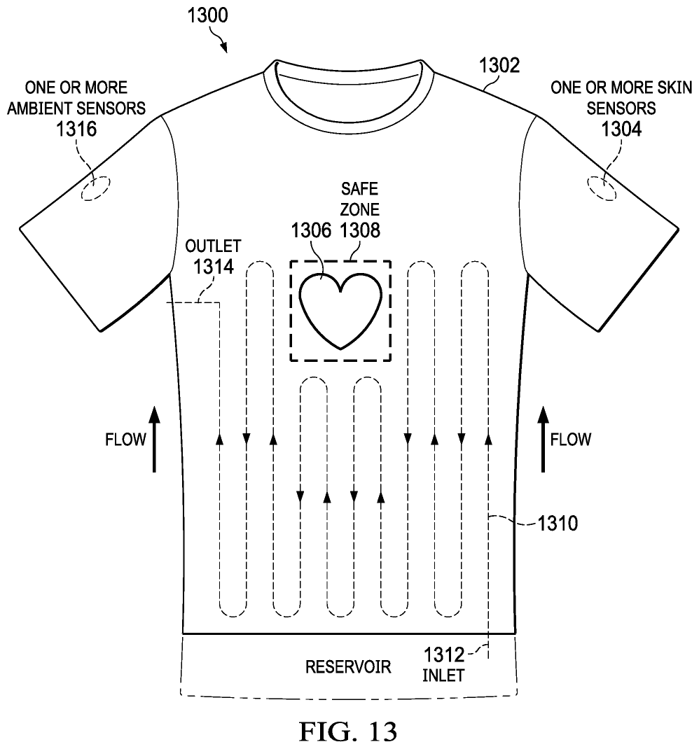

In one example, body temperature, heart rate, eye dilation, and/or medical monitoring equipment information may be utilized as part of the system's machine learning procedure. In various examples, the system, method, and/or device may obtain information from a camera, a smart device, the shirt shown in FIGS. 13-19, a user profile, user input, one or more sensors, derived data, calculated data, model data, crowdsourced data, machine learned data, machine learning engine, a third party source, any source described in this disclosure, and/or any combination thereof.

In one embodiment, a system, a method, and/or a device may include: one or more processors which receive at least a first image and a second image, the one or more processors may determine a starting calories content of a first consumable item from extracting data from the first image and an ending calories content of the first consumable item from extracting data from the second image, the one or more processors may determine a first consumed calories by calculating a difference between the starting calories content of the first consumable item and the ending calories content of the first consumable item.

In addition, the one or more processors may utilize at least one of: one or more engines; one or more lookup tables; one or more libraries; an Internet; a calorie counter program; user default settings; system default settings; a bar code; one or more scanned words; crowd sourced data; one or more machine learning systems; one or more voice commands, and one or more content identifiers to determine the starting calories content of the first consumable item and the ending calories content of the first consumable item. Further, the one or more processors may determine at least one of: a protein content; a sugar content; a vegetable content; a carbohydrate content; a vitamin content; and a nutritional value of at least one of the starting calories content of the first consumable item; the ending calories content of the first consumable item; and the first consumed calories. In addition, the system, the method, and/or the device may include a display and the one or more processors may compare the first consumed calories to a calorie target and generate a first report on the display relating to the comparison of the first consumed calories to the calorie target. In addition, the one or more processors may generate at least one of: one or more warnings; one or more recommendations; one or more promotional offers; one or more messages; and commands to initiate one or more actions based on the comparison of the first consumed calories to the calorie target. In addition, the one or more processors may receive at least a third image and a fourth image, the one or more processors are configured to determine a starting calories content of a second consumable item from extracting data from the third image and an ending calories content of the second consumable item from extracting data from the fourth image, the one or more processors may determine a second consumed calories by calculating a difference between the starting calories content of the second consumable item and the ending calories content of the second consumable item. In addition, the one or more processors may compare the second consumed calories to a calorie target and generate a second report on the display relating to the comparison of the second consumed calories to the calorie target. In addition, the one or more processors may generate at least one of: one or more warnings; one or more recommendations; one or more promotional offers; one or more messages; and commands to initiate one or more actions based on the comparison of the second consumed calories to the calorie target. In addition, the one or more processors may receive at least an Nth−1 image and an Nth image, the one or more processors are configured to determine a starting calories content of an Nth consumable item from extracting data from the Nth−1 image and an ending calories content of the Nth consumable item from extracting data from the Nth image, the one or more processors may to determine an Nth consumed calories by calculating a difference between the starting calories content of the Nth consumable item and the ending calories content of the Nth consumable item. In addition, the one or more processors may compare the Nth consumed calories to a calorie target and generate an Nth report on the display relating to the comparison of the Nth consumed calories to the calorie target. In addition, the one or more processors may generate at least one of: one or more warnings; one or more recommendations; one or more promotional offers; one or more messages; and commands to initiate one or more actions based on the comparison of the Nth consumed calories to the calorie target. In addition, the one or more processors may receive at least a third image and a fourth image, the one or more processors are configured to determine a starting calories content of a second consumable item from extracting data from the third image and an ending calories content of the second consumable item from extracting data from the fourth image, the one or more processors may determine a second consumed calories by calculating a difference between the starting calories content of the second consumable item and the ending calories content of the second consumable item; the one or more processors may receive at least an Nth−1 image and an Nth image, the one or more processors may determine a starting calories content of an Nth consumable item from extracting data from the Nth−1 image and an ending calories content of the Nth consumable item from extracting data from the Nth image, the one or more processors may determine an Nth consumed calories by calculating a difference between the starting calories content of the Nth consumable item and the ending calories content of the Nth consumable item; the one or more processors may compare the second consumed calories to a calorie target and generate a second report on the display relating to the comparison of the second consumed calories to the calorie target; the one or more processors may compare a total of the first consumed calories, the second consumed calories, and the Nth consumed calories to a calorie target and generate a consumed calorie report on the display relating to the comparison of the first consumed calories, the second consumed calories, and the Nth consumed calories to the calorie target. In addition, the one or more processors may complete one or more of the following items: determine one or more consumed medicines; determine one or more completed exercises; receive one or more body signals; and generate one or more warnings; one or more recommendations; one or more promotional offers; one or more messages; and initiate one or more actions based on the comparison of the first consumed calories, the second consumed calories, and the Nth consumed calories to the calorie target. In addition, the one or more processors may complete one or more of the following items: determine one or more consumed medicines; determine one or more completed exercises; and receive one or more body signals; and the one or more processors may generate at least one of: one or more warnings; one or more recommendations; one or more promotional offers; one or more messages; and commands to initiate one or more actions based on at least one of the determined medicines consumed; determined exercises completed; and received one or more body signals.

In another embodiment, a system, a method, and/or a device may include: one or more processors which receive at least a first image, the one or more processors may determine a starting calories content of a first consumable item from extracting data from the first image, the one or more processors may determine a contents of the first consumable item, and the one or more processors may determine a consumed medicine or a user's health condition.

In addition, the one or more processors may generate at least one of: one or more warnings; one or more recommendations; one or more messages; and commands to initiate one or more actions based on at least one of: a comparison of the contents of the first consumable item to at least one of the consumed medicine and the user's health condition; and a comparison of the starting calories content of the first consumable item to the consumed medicine or the user's health condition. In addition, the one or more processors may receive one or more body signals, the one or more processors may generate at least one of: one or more warnings; one or more recommendations; one or more messages; and commands to initiate one or more actions based on the one or more body signals. In addition, the one or more body signals may indicate at least one of: an increase in blood pressure; a decrease in blood pressure; an increase in heart rate; a decrease in heart rate; an increase in blood sugar level; a decrease in blood sugar level; an increase in a bodily function; and a decrease in a bodily function.

In another embodiment, a system, a method, and/or a device may include: one or more processors configured to:

receive at least a first image and a second image, the one or more processors configured to determine a starting calories content of a first consumable item from extracting data from the first image and an ending calories content of the first consumable item from extracting data from the second image, the one or more processors configured to determine a first consumed calories by calculating a difference between the starting calories content of the first consumable item and the ending calories content of the first consumable item, the one or more processors configured to add the first consumed calories to a calorie count;

receive data relating to a first exercise completed with a first utilized calories, the one or more processors configured to substrate the first utilized calories from the calorie count;

receive at least a third image and a fourth image, the one or more processors are configured to determine a starting calories content of a second consumable item from extracting data from the third image and an ending calories content of the second consumable item from extracting data from the fourth image, the one or more processors are configured to determine a second consumed calories by calculating a difference between the starting calories content of the second consumable item and the ending calories content of the second consumable item, the one or more processors configured to add the second consumed calories to the calorie count;

receive data relating to an Nth exercise completed with an Nth utilized calories, the one or more processors configured to substrate the Nth utilized calories from the calorie count;

receive at least an Nth−1 image and an Nth image, the one or more processors are configured to determine a starting calories content of an Nth consumable item from extracting data from the Nth−1 image and an ending calories content of the Nth consumable item from extracting data from the Nth image, the one or more processors are configured to determine an Nth consumed calories by calculating a difference between the starting calories content of the Nth consumable item and the ending calories content of the Nth consumable item, the one or more processors configured to add the Nth consumed calories to the calorie count; and

generate one or more reports based on at least one of: the first consumed calories, the second consumed calories, the Nth consumed calories, the first utilized calories, the Nth utilized calories, and the calorie count.

In addition, the one or more processors may generate at least one of: one or more warnings; one or more recommendations; one or more messages; and commands to initiate one or more actions based on the first consumed calories, the second consumed calories, the Nth consumed calories, the first utilized calories, the Nth utilized calories, and the calorie count.

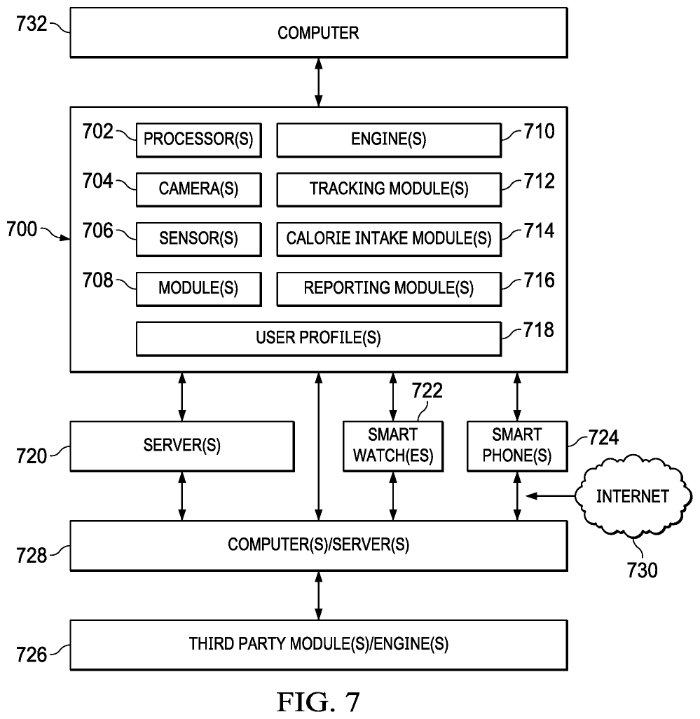

In another embodiment, a consumable (e.g., food, drinks, medicine, etc.) inventory management system, method, and/or device may be implemented by taking a picture of a receipt for one or more consumables. For example, a shopping receipt may be scanned and/or taken a picture of to allow the system, method, and/or device to recognize each individual consumable item. In another example, the shopping receipt may be scanned via a scanner. In another example, a bar code reader may be utilized when placing an item into inventory and/or removing an item from inventory. Further, the system, method, and/or device may utilize information from one or more images taken (See FIGS. 1-12) to remove items from the inventory. For example, a person has two eggs for breakfast, two pieces of toast, three pieces of bacon, and a glass of orange juice and takes a picture of this meal. Therefore, the inventory management system, method, and/or device would reduce the inventory by two eggs, two pieces of bread, three pieces of bacon, and the amount of orange juice in the glass. In another example, the inventory management system, method, and/or device may have items added and/or subtracted via one or more voice commands. For example, I am putting a new gallon of whole milk in the refrigerator and/or any language form. Therefore, a gallon of whole milk would be added to inventory list. In this example, the system, method, and/or device may have a default setting and/or capture the information from an image that the whole milk (and/or any other item—eggs, cheese, bread, canned food, meat, etc.) expires in 10 days. Therefore, the system, method, and/or device would send out a warning that one or more items is going to expire if not used when the date is approaching, remove the item from inventory after the expiration date, place the item on a shopping list after the expiration date, any other function in this disclosure, and/or any combination thereof.

In another example, the refrigerator and/or the pantry may have one or more sensors and/or one or more cameras that automatically track what is placed inside of the refrigerator and/or pantry. For example, the one or more cameras may identify that whole milk is being placed inside of the refrigerator. Further, the one or more sensors may identify that the whole milk is presently full based on weight and/or level of milk in the container. However, once the whole milk is utilized, the one or more cameras and/or one or more sensors can determine how much whole milk is left based on an image and/or the weight reading. In another example, two cans of tomatoes are placed inside the pantry, the one or more sensors and/or one or more cameras identifies that that there are two cans of tomatoes placed in the pantry with an expiration date of Mar. 1, 2020. Therefore, when this approaches a warning and/or message may be generated and/or a food recommendation option that uses the tomatoes may be generated. In addition, when one of these cans of tomatoes is removed from the pantry, the one or more sensors and/or one or more cameras identifies this and removes the can of tomatoes from the inventory list and/or adds a can of tomatoes to the shopping list.

In another example, the system, method, and/or device may generate food options based on the user's GPS location, the user's health, the user's calorie count, one or more body signals, based on the location of your friends, promotional offerings, a financial budget, a type of user (e.g., vegan), a time of day, the weather, the actual day (e.g., thanksgiving), a distance to restaurant, a wait time at a restaurant, restaurant hours, ratings of one or more restaurants, traffic to one or more restaurants, social buzz about one or more restaurants, social causes, friend's favorite places, someone you follow favorite places, diet mode user(s) are in—(no breads, etc.), one or more body signals, and/or other factor disclosed in this document, and/or any combination thereof.

For example, the system, method, and/or device may generate three consumable options based on the location of the user and the locations of the restaurants. In another example, the system, method, and/or device may generate three consumable options based on the location of the user and the locations of the restaurants and the user's calorie count for the day—the options could be a little meal (or healthier) option based on the calorie count being close to a target calorie count. In another example, the option could be a low sodium option based one or more body signals. In another example, the option could be a low sugar option based on one or more body is signals. In one example, based on the location of your three friends who are going to lunch with you the system, method, and/or device selections options based on locational convenience, one or more calorie counts for each individual, one or more health status of the individuals, and/or any combination thereof. In one example, an option may be recommended based on a user budget—meals under 15 dollars, etc. In another example, an option may be based on the weather—when it is sunny out the user likes to go to a restaurant on the beach. If it is not sunny out, then the user does not like doing that. In another example, an option may be based on a time of day—lighter meals after 6 PM, etc. In another example, an actual date may be a factor. For example, on a specific day (e.g., thanksgiving) the system, method, and/or device determines that the user will consume a large amount of calories at dinner, therefore, the system, method, and/or device may recommend consumable options for breakfast and lunch that are low calorie options.

In another example, the system, method, and/or device may generate a meal order command which sends a meal order to the restaurant based on user settings, voice commands, manual inputs, and/or any other characteristic disclosed in this document, and/or any combination thereof. In addition, this meal order command may be aligned with the time it will take to arrive at the restaurant. In other words, the meal will take 15 minutes to make but you are 30 minutes away. Therefore, instead of your food sitting for 15 if you ordered right now because you won't be there for another 30 minutes, the system, method, and/or device would delay your order for 15 minutes to align the meal completion time with your arrival. In this example, the system, method, and/or device may factor in traffic and/or the arrival of the first person and/or last person.

In one example, the system, method, and/or device may generate directions for one or more people (e.g., friends, family, business associates, etc.) based on the location of where the meal will be consumed. In another example, the system, method, and/or device may turn on an oven based on the user's GPS location, change the temperature of an item (e.g., crook pot) based on the user's GPS location, and/or start another item (e.g., coffee maker) based on the user's GPS location.

In another example, the system, method, and/or device may generate consumable options based on a user's schedule. For example, the system, method, and/or device may learn a person schedule based on a calendar and/or GPS data. In an example, the GPS data shows that every third week of the month the person goes to an offsite location (e.g., cabin) to spend the weekend. Therefore, the system, method, and/or device may learn the route to the cabin and recommend various consumable options based on this route, the user's GPS location, the user's health, the user's calorie count, one or more body signals, based on the location of your friends, promotional offerings, a financial budget, a type of user (e.g., vegan), a time of day, the weather, the actual day (e.g., thanksgiving), a distance to restaurant, a wait time at a restaurant, restaurant hours, ratings of one or more restaurants, traffic to one or more restaurants, social buzz about one or more restaurants, social causes, friend's favorite places, someone you follow favorite places, diet mode user(s) are in—(no breads, etc.), one or more body signals, and/or one or more devices at the cabin. For example, the cabin only has an X sized oven; therefore the options would be limited by this factor.

In another example, the user's calorie count and/or calorie count tracking charts may be shared with friends, a support group, a competition group, family, and/or any other group disclosed in this document, and/or any combination thereof. In another example, the system, method, and/or device may use and/or combine one or more user profiles and/or user default settings to generate one or more consumable option. For example, a newly formed couple may use this feature to select a consumable option that is acceptable to both parts of the couple.

In another example, the system, method, and/or device may generate directions to the nearest (and/or best and/or most appropriate) hospital based on one or more body signals.

In one situation, a person wearing may wear a normal shirt. In this example, the body is trying to maintain a normal body temperature (e.g., 98.6 F). In this example, the normal shirt help the body maintain the normal body temperature when the room (e.g., ambient temperature) is below the desired body temperature of 98.6 F. For example, if a person is in a room with a temperature of 72.0 F, then heat transferred will occur between the person and the room because the person is at a temperature of 98.6 F and the room is at a temperature of 72.0 F. Therefore, the person will transfer heat to the room. However, if this person is wearing the normal shirt, then heat transfer will be reduced because the shirt acts as an insulator to both the room and the person. Therefore, the person's body will expend less energy trying to keep the person's body temperature at 98.6 F.

In FIG. 13, an illustration of a shirt with a heat exchanging system is shown, according to one embodiment. In this example, a weight-loss shirt 1300 includes a shirt, a tube 1310 (and/or pipes, and/or any other heat exchanging material), a front-shirt inlet area 1302, and a front-shirt outlet area 1314. In this example, a safety zone 1308 is shown. The safety zone 1308 is any area where heat exchanging equipment should not be placed for health and/or comfort reasons and/or functions (e.g., potentially groin, private parts, head, etc.). In this example, the safety zone 1308 is around the heart. It should be noted that the safety zone 1308 may not be needed depending on the delta temperature utilized and/or the amount of energy used in the heat exchanger process. In this example, a first design configuration for the tube(s) 1310 is shown and that only the front of the shirt has tube(s) 1310. In one example, a reservoir area 1320 is also included. In another example, one or more ambient sensors 1316 and/or one or more skin sensors 1304 are utilized. The one or more ambient sensors 1316 are configured to measure one or more data points (e.g., temperature, humidity, air pressure, altitude, etc.) of the environment surrounding a person. Whereas, the one or more skin sensors 1304 are configured to measure one or more data points (e.g., temperature, moisture, skin stress, etc.) relating to the person's skin.

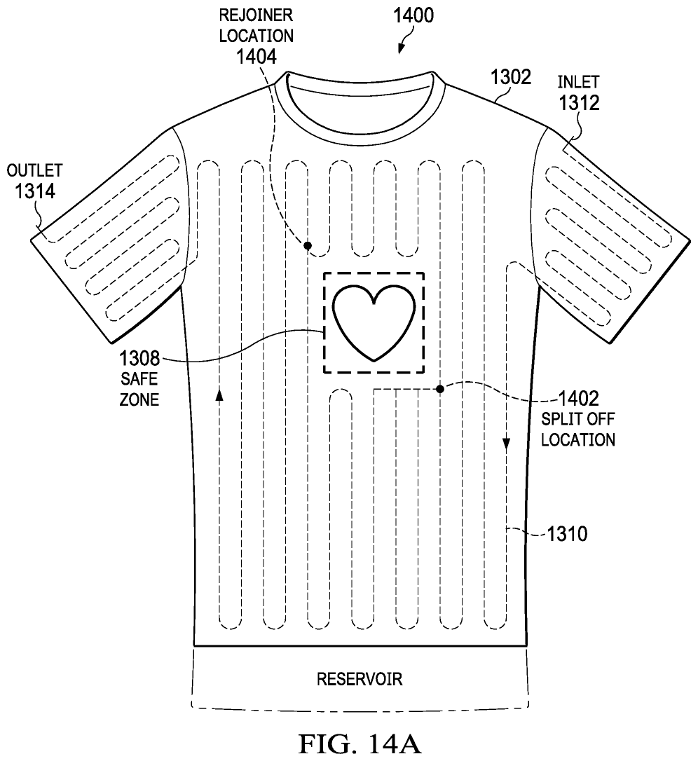

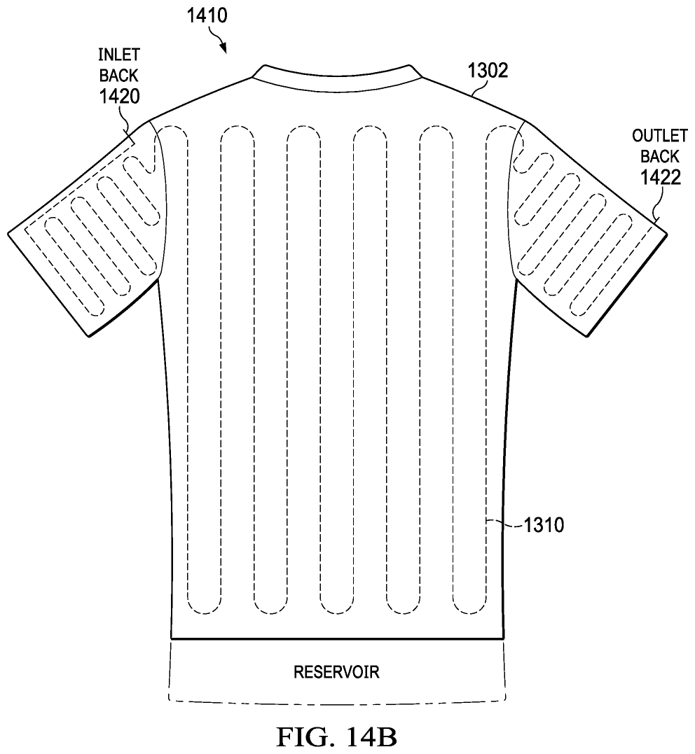

In FIG. 14A, an illustration of the front side of the disclosed shirt with a heat exchanging system is shown, according to one embodiment. In this example, the weight-loss shirt 1400 includes a shirt, the tube 1310 (and/or pipes, and/or any other heat exchanging material), the front-shirt inlet area 1302, and the front-shirt outlet area 1314. In this example, the safety zone 1308 is shown. Further, a split off location 1402 and a rejoiner location 1404 are shown. In this example, the split off location 1402 is where one or more tube(s) 1310 split into two or more lines. Further, the rejoiner location 1404 is where one or more lines rejoin one or more tube(s) 1302. In this example, a second design configuration for the tube(s) 1310 is shown and this example includes both a front configuration (FIG. 14A) and a back configuration (FIG. 14B) where the back configuration includes a back inlet area 1420 and a back outlet area 1422. It should be noted that the tube(s) 1310 can be in any configuration, angle, layering, spacing, number, thickness, tininess, size, volume, etc.

In another example, the weight-loss shirt 1400 includes a shirt, the tube 1310 (and/or pipes, and/or any other heat exchanging material), the front-shirt inlet area 1312, and the front-shirt outlet area 1314. In this example, there is no safety zone 1308 because the delta in temperature does not require one. In this example, a third design configuration for the tube(s) may be a horizontal configuration, a vertical and horizontal configuration, a snaked configuration, a looped configuration, an Z-shaped configuration, a circle configuration, an L-Shaped configuration, and/or any combination thereof. For brevity, each of these configurations is not shown in a figure but may be utilized as claim limitations.

In another example, a vest includes the tube(s), a reservoir, an inlet area, and an outlet to back area. In this example, the reservoir is filed with one or more heat exchanging elements (e.g., water, liquid heat exchanging element, gaseous heat exchanging element, and/or any combination thereof). In another example, the back side of the vest with an inlet area to the back of the shirt and an outlet area to a reservoir area may be utilized.

In another example, the shirt includes a balloon device which fills up with water (and/or any heat exchanging material) from the reservoir via an inlet. The water travels to the back of the shirt via an outlet area and fills up the second balloon device on the back of the shirt. In another example, the balloon device and the second balloon device are one device. The water and/or heat exchanging material are released to the reservoir via an outlet area. In one example, the tubes and the balloons may be combined and utilized at the same time. Further, the tubes may be flexible, rigid, and/or a combination thereof. For example, the flexible tube may be utilized in areas where kinks are not likely. Whereas, rigid tubes may be utilized in areas to reduce the risk of kinks. In addition, a ribbing material and/or design may be utilized to reduce the risk of kinks and to increase the pleasure/conform of the fit. Further, the heat exchange material could be conditioned air and/or air pushed through the tubes and/or the balloons. Further, the heat exchange material could be any combination thereof. For example, cooled air could be utilized via the tubes and/or balloons to lower a person's body temperature and/or skin and/or body temperature. Further, heated air could be utilized via the tubes and/or balloons to increase a person's body temperature and/or skin and/or body temperature. In addition, wires could be utilized to increase (e.g., heat) a person's body temperature and/or skin and/or body temperature. Further, a fan could be utilized to decrease (e.g., cool) a person's body temperature and/or skin and/or body temperature and/or exchangeable ice packs that air blows over. The wires and fan configurations may be combined, along with any other element in this disclosure.

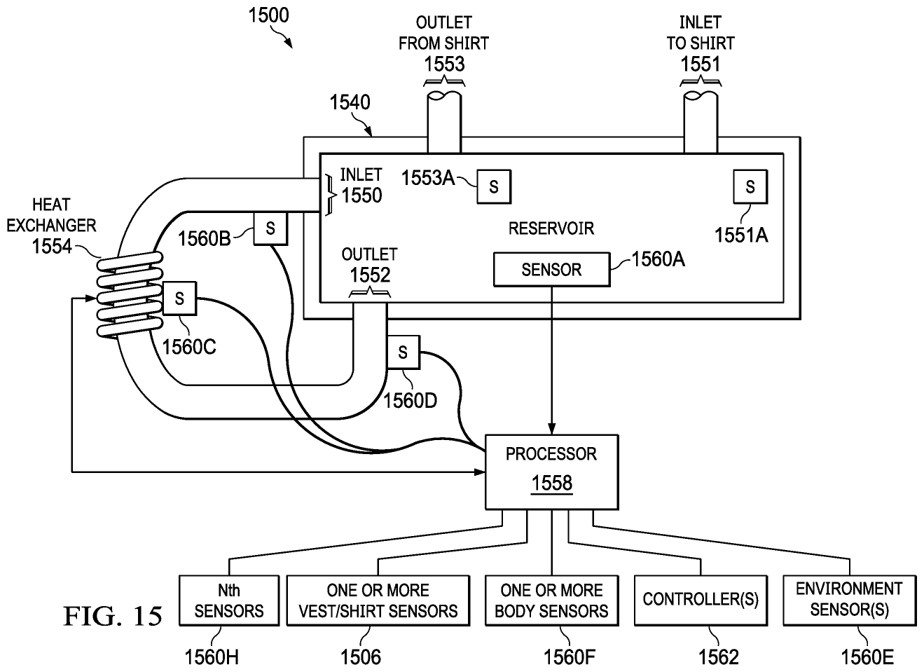

In FIG. 15, an illustration of a heat exchanging system, one or more processors 1558, one or more sensors (e.g., Nth sensors 1560H, one or more vest/shirt/hat/pants/gloves/toupee sensors 1506, one or more body sensors 1560F, and/or one or more environment sensors 1560E), and a controller 1562 is shown, according to one embodiment. In this example, the reservoir 1540 has an outlet 1552 and an inlet area 1550. The outlet 1552 draws the heat exchanging material to the heat exchanger 1554 which makes the heat exchanging material either cooler or warmer depending on one of the temperature of the heat exchanging material in the reservoir 1540, data from one or more sensors 1506, the mode the system is presently utilizing, a flow rate in the tube(s) 1310, the flow rate into and/or out of the reservoir 1540, a skin and/or body temperature of the wearer, a room temperature, a delta temperature between the skin and/or body temperature and the normal body temperature, a delta temperature between the skin and/or body temperature and the ambient temperature, a delta temperature between the ambient temperature and the normal body temperature, and/or any combination thereof. In this example, the reservoir 1540 may include a reservoir sensor 1560A, an outlet sensor 1560D, a heat exchanger sensor 1560C, an inlet sensor 1560B, a shirt inlet 1551, a shirt inlet sensor 1551A, a shirt outlet 1553, a shirt outlet sensor 1553A, and/or any other sensor, processor, inlet area, outlet area, or controller.

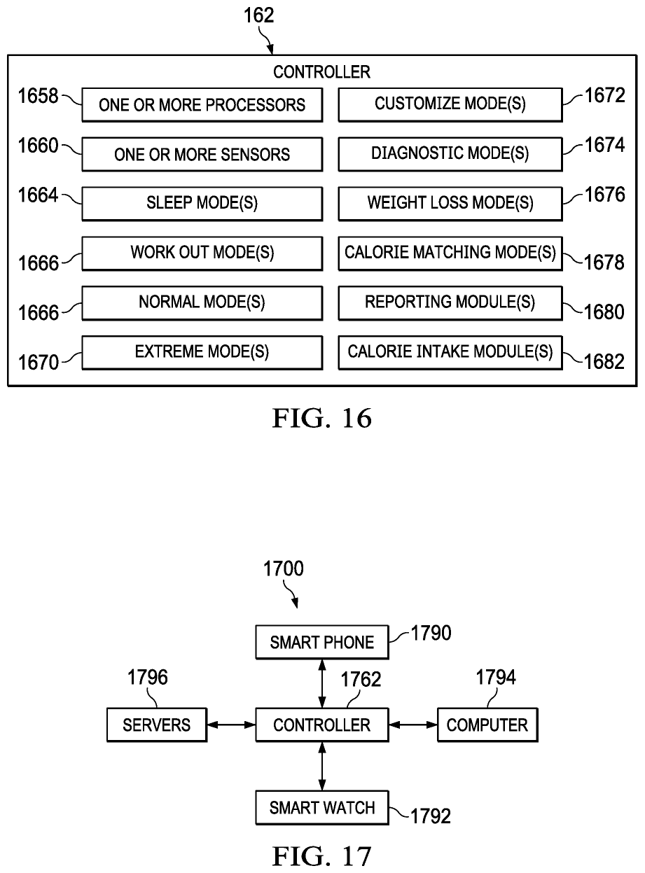

In FIG. 16, an illustration of a controller is shown, according to one embodiment. In this example, the controller 1662 includes one or more processors 1658, one or more sensors 1660, a sleep mode 1664, a work out mode 1666, a normal mode 1668, an extreme mode 1670, a customize mode 1672, a diagnostic mode 1674, a weight loss mode 1676, a calorie matching mode 1678, a reporting module 1680, a calorie intake module 1682, any other mode, any other module, and/or any combination thereof.

In another example, the shirt and/or vest in connected to an external heat exchanging via one or more connection devices (e.g., tubes, etc.). In this example, since the heat exchanger can be any size, additional calories can be burned by the person because of the additional heat loss and/or heat gain generated by the heat exchanger.

In another example, a pant includes an inlet area, tube(s), and an outlet to back area. Further, the pant can be combined with the reservoir. In addition, the back side of the pant may be utilized with an inlet from front area and an outlet of front area.

In FIG. 17, illustrations of the controller interacting with various systems are shown, according to various embodiments. In various examples, the controller 1762 may interact with a smart phone 1790, a smart watch 1792, a computer 1794, one or more servers 1796, one or more body data sensors, one or more user profiles, one or more third parties, one or more exercise devices, one or more skin sensors, one or more environmental sensors, one or more vest/shirt/hat/pants/gloves/toupee, one or more vest/shirt/hat/pants/gloves/toupee sensors, any other device and/or sensors described in this disclosure, and/or any combination thereof. For example, one or more of these devices may display calories utilized, calories intake, daily calorie target, daily calorie total, one or more graphs of data, reminders relating to goals, calorie intake, calories utilized, motivational messages, medicine data, body signals data, exercise data, and/or any other data in this disclosure.

In one example, the shirt (and/or vest, and/or pant and/or etc.) may be easily connected and/or discounted from the heat exchanging system via one or more connecting devices. In another example, this device can be utilized with a bra or other undergarment to keep an area cool.

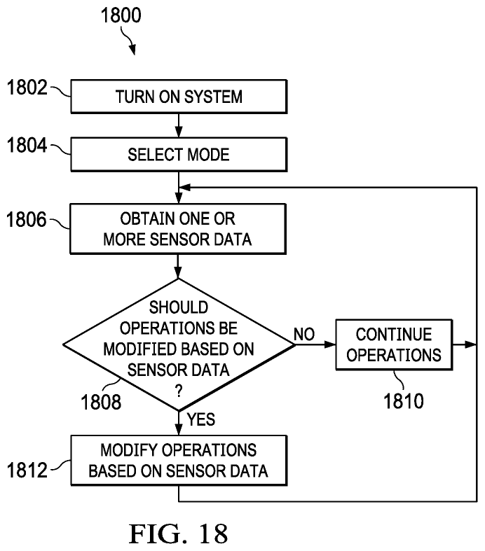

In FIG. 18, a process flow is shown, according to one embodiment. A method 1800 may include turning the system on (step 1802). The method 1800 may include selecting a mode of operation (step 1804). The method 1800 may include obtaining one or more sensor data (step 1806). The method 1800 may include determining via one or more processors whether an operation should be modified based on one or more sensor data and/or input data (step 1808). If the operation should not be modified based on one or more sensor data and/or input data, then the method may continue operations (step 1810) and return to step 1806. If the operation should be modified based on one or more sensor data and/or input data, then the method may modify the operations based on the sensor data and/or input data (step 1812) and return to step 1806. For example, a person may select a sleep mode. In the sleep mode, sensors may determine at what stage of sleep the person is in. Further, in sleep mode, the system, method, and/or device may not operate (e.g., implement a cooling and/or heating procedure) until the person is asleep and/or in a specific sleeping stage. Further, the sensors may provide data which indicates that the person is waking up, uncomfortable, and/or any other body condition and modify the cooling and/or heating procedures to maintain a sleeping stage for the person. In another example, in the sleep mode, the system, method, and/or device may operate (e.g., implement a cooling and/or heating procedure) when the person is still awake but the cooling and/or heating procedures may utilize a ramp up function for implementation, may utilize a step function for implementation, may utilized a customized function for implementation (e.g., step up to level X and then ramp to level Y, etc.), may utilized an oscillating function for implementation, may utilized a delay function that does not reach the sleeping stage before implementation, may utilized a delay function that reaches the sleeping stage with a buffer factor of time (e.g., 1 minute, 2, minutes, 5 minutes, 10 minutes, 1 hour, etc.) before implementation of the cooling and/or heating procedures, and/or any combination thereof. In another example, the system, method, and/or device may communicate with a smart phone, smart watch, external device, and/or a computer to determine the stage of sleep a person is in. In this example, the smart phone, smart watch, external device, and/or a computer may utilize sound, heart beat, breathing, and/or any other sensed data to determine the sleep stage. In various examples, the system, device (e.g., shirt), and/or method makes the body work harder than the body would normally work. In other words, if the body normally receives help from the shirt to stay warm and/or cool down, the device should do that exact opposite and make the body work a little harder. In another example, the system, method, and/or device may oscillate from decreasing the body temperature by a specific amount (e.g., 2 degrees) to increasing the body temperature by a specific amount (e.g., 2 degrees). Any of the temperature disclosed in this document and/or temperature ranges may be utilized in an oscillating embodiment, a first non-oscillating embodiment (e.g., decrease body temperature), and/or a second non-oscillating embodiment (e.g., increase body temperature).

In another example, the system, method, and/or device may implement the cooling and/or heating procedures and receive data relating to the ambient temperature and change the process. For example, the system, method, and/or device may be implementing a cooling procedure that targets a 2 degree drop in skin and/or body temperature because the ambient temperature is 72 degrees in the room. In this example, the body needs to work harder because the ambient temperature is less than the normal body temperature of 98.6 F. Therefore, adding load to the body by targeting a 2 degree drop in skin and/or body temperature will increase the amount of work the body is required to do which increases the amount of calories the body burns to complete this work. However, the person goes outside where the ambient temperature is 100 degrees, therefore, targeting a 2 degree drop in skin and/or body temperature would lower the amount of the work the body needs to do which would reduce the amount of work the body needs to complete—thereby reducing the amount of calories burned by the body. Therefore, the system, method, and/or device would modify the parameter of operations. In one example, the system, method, and/or device would shut off. In another example, the system, method, and/or device may target a 1 degree increase in skin and/or body temperature (e.g., the procedure would go to a heating procedure from a cooling procedure) which requires the body to utilized more calories to cool down the body because the outside ambient temperature is 100 degrees.

In another example, a work-out mode could be utilized which would increase the person's body temperature and body skin and/or body temperature during a workout which increases the amount of calories utilized during a workout based on the body needing to expended additional calories to reduce the person's body temperature and body skin and/or body temperature—these additional calories are in addition to any calories needed to complete the workout.

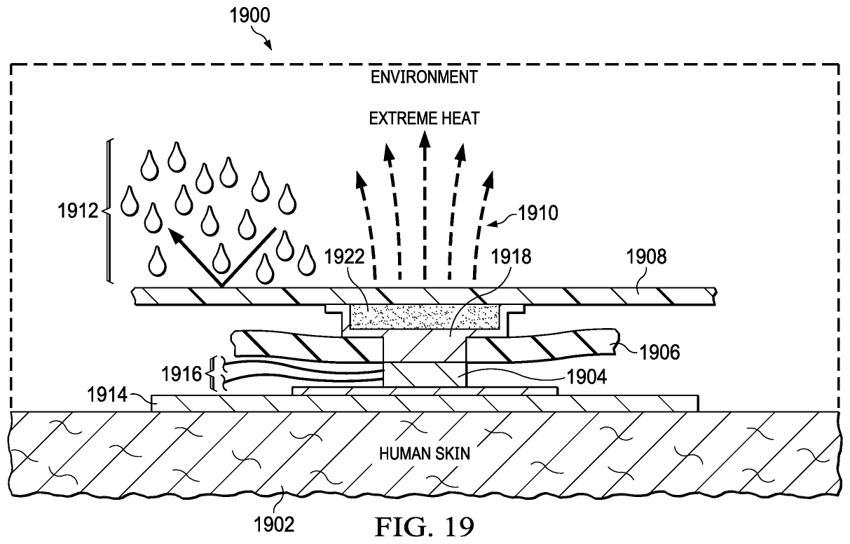

FIG. 19 is an illustration of Peltier based method, system, and/or device, according to various embodiments. In this example, the system, method, and/or device use a Peltier unit 1900. The Peltier unit 1900 includes an outer layer 1908 which may be water proof and/or breathable, an insulation layer 1906, a thermoelectric layer 1904, a heat transferring/absorbing material 1922, a heat sink 1918, a soft electrical wires 1916, and a thermally conductive material 1914. The Peltier unit 1900 is placed in proximate to the skin 1902. In this example, the water proofing is shown by reference number 1912 and the heat is shown by reference number 1910. Thermoelectric cooling uses the Peltier effect to create a heat flux at the junction of two different types of materials. A Peltier cooler, heater, or thermoelectric heat pump is a solid-state active heat pump which transfers heat from one side of the device to the other, with consumption of electrical energy, depending on the direction of the current.

It should be noted that any target change in skin and/or body temperature can be utilized. Various examples include +/−0.100, degrees, +/−0.101 degrees, +/−0.102 degrees, +/−0.103 degrees, +/−0.104 degrees, +/−0.105 degrees, +/−0.106 degrees, +/−0.107 degrees, +/−0.108 degrees, +/−0.109 degrees, +/−0.110 degrees, +/−0.111 degrees, +/−0.112 degrees, +/−0.113 degrees, +/−0.114 degrees, +/−0.115 degrees, +/−0.116 degrees, +/−0.117 degrees, +/−0.118 degrees, +/−0.119 degrees, +/−0.120 degrees, +/−0.121 degrees, +/−0.122 degrees, +/−0.123 degrees, +/−0.124 degrees, +/−0.125 degrees, +/−0.126 degrees, +/−0.127 degrees, +/−0.128 degrees, +/−0.129 degrees, +/−0.130 degrees, +/−0.131 degrees, +/−0.132 degrees, +/−0.133 degrees, +/−0.134 degrees, +/−0.135 degrees, +/−0.136 degrees, +/−0.137 degrees, +/−0.138 degrees, +/−0.139 degrees, +/−0.140 degrees, +/−0.141 degrees, +/−0.142 degrees, +/−0.143 degrees, +/−0.144 degrees, +/−0.145 degrees, +/−0.146 degrees, +/−0.147 degrees, +/−0.148 degrees, +/−0.149 degrees, +/−0.150 degrees, +/−0.151 degrees, +/−0.152 degrees, +/−0.153 degrees, +/−0.154 degrees, +/−0.155 degrees, +/−0.156 degrees, +/−0.157 degrees, +/−0.158 degrees, +/−0.159 degrees, +/−0.160 degrees, +/−0.161 degrees, +/−0.162 degrees, +/−0.163 degrees, +/−0.164 degrees, +/−0.165 degrees, +/−0.166 degrees, +/−0.167 degrees, +/−0.168 degrees, +/−0.169 degrees, +/−0.170 degrees, +/−0.171 degrees, +/−0.172 degrees, +/−0.173 degrees, +/−0.174 degrees, +/−0.175 degrees, +/−0.176 degrees, +/−0.177 degrees, +/−0.178 degrees, +/−0.179 degrees, +/−0.180 degrees, +/−0.181 degrees, +/−0.182 degrees, +/−0.183 degrees, +/−0.184 degrees, +/−0.185 degrees, +/−0.186 degrees, +/−0.187 degrees, +/−0.188 degrees, +/−0.189 degrees, +/−0.190 degrees, +/−0.191 degrees, +/−0.192 degrees, +/−0.193 degrees, +/−0.194 degrees, +/−0.195 degrees, +/−0.196 degrees, +/−0.197 degrees, +/−0.198 degrees, +/−0.199 degrees, +/−0.200 degrees, degrees, +/−0.201 degrees, +/−0.202 degrees, +/−0.203 degrees, +/−0.204 degrees, +/−0.205 degrees, +/−0.206 degrees, +/−0.207 degrees, +/−0.208 degrees, +/−0.209 degrees, +/−0.210 degrees, +/−0.211 degrees, +/−0.212 degrees, +/−0.213 degrees, +/−0.214 degrees, +/−0.215 degrees, +/−0.216 degrees, +/−0.217 degrees, +/−0.218 degrees, +/−0.219 degrees, +/−0.220 degrees, +/−0.221 degrees, +/−0.222 degrees, +/−0.223 degrees, +/−0.224 degrees, +/−0.225 degrees, +/−0.226 degrees, +/−0.227 degrees, +/−0.228 degrees, +/−0.229 degrees, +/−0.230 degrees, +/−0.231 degrees, +/−0.232 degrees, +/−0.233 degrees, +/−0.234 degrees, +/−0.235 degrees, +/−0.236 degrees, +/−0.237 degrees, +/−0.238 degrees, +/−0.239 degrees, +/−0.240 degrees, +/−0.241 degrees, +/−0.242 degrees, +/−0.243 degrees, +/−0.244 degrees, +/−0.245 degrees, +/−0.246 degrees, +/−0.247 degrees, +/−0.248 degrees, +/−0.249 degrees, +/−0.250 degrees, +/−0.251 degrees, +/−0.252 degrees, +/−0.253 degrees, +/−0.254 degrees, +/−0.255 degrees, +/−0.256 degrees, +/−0.257 degrees, +/−0.258 degrees, +/−0.259 degrees, +/−0.260 degrees, +/−0.261 degrees, +/−0.262 degrees, +/−0.263 degrees, +/−0.264 degrees, +/−0.265 degrees, +/−0.266 degrees, +/−0.267 degrees, +/−0.268 degrees, +/−0.269 degrees, +/−0.270 degrees, +/−0.271 degrees, +/−0.272 degrees, +/−0.273 degrees, +/−0.274 degrees, +/−0.275 degrees, +/−0.276 degrees, +/−0.277 degrees, +/−0.278 degrees, +/−0.279 degrees, +/−0.280 degrees, +/−0.281 degrees, +/−0.282 degrees, +/−0.283 degrees, +/−0.284 degrees, +/−0.285 degrees, +/−0.286 degrees, +/−0.287 degrees, +/−0.288 degrees, +/−0.289 degrees, +/−0.290 degrees, +/−0.291 degrees, +/−0.292 degrees, +/−0.293 degrees, +/−0.294 degrees, +/−0.295 degrees, +/−0.296 degrees, +/−0.297 degrees, +/−0.298 degrees, +/−0.299 degrees, +/−0.300 degrees, degrees, +/−0.301 degrees, +/−0.302 degrees, +/−0.303 degrees, +/−0.304 degrees, +/−0.305 degrees, +/−0.306 degrees, +/−0.307 degrees, +/−0.308 degrees, +/−0.309 degrees, +/−0.310 degrees, +/−0.311 degrees, +/−0.312 degrees, +/−0.313 degrees, +/−0.314 degrees, +/−0.315 degrees, +/−0.316 degrees, +/−0.317 degrees, +/−0.318 degrees, +/−0.319 degrees, +/−0.320 degrees, +/−0.321 degrees, +/−0.322 degrees, +/−0.323 degrees, +/−0.324 degrees, +/−0.325 degrees, +/−0.326 degrees, +/−0.327 degrees, +/−0.328 degrees, +/−0.329 degrees, +/−0.330 degrees, +/−0.331 degrees, +/−0.332 degrees, +/−0.333 degrees, +/−0.334 degrees, +/−0.335 degrees, +/−0.336 degrees, +/−0.337 degrees, +/−0.338 degrees, +/−0.339 degrees, +/−0.340 degrees, +/−0.341 degrees, +/−0.342 degrees, +/−0.343 degrees, +/−0.344 degrees, +/−0.345 degrees, +/−0.346 degrees, +/−0.347 degrees, +/−0.348 degrees, +/−0.349 degrees, +/−0.350 degrees, +/−0.351 degrees, +/−0.352 degrees, +/−0.353 degrees, +/−0.354 degrees, +/−0.355 degrees, +/−0.356 degrees, +/−0.357 degrees, +/−0.358 degrees, +/−0.359 degrees, +/−0.360 degrees, +/−0.361 degrees, +/−0.362 degrees, +/−0.363 degrees, +/−0.364 degrees, +/−0.365 degrees, +/−0.366 degrees, +/−0.367 degrees, +/−0.368 degrees, +/−0.369 degrees, +/−0.370 degrees, +/−0.371 degrees, +/−0.372 degrees, +/−0.373 degrees, +/−0.374 degrees, +/−0.375 degrees, +/−0.376 degrees, +/−0.377 degrees, +/−0.378 degrees, +/−0.379 degrees, +/−0.380 degrees, +/−0.381 degrees, +/−0.382 degrees, +/−0.383 degrees, +/−0.384 degrees, +/−0.385 degrees, +/−0.386 degrees, +/−0.387 degrees, +/−0.388 degrees, +/−0.389 degrees, +/−0.390 degrees, +/−0.391 degrees, +/−0.392 degrees, +/−0.393 degrees, +/−0.394 degrees, +/−0.395 degrees, +/−0.396 degrees, +/−0.397 degrees, +/−0.398 degrees, +/−0.399 degrees, +/−0.400 degrees, degrees, +/−0.401 degrees, +/−0.402 degrees, +/−0.403 degrees, +/−0.404 degrees, +/−0.405 degrees, +/−0.406 degrees, +/−0.407 degrees, +/−0.408 degrees, +/−0.409 degrees, +/−0.410 degrees, +/−0.411 degrees, +/−0.412 degrees, +/−0.413 degrees, +/−0.414 degrees, +/−0.415 degrees, +/−0.416 degrees, +/−0.417 degrees, +/−0.418 degrees, +/−0.419 degrees, +/−0.420 degrees, +/−0.421 degrees, +/−0.422 degrees, +/−0.423 degrees, +/−0.424 degrees, +/−0.425 degrees, +/−0.426 degrees, +/−0.427 degrees, +/−0.428 degrees, +/−0.429 degrees, +/−0.430 degrees, +/−0.431 degrees, +/−0.432 degrees, +/−0.433 degrees, +/−0.434 degrees, +/−0.435 degrees, +/−0.436 degrees, +/−0.437 degrees, +/−0.438 degrees, +/−0.439 degrees, +/−0.440 degrees, +/−0.441 degrees, +/−0.442 degrees, +/−0.443 degrees, +/−0.444 degrees, +/−0.445 degrees, +/−0.446 degrees, +/−0.447 degrees, +/−0.448 degrees, +/−0.449 degrees, +/−0.450 degrees, +/−0.451 degrees, +/−0.452 degrees, +/−0.453 degrees, +/−0.454 degrees, +/−0.455 degrees, +/−0.456 degrees, +/−0.457 degrees, +/−0.458 degrees, +/−0.459 degrees, +/−0.460 degrees, +/−0.461 degrees, +/−0.462 degrees, +/−0.463 degrees, +/−0.464 degrees, +/−0.465 degrees, +/−0.466 degrees, +/−0.467 degrees, +/−0.468 degrees, +/−0.469 degrees, +/−0.470 degrees, +/−0.471 degrees, +/−0.472 degrees, +/−0.473 degrees, +/−0.474 degrees, +/−0.475 degrees, +/−0.476 degrees, +/−0.477 degrees, +/−0.478 degrees, +/−0.479 degrees, +/−0.480 degrees, +/−0.481 degrees, +/−0.482 degrees, +/−0.483 degrees, +/−0.484 degrees, +/−0.485 degrees, +/−0.486 degrees, +/−0.487 degrees, +/−0.488 degrees, +/−0.489 degrees, +/−0.490 degrees, +/−0.491 degrees, +/−0.492 degrees, +/−0.493 degrees, +/−0.494 degrees, +/−0.495 degrees, +/−0.496 degrees, +/−0.497 degrees, +/−0.498 degrees, +/−0.499 degrees, +/−0.500 degrees, degrees, +/−0.501 degrees, +/−0.502 degrees, +/−0.503 degrees, +/−0.504 degrees, +/−0.505 degrees, +/−0.506 degrees, +/−0.507 degrees, +/−0.508 degrees, +/−0.509 degrees, +/−0.510 degrees, +/−0.511 degrees, +/−0.512 degrees, +/−0.513 degrees, +/−0.514 degrees, +/−0.515 degrees, +/−0.516 degrees, +/−0.517 degrees, +/−0.518 degrees, +/−0.519 degrees, +/−0.520 degrees, +/−0.521 degrees, +/−0.522 degrees, +/−0.523 degrees, +/−0.524 degrees, +/−0.525 degrees, +/−0.526 degrees, +/−0.527 degrees, +/−0.528 degrees, +/−0.529 degrees, +/−0.530 degrees, +/−0.531 degrees, +/−0.532 degrees, +/−0.533 degrees, +/−0.534 degrees, +/−0.535 degrees, +/−0.536 degrees, +/−0.537 degrees, +/−0.538 degrees, +/−0.539 degrees, +/−0.540 degrees, +/−0.541 degrees, +/−0.542 degrees, +/−0.543 degrees, +/−0.544 degrees, +/−0.545 degrees, +/−0.546 degrees, +/−0.547 degrees, +/−0.548 degrees, +/−0.549 degrees, +/−0.550 degrees, +/−0.551 degrees, +/−0.552 degrees, +/−0.553 degrees, +/−0.554 degrees, +/−0.555 degrees, +/−0.556 degrees, +/−0.557 degrees, +/−0.558 degrees, +/−0.559 degrees, +/−0.560 degrees, +/−0.561 degrees, +/−0.562 degrees, +/−0.563 degrees, +/−0.564 degrees, +/−0.565 degrees, +/−0.566 degrees, +/−0.567 degrees, +/−0.568 degrees, +/−0.569 degrees, +/−0.570 degrees, +/−0.571 degrees, +/−0.572 degrees, +/−0.573 degrees, +/−0.574 degrees, +/−0.575 degrees, +/−0.576 degrees, +/−0.577 degrees, +/−0.578 degrees, +/−0.579 degrees, +/−0.580 degrees, +/−0.581 degrees, +/−0.582 degrees, +/−0.583 degrees, +/−0.584 degrees, +/−0.585 degrees, +/−0.586 degrees, +/−0.587 degrees, +/−0.588 degrees, +/−0.589 degrees, +/−0.590 degrees, +/−0.591 degrees, +/−0.592 degrees, +/−0.593 degrees, +/−0.594 degrees, +/−0.595 degrees, +/−0.596 degrees, +/−0.597 degrees, +/−0.598 degrees, +/−0.599 degrees, +/−0.600 degrees, degrees, +/−0.601 degrees, +/−0.602 degrees, +/−0.603 degrees, +/−0.604 degrees, +/−0.605 degrees, +/−0.606 degrees, +/−0.607 degrees, +/−0.608 degrees, +/−0.609 degrees, +/−0.610 degrees, +/−0.611 degrees, +/−0.612 degrees, +/−0.613 degrees, +/−0.614 degrees, +/−0.615 degrees, +/−0.616 degrees, +/−0.617 degrees, +/−0.618 degrees, +/−0.619 degrees, +/−0.620 degrees, +/−0.621 degrees, +/−0.622 degrees, +/−0.623 degrees, +/−0.624 degrees, +/−0.625 degrees, +/−0.626 degrees, +/−0.627 degrees, +/−0.628 degrees, +/−0.629 degrees, +/−0.630 degrees, +/−0.631 degrees, +/−0.632 degrees, +/−0.633 degrees, +/−0.634 degrees, +/−0.635 degrees, +/−0.636 degrees, +/−0.637 degrees, +/−0.638 degrees, +/−0.639 degrees, +/−0.640 degrees, +/−0.641 degrees, +/−0.642 degrees, +/−0.643 degrees, +/−0.644 degrees, +/−0.645 degrees, +/−0.646 degrees, +/−0.647 degrees, +/−0.648 degrees, +/−0.649 degrees, +/−0.650 degrees, +/−0.651 degrees, +/−0.652 degrees, +/−0.653 degrees, +/−0.654 degrees, +/−0.655 degrees, +/−0.656 degrees, +/−0.657 degrees, +/−0.658 degrees, +/−0.659 degrees, +/−0.660 degrees, +/−0.661 degrees, +/−0.662 degrees, +/−0.663 degrees, +/−0.664 degrees, +/−0.665 degrees, +/−0.666 degrees, +/−0.667 degrees, +/−0.668 degrees, +/−0.669 degrees, +/−0.670 degrees, +/−0.671 degrees, +/−0.672 degrees, +/−0.673 degrees, +/−0.674 degrees, +/−0.675 degrees, +/−0.676 degrees, +/−0.677 degrees, +/−0.678 degrees, +/−0.679 degrees, +/−0.680 degrees, +/−0.681 degrees, +/−0.682 degrees, +/−0.683 degrees, +/−0.684 degrees, +/−0.685 degrees, +/−0.686 degrees, +/−0.687 degrees, +/−0.688 degrees, +/−0.689 degrees, +/−0.690 degrees, +/−0.691 degrees, +/−0.692 degrees, +/−0.693 degrees, +/−0.694 degrees, +/−0.695 degrees, +/−0.696 degrees, +/−0.697 degrees, +/−0.698 degrees, +/−0.699 degrees, +/−0.700 degrees, degrees, +/−0.701 degrees, +/−0.702 degrees, +/−0.703 degrees, +/−0.704 degrees, +/−0.705 degrees, +/−0.706 degrees, +/−0.707 degrees, +/−0.708 degrees, +/−0.709 degrees, +/−0.710 degrees, +/−0.711 degrees, +/−0.712 degrees, +/−0.713 degrees, +/−0.714 degrees, +/−0.715 degrees, +/−0.716 degrees, +/−0.717 degrees, +/−0.718 degrees, +/−0.719 degrees, +/−0.720 degrees, +/−0.721 degrees, +/−0.722 degrees, +/−0.723 degrees, +/−0.724 degrees, +/−0.725 degrees, +/−0.726 degrees, +/−0.727 degrees, +/−0.728 degrees, +/−0.729 degrees, +/−0.730 degrees, +/−0.731 degrees, +/−0.732 degrees, +/−0.733 degrees, +/−0.734 degrees, +/−0.735 degrees, +/−0.736 degrees, +/−0.737 degrees, +/−0.738 degrees, +/−0.739 degrees, +/−0.740 degrees, +/−0.741 degrees, +/−0.742 degrees, +/−0.743 degrees, +/−0.744 degrees, +/−0.745 degrees, +/−0.746 degrees, +/−0.747 degrees, +/−0.748 degrees, +/−0.749 degrees, +/−0.750 degrees, +/−0.751 degrees, +/−0.752 degrees, +/−0.753 degrees, +/−0.754 degrees, +/−0.755 degrees, +/−0.756 degrees, +/−0.757 degrees, +/−0.758 degrees, +/−0.759 degrees, +/−0.760 degrees, +/−0.761 degrees, +/−0.762 degrees, +/−0.763 degrees, +/−0.764 degrees, +/−0.765 degrees, +/−0.766 degrees, +/−0.767 degrees, +/−0.768 degrees, +/−0.769 degrees, +/−0.770 degrees, +/−0.771 degrees, +/−0.772 degrees, +/−0.773 degrees, +/−0.774 degrees, +/−0.775 degrees, +/−0.776 degrees, +/−0.777 degrees, +/−0.778 degrees, +/−0.779 degrees, +/−0.780 degrees, +/−0.781 degrees, +/−0.782 degrees, +/−0.783 degrees, +/−0.784 degrees, +/−0.785 degrees, +/−0.786 degrees, +/−0.787 degrees, +/−0.788 degrees, +/−0.789 degrees, +/−0.790 degrees, +/−0.791 degrees, +/−0.792 degrees, +/−0.793 degrees, +/−0.794 degrees, +/−0.795 degrees, +/−0.796 degrees, +/−0.797 degrees, +/−0.798 degrees, +/−0.799 degrees, +/−0.800 degrees, degrees, +/−0.801 degrees, +/−0.802 degrees, +/−0.803 degrees, +/−0.804 degrees, +/−0.805 degrees, +/−0.806 degrees, +/−0.807 degrees, +/−0.808 degrees, +/−0.809 degrees, +/−0.810 degrees, +/−0.811 degrees, +/−0.812 degrees, +/−0.813 degrees, +/−0.814 degrees, +/−0.815 degrees, +/−0.816 degrees, +/−0.817 degrees, +/−0.818 degrees, +/−0.819 degrees, +/−0.820 degrees, +/−0.821 degrees, +/−0.822 degrees, +/−0.823 degrees, +/−0.824 degrees, +/−0.825 degrees, +/−0.826 degrees, +/−0.827 degrees, +/−0.828 degrees, +/−0.829 degrees, +/−0.830 degrees, +/−0.831 degrees, +/−0.832 degrees, +/−0.833 degrees, +/−0.834 degrees, +/−0.835 degrees, +/−0.836 degrees, +/−0.837 degrees, +/−0.838 degrees, +/−0.839 degrees, +/−0.840 degrees, +/−0.841 degrees, +/−0.842 degrees, +/−0.843 degrees, +/−0.844 degrees, +/−0.845 degrees, +/−0.846 degrees, +/−0.847 degrees, +/−0.848 degrees, +/−0.849 degrees, +/−0.850 degrees, +/−0.851 degrees, +/−0.852 degrees, +/−0.853 degrees, +/−0.854 degrees, +/−0.855 degrees, +/−0.856 degrees, +/−0.857 degrees, +/−0.858 degrees, +/−0.859 degrees, +/−0.860 degrees, +/−0.861 degrees, +/−0.862 degrees, +/−0.863 degrees, +/−0.864 degrees, +/−0.865 degrees, +/−0.866 degrees, +/−0.867 degrees, +/−0.868 degrees, +/−0.869 degrees, +/−0.870 degrees, +/−0.871 degrees, +/−0.872 degrees, +/−0.873 degrees, +/−0.874 degrees, +/−0.875 degrees, +/−0.876 degrees, +/−0.877 degrees, +/−0.878 degrees, +/−0.879 degrees, +/−0.880 degrees, +/−0.881 degrees, +/−0.882 degrees, +/−0.883 degrees, +/−0.884 degrees, +/−0.885 degrees, +/−0.886 degrees, +/−0.887 degrees, +/−0.888 degrees, +/−0.889 degrees, +/−0.890 degrees, +/−0.891 degrees, +/−0.892 degrees, +/−0.893 degrees, +/−0.894 degrees, +/−0.895 degrees, +/−0.896 degrees, +/−0.897 degrees, +/−0.898 degrees, +/−0.899 degrees, +/−0.900 degrees, degrees, +/−0.901 degrees, +/−0.902 degrees, +/−0.903 degrees, +/−0.904 degrees, +/−0.905 degrees, +/−0.906 degrees, +/−0.907 degrees, +/−0.908 degrees, +/−0.909 degrees, +/−0.910 degrees, +/−0.911 degrees, +/−0.912 degrees, +/−0.913 degrees, +/−0.914 degrees, +/−0.915 degrees, +/−0.916 degrees, +/−0.917 degrees, +/−0.918 degrees, +/−0.919 degrees, +/−0.920 degrees, +/−0.921 degrees, +/−0.922 degrees, +/−0.923 degrees, +/−0.924 degrees, +/−0.925 degrees, +/−0.926 degrees, +/−0.927 degrees, +/−0.928 degrees, +/−0.929 degrees, +/−0.930 degrees, +/−0.931 degrees, +/−0.932 degrees, +/−0.933 degrees, +/−0.934 degrees, +/−0.935 degrees, +/−0.936 degrees, +/−0.937 degrees, +/−0.938 degrees, +/−0.939 degrees, +/−0.940 degrees, +/−0.941 degrees, +/−0.942 degrees, +/−0.943 degrees, +/−0.944 degrees, +/−0.945 degrees, +/−0.946 degrees, +/−0.947 degrees, +/−0.948 degrees, +/−0.949 degrees, +/−0.950 degrees, +/−0.951 degrees, +/−0.952 degrees, +/−0.953 degrees, +/−0.954 degrees, +/−0.955 degrees, +/−0.956 degrees, +/−0.957 degrees, +/−0.958 degrees, +/−0.959 degrees, +/−0.960 degrees, +/−0.961 degrees, +/−0.962 degrees, +/−0.963 degrees, +/−0.964 degrees, +/−0.965 degrees, +/−0.966 degrees, +/−0.967 degrees, +/−0.968 degrees, +/−0.969 degrees, +/−0.970 degrees, +/−0.971 degrees, +/−0.972 degrees, +/−0.973 degrees, +/−0.974 degrees, +/−0.975 degrees, +/−0.976 degrees, +/−0.977 degrees, +/−0.978 degrees, +/−0.979 degrees, +/−0.980 degrees, +/−0.981 degrees, +/−0.982 degrees, +/−0.983 degrees, +/−0.984 degrees, +/−0.985 degrees, +/−0.986 degrees, +/−0.987 degrees, +/−0.988 degrees, +/−0.989 degrees, +/−0.990 degrees, +/−0.991 degrees, +/−0.992 degrees, +/−0.993 degrees, +/−0.994 degrees, +/−0.995 degrees, +/−0.996 degrees, +/−0.997 degrees, +/−0.998 degrees, +/−0.999 degrees, +/−1.000 degrees, +/−1.100, degrees, +/−1.101 degrees, +/−1.102 degrees, +/−1.103 degrees, +/−1.104 degrees, +/−1.105 degrees, +/−1.106 degrees, +/−1.107 degrees, +/−1.108 degrees, +/−1.109 degrees, +/−1.110 degrees, +/−1.111 degrees, +/−1.112 degrees, +/−1.113 degrees, +/−1.114 degrees, +/−1.115 degrees, +/−1.116 degrees, +/−1.117 degrees, +/−1.118 degrees, +/−1.119 degrees, +/−1.120 degrees, +/−1.121 degrees, +/−1.122 degrees, +/−1.123 degrees, +/−1.124 degrees, +/−1.125 degrees, +/−1.126 degrees, +/−1.127 degrees, +/−1.128 degrees, +/−1.129 degrees, +/−1.130 degrees, +/−1.131 degrees, +/−1.132 degrees, +/−1.133 degrees, +/−1.134 degrees, +/−1.135 degrees, +/−1.136 degrees, +/−1.137 degrees, +/−1.138 degrees, +/−1.139 degrees, +/−1.140 degrees, +/−1.141 degrees, +/−1.142 degrees, +/−1.143 degrees, +/−1.144 degrees, +/−1.145 degrees, +/−1.146 degrees, +/−1.147 degrees, +/−1.148 degrees, +/−1.149 degrees, +/−1.150 degrees, +/−1.151 degrees, +/−1.152 degrees, +/−1.153 degrees, +/−1.154 degrees, +/−1.155 degrees, +/−1.156 degrees, +/−1.157 degrees, +/−1.158 degrees, +/−1.159 degrees, +/−1.160 degrees, +/−1.161 degrees, +/−1.162 degrees, +/−1.163 degrees, +/−1.164 degrees, +/−1.165 degrees, +/−1.166 degrees, +/−1.167 degrees, +/−1.168 degrees, +/−1.169 degrees, +/−1.170 degrees, +/−1.171 degrees, +/−1.172 degrees, +/−1.173 degrees, +/−1.174 degrees, +/−1.175 degrees, +/−1.176 degrees, +/−1.177 degrees, +/−1.178 degrees, +/−1.179 degrees, +/−1.180 degrees, +/−1.181 degrees, +/−1.182 degrees, +/−1.183 degrees, +/−1.184 degrees, +/−1.185 degrees, +/−1.186 degrees, +/−1.187 degrees, +/−1.188 degrees, +/−1.189 degrees, +/−1.190 degrees, +/−1.191 degrees, +/−1.192 degrees, +/−1.193 degrees, +/−1.194 degrees, +/−1.195 degrees, +/−1.196 degrees, +/−1.197 degrees, +/−1.198 degrees, +/−1.199 degrees, +/−1.200 degrees, degrees, +/−1.201 degrees, +/−1.202 degrees, +/−1.203 degrees, +/−1.204 degrees, +/−1.205 degrees, +/−1.206 degrees, +/−1.207 degrees, +/−1.208 degrees, +/−1.209 degrees, +/−1.210 degrees, +/−1.211 degrees, +/−1.212 degrees, +/−1.213 degrees, +/−1.214 degrees, +/−1.215 degrees, +/−1.216 degrees, +/−1.217 degrees, +/−1.218 degrees, +/−1.219 degrees, +/−1.220 degrees, +/−1.221 degrees, +/−1.222 degrees, +/−1.223 degrees, +/−1.224 degrees, +/−1.225 degrees, +/−1.226 degrees, +/−1.227 degrees, +/−1.228 degrees, +/−1.229 degrees, +/−1.230 degrees, +/−1.231 degrees, +/−1.232 degrees, +/−1.233 degrees, +/−1.234 degrees, +/−1.235 degrees, +/−1.236 degrees, +/−1.237 degrees, +/−1.238 degrees, +/−1.239 degrees, +/−1.240 degrees, +/−1.241 degrees, +/−1.242 degrees, +/−1.243 degrees, +/−1.244 degrees, +/−1.245 degrees, +/−1.246 degrees, +/−1.247 degrees, +/−1.248 degrees, +/−1.249 degrees, +/−1.250 degrees, +/−1.251 degrees, +/−1.252 degrees, +/−1.253 degrees, +/−1.254 degrees, +/−1.255 degrees, +/−1.256 degrees, +/−1.257 degrees, +/−1.258 degrees, +/−1.259 degrees, +/−1.260 degrees, +/−1.261 degrees, +/−1.262 degrees, +/−1.263 degrees, +/−1.264 degrees, +/−1.265 degrees, +/−1.266 degrees, +/−1.267 degrees, +/−1.268 degrees, +/−1.269 degrees, +/−1.270 degrees, +/−1.271 degrees, +/−1.272 degrees, +/−1.273 degrees, +/−1.274 degrees, +/−1.275 degrees, +/−1.276 degrees, +/−1.277 degrees, +/−1.278 degrees, +/−1.279 degrees, +/−1.280 degrees, +/−1.281 degrees, +/−1.282 degrees, +/−1.283 degrees, +/−1.284 degrees, +/−1.285 degrees, +/−1.286 degrees, +/−1.287 degrees, +/−1.288 degrees, +/−1.289 degrees, +/−1.290 degrees, +/−1.291 degrees, +/−1.292 degrees, +/−1.293 degrees, +/−1.294 degrees, +/−1.295 degrees, +/−1.296 degrees, +/−1.297 degrees, +/−1.298 degrees, +/−1.299 degrees, +/−1.300 degrees, degrees, +/−1.301 degrees, +/−1.302 degrees, +/−1.303 degrees, +/−1.304 degrees, +/−1.305 degrees, +/−1.306 degrees, +/−1.307 degrees, +/−1.308 degrees, +/−1.309 degrees, +/−1.310 degrees, +/−1.311 degrees, +/−1.312 degrees, +/−1.313 degrees, +/−1.314 degrees, +/−1.315 degrees, +/−1.316 degrees, +/−1.317 degrees, +/−1.318 degrees, +/−1.319 degrees, +/−1.320 degrees, +/−1.321 degrees, +/−1.322 degrees, +/−1.323 degrees, +/−1.324 degrees, +/−1.325 degrees, +/−1.326 degrees, +/−1.327 degrees, +/−1.328 degrees, +/−1.329 degrees, +/−1.330 degrees, +/−1.331 degrees, +/−1.332 degrees, +/−1.333 degrees, +/−1.334 degrees, +/−1.335 degrees, +/−1.336 degrees, +/−1.337 degrees, +/−1.338 degrees, +/−1.339 degrees, +/−1.340 degrees, +/−1.341 degrees, +/−1.342 degrees, +/−1.343 degrees, +/−1.344 degrees, +/−1.345 degrees, +/−1.346 degrees, +/−1.347 degrees, +/−1.348 degrees, +/−1.349 degrees, +/−1.350 degrees, +/−1.351 degrees, +/−1.352 degrees, +/−1.353 degrees, +/−1.354 degrees, +/−1.355 degrees, +/−1.356 degrees, +/−1.357 degrees, +/−1.358 degrees, +/−1.359 degrees, +/−1.360 degrees, +/−1.361 degrees, +/−1.362 degrees, +/−1.363 degrees, +/−1.364 degrees, +/−1.365 degrees, +/−1.366 degrees, +/−1.367 degrees, +/−1.368 degrees, +/−1.369 degrees, +/−1.370 degrees, +/−1.371 degrees, +/−1.372 degrees, +/−1.373 degrees, +/−1.374 degrees, +/−1.375 degrees, +/−1.376 degrees, +/−1.377 degrees, +/−1.378 degrees, +/−1.379 degrees, +/−1.380 degrees, +/−1.381 degrees, +/−1.382 degrees, +/−1.383 degrees, +/−1.384 degrees, +/−1.385 degrees, +/−1.386 degrees, +/−1.387 degrees, +/−1.388 degrees, +/−1.389 degrees, +/−1.390 degrees, +/−1.391 degrees, +/−1.392 degrees, +/−1.393 degrees, +/−1.394 degrees, +/−1.395 degrees, +/−1.396 degrees, +/−1.397 degrees, +/−1.398 degrees, +/−1.399 degrees, +/−1.400 degrees, degrees, +/−1.401 degrees, +/−1.402 degrees, +/−1.403 degrees, +/−1.404 degrees, +/−1.405 degrees, +/−1.406 degrees, +/−1.407 degrees, +/−1.408 degrees, +/−1.409 degrees, +/−1.410 degrees, +/−1.411 degrees, +/−1.412 degrees, +/−1.413 degrees, +/−1.414 degrees, +/−1.415 degrees, +/−1.416 degrees, +/−1.417 degrees, +/−1.418 degrees, +/−1.419 degrees, +/−1.420 degrees, +/−1.421 degrees, +/−1.422 degrees, +/−1.423 degrees, +/−1.424 degrees, +/−1.425 degrees, +/−1.426 degrees, +/−1.427 degrees, +/−1.428 degrees, +/−1.429 degrees, +/−1.430 degrees, +/−1.431 degrees, +/−1.432 degrees, +/−1.433 degrees, +/−1.434 degrees, +/−1.435 degrees, +/−1.436 degrees, +/−1.437 degrees, +/−1.438 degrees, +/−1.439 degrees, +/−1.440 degrees, +/−1.441 degrees, +/−1.442 degrees, +/−1.443 degrees, +/−1.444 degrees, +/−1.445 degrees, +/−1.446 degrees, +/−1.447 degrees, +/−1.448 degrees, +/−1.449 degrees, +/−1.450 degrees, +/−1.451 degrees, +/−1.452 degrees, +/−1.453 degrees, +/−1.454 degrees, +/−1.455 degrees, +/−1.456 degrees, +/−1.457 degrees, +/−1.458 degrees, +/−1.459 degrees, +/−1.460 degrees, +/−1.461 degrees, +/−1.462 degrees, +/−1.463 degrees, +/−1.464 degrees, +/−1.465 degrees, +/−1.466 degrees, +/−1.467 degrees, +/−1.468 degrees, +/−1.469 degrees, +/−1.470 degrees, +/−1.471 degrees, +/−1.472 degrees, +/−1.473 degrees, +/−1.474 degrees, +/−1.475 degrees, +/−1.476 degrees, +/−1.477 degrees, +/−1.478 degrees, +/−1.479 degrees, +/−1.480 degrees, +/−1.481 degrees, +/−1.482 degrees, +/−1.483 degrees, +/−1.484 degrees, +/−1.485 degrees, +/−1.486 degrees, +/−1.487 degrees, +/−1.488 degrees, +/−1.489 degrees, +/−1.490 degrees, +/−1.491 degrees, +/−1.492 degrees, +/−1.493 degrees, +/−1.494 degrees, +/−1.495 degrees, +/−1.496 degrees, +/−1.497 degrees, +/−1.498 degrees, +/−1.499 degrees, +/−1.500 degrees, degrees, +/−1.501 degrees, +/−1.502 degrees, +/−1.503 degrees, +/−1.504 degrees, +/−1.505 degrees, +/−1.506 degrees, +/−1.507 degrees, +/−1.508 degrees, +/−1.509 degrees, +/−1.510 degrees, +/−1.511 degrees, +/−1.512 degrees, +/−1.513 degrees, +/−1.514 degrees, +/−1.515 degrees, +/−1.516 degrees, +/−1.517 degrees, +/−1.518 degrees, +/−1.519 degrees, +/−1.520 degrees, +/−1.521 degrees, +/−1.522 degrees, +/−1.523 degrees, +/−1.524 degrees, +/−1.525 degrees, +/−1.526 degrees, +/−1.527 degrees, +/−1.528 degrees, +/−1.529 degrees, +/−1.530 degrees, +/−1.531 degrees, +/−1.532 degrees, +/−1.533 degrees, +/−1.534 degrees, +/−1.535 degrees, +/−1.536 degrees, +/−1.537 degrees, +/−1.538 degrees, +/−1.539 degrees, +/−1.540 degrees, +/−1.541 degrees, +/−1.542 degrees, +/−1.543 degrees, +/−1.544 degrees, +/−1.545 degrees, +/−1.546 degrees, +/−1.547 degrees, +/−1.548 degrees, +/−1.549 degrees, +/−1.550 degrees, +/−1.551 degrees, +/−1.552 degrees, +/−1.553 degrees, +/−1.554 degrees, +/−1.555 degrees, +/−1.556 degrees, +/−1.557 degrees, +/−1.558 degrees, +/−1.559 degrees, +/−1.560 degrees, +/−1.561 degrees, +/−1.562 degrees, +/−1.563 degrees, +/−1.564 degrees, +/−1.565 degrees, +/−1.566 degrees, +/−1.567 degrees, +/−1.568 degrees, +/−1.569 degrees, +/−1.570 degrees, +/−1.571 degrees, +/−1.572 degrees, +/−1.573 degrees, +/−1.574 degrees, +/−1.575 degrees, +/−1.576 degrees, +/−1.577 degrees, +/−1.578 degrees, +/−1.579 degrees, +/−1.580 degrees, +/−1.581 degrees, +/−1.582 degrees, +/−1.583 degrees, +/−1.584 degrees, +/−1.585 degrees, +/−1.586 degrees, +/−1.587 degrees, +/−1.588 degrees, +/−1.589 degrees, +/−1.590 degrees, +/−1.591 degrees, +/−1.592 degrees, +/−1.593 degrees, +/−1.594 degrees, +/−1.595 degrees, +/−1.596 degrees, +/−1.597 degrees, +/−1.598 degrees, +/−1.599 degrees, +/−1.600 degrees, degrees, +/−1.601 degrees, +/−1.602 degrees, +/−1.603 degrees, +/−1.604 degrees, +/−1.605 degrees, +/−1.606 degrees, +/−1.607 degrees, +/−1.608 degrees, +/−1.609 degrees, +/−1.610 degrees, +/−1.611 degrees, +/−1.612 degrees, +/−1.613 degrees, +/−1.614 degrees, +/−1.615 degrees, +/−1.616 degrees, +/−1.617 degrees, +/−1.618 degrees, +/−1.619 degrees, +/−1.620 degrees, +/−1.621 degrees, +/−1.622 degrees, +/−1.623 degrees, +/−1.624 degrees, +/−1.625 degrees, +/−1.626 degrees, +/−1.627 degrees, +/−1.628 degrees, +/−1.629 degrees, +/−1.630 degrees, +/−1.631 degrees, +/−1.632 degrees, +/−1.633 degrees, +/−1.634 degrees, +/−1.635 degrees, +/−1.636 degrees, +/−1.637 degrees, +/−1.638 degrees, +/−1.639 degrees, +/−1.640 degrees, +/−1.641 degrees, +/−1.642 degrees, +/−1.643 degrees, +/−1.644 degrees, +/−1.645 degrees, +/−1.646 degrees, +/−1.647 degrees, +/−1.648 degrees, +/−1.649 degrees, +/−1.650 degrees, +/−1.651 degrees, +/−1.652 degrees, +/−1.653 degrees, +/−1.654 degrees, +/−1.655 degrees, +/−1.656 degrees, +/−1.657 degrees, +/−1.658 degrees, +/−1.659 degrees, +/−1.660 degrees, +/−1.661 degrees, +/−1.662 degrees, +/−1.663 degrees, +/−1.664 degrees, +/−1.665 degrees, +/−1.666 degrees, +/−1.667 degrees, +/−1.668 degrees, +/−1.669 degrees, +/−1.670 degrees, +/−1.671 degrees, +/−1.672 degrees, +/−1.673 degrees, +/−1.674 degrees, +/−1.675 degrees, +/−1.676 degrees, +/−1.677 degrees, +/−1.678 degrees, +/−1.679 degrees, +/−1.680 degrees, +/−1.681 degrees, +/−1.682 degrees, +/−1.683 degrees, +/−1.684 degrees, +/−1.685 degrees, +/−1.686 degrees, +/−1.687 degrees, +/−1.688 degrees, +/−1.689 degrees, +/−1.690 degrees, +/−1.691 degrees, +/−1.692 degrees, +/−1.693 degrees, +/−1.694 degrees, +/−1.695 degrees, +/−1.696 degrees, +/−1.697 degrees, +/−1.698 degrees, +/−1.699 degrees, +/−1.700 degrees, degrees, +/−1.701 degrees, +/−1.702 degrees, +/−1.703 degrees, +/−1.704 degrees, +/−1.705 degrees, +/−1.706 degrees, +/−1.707 degrees, +/−1.708 degrees, +/−1.709 degrees, +/−1.710 degrees, +/−1.711 degrees, +/−1.712 degrees, +/−1.713 degrees, +/−1.714 degrees, +/−1.715 degrees, +/−1.716 degrees, +/−1.717 degrees, +/−1.718 degrees, +/−1.719 degrees, +/−1.720 degrees, +/−1.721 degrees, +/−1.722 degrees, +/−1.723 degrees, +/−1.724 degrees, +/−1.725 degrees, +/−1.726 degrees, +/−1.727 degrees, +/−1.728 degrees, +/−1.729 degrees, +/−1.730 degrees, +/−1.731 degrees, +/−1.732 degrees, +/−1.733 degrees, +/−1.734 degrees, +/−1.735 degrees, +/−1.736 degrees, +/−1.737 degrees, +/−1.738 degrees, +/−1.739 degrees, +/−1.740 degrees, +/−1.741 degrees, +/−1.742 degrees, +/−1.743 degrees, +/−1.744 degrees, +/−1.745 degrees, +/−1.746 degrees, +/−1.747 degrees, +/−1.748 degrees, +/−1.749 degrees, +/−1.750 degrees, +/−1.751 degrees, +/−1.752 degrees, +/−1.753 degrees, +/−1.754 degrees, +/−1.755 degrees, +/−1.756 degrees, +/−1.757 degrees, +/−1.758 degrees, +/−1.759 degrees, +/−1.760 degrees, +/−1.761 degrees, +/−1.762 degrees, +/−1.763 degrees, +/−1.764 degrees, +/−1.765 degrees, +/−1.766 degrees, +/−1.767 degrees, +/−1.768 degrees, +/−1.769 degrees, +/−1.770 degrees, +/−1.771 degrees, +/−1.772 degrees, +/−1.773 degrees, +/−1.774 degrees, +/−1.775 degrees, +/−1.776 degrees, +/−1.777 degrees, +/−1.778 degrees, +/−1.779 degrees, +/−1.780 degrees, +/−1.781 degrees, +/−1.782 degrees, +/−1.783 degrees, +/−1.784 degrees, +/−1.785 degrees, +/−1.786 degrees, +/−1.787 degrees, +/−1.788 degrees, +/−1.789 degrees, +/−1.790 degrees, +/−1.791 degrees, +/−1.792 degrees, +/−1.793 degrees, +/−1.794 degrees, +/−1.795 degrees, +/−1.796 degrees, +/−1.797 degrees, +/−1.798 degrees, +/−1.799 degrees, +/−1.800 degrees, degrees, +/−1.801 degrees, +/−1.802 degrees, +/−1.803 degrees, +/−1.804 degrees, +/−1.805 degrees, +/−1.806 degrees, +/−1.807 degrees, +/−1.808 degrees, +/−1.809 degrees, +/−1.810 degrees, +/−1.811 degrees, +/−1.812 degrees, +/−1.813 degrees, +/−1.814 degrees, +/−1.815 degrees, +/−1.816 degrees, +/−1.817 degrees, +/−1.818 degrees, +/−1.819 degrees, +/−1.820 degrees, +/−1.821 degrees, +/−1.822 degrees, +/−1.823 degrees, +/−1.824 degrees, +/−1.825 degrees, +/−1.826 degrees, +/−1.827 degrees, +/−1.828 degrees, +/−1.829 degrees, +/−1.830 degrees, +/−1.831 degrees, +/−1.832 degrees, +/−1.833 degrees, +/−1.834 degrees, +/−1.835 degrees, +/−1.836 degrees, +/−1.837 degrees, +/−1.838 degrees, +/−1.839 degrees, +/−1.840 degrees, +/−1.841 degrees, +/−1.842 degrees, +/−1.843 degrees, +/−1.844 degrees, +/−1.845 degrees, +/−1.846 degrees, +/−1.847 degrees, +/−1.848 degrees, +/−1.849 degrees, +/−1.850 degrees, +/−1.851 degrees, +/−1.852 degrees, +/−1.853 degrees, +/−1.854 degrees, +/−1.855 degrees, +/−1.856 degrees, +/−1.857 degrees, +/−1.858 degrees, +/−1.859 degrees, +/−1.860 degrees, +/−1.861 degrees, +/−1.862 degrees, +/−1.863 degrees, +/−1.864 degrees, +/−1.865 degrees, +/−1.866 degrees, +/−1.867 degrees, +/−1.868 degrees, +/−1.869 degrees, +/−1.870 degrees, +/−1.871 degrees, +/−1.872 degrees, +/−1.873 degrees, +/−1.874 degrees, +/−1.875 degrees, +/−1.876 degrees, +/−1.877 degrees, +/−1.878 degrees, +/−1.879 degrees, +/−1.880 degrees, +/−1.881 degrees, +/−1.882 degrees, +/−1.883 degrees, +/−1.884 degrees, +/−1.885 degrees, +/−1.886 degrees, +/−1.887 degrees, +/−1.888 degrees, +/−1.889 degrees, +/−1.890 degrees, +/−1.891 degrees, +/−1.892 degrees, +/−1.893 degrees, +/−1.894 degrees, +/−1.895 degrees, +/−1.896 degrees, +/−1.897 degrees, +/−1.898 degrees, +/−1.899 degrees, +/−1.900 degrees, degrees, +/−1.901 degrees, +/−1.902 degrees, +/−1.903 degrees, +/−1.904 degrees, +/−1.905 degrees, +/−1.906 degrees, +/−1.907 degrees, +/−1.908 degrees, +/−1.909 degrees, +/−1.910 degrees, +/−1.911 degrees, +/−1.912 degrees, +/−1.913 degrees, +/−1.914 degrees, +/−1.915 degrees, +/−1.916 degrees, +/−1.917 degrees, +/−1.918 degrees, +/−1.919 degrees, +/−1.920 degrees, +/−1.921 degrees, +/−1.922 degrees, +/−1.923 degrees, +/−1.924 degrees, +/−1.925 degrees, +/−1.926 degrees, +/−1.927 degrees, +/−1.928 degrees, +/−1.929 degrees, +/−1.930 degrees, +/−1.931 degrees, +/−1.932 degrees, +/−1.933 degrees, +/−1.934 degrees, +/−1.935 degrees, +/−1.936 degrees, +/−1.937 degrees, +/−1.938 degrees, +/−1.939 degrees, +/−1.940 degrees, +/−1.941 degrees, +/−1.942 degrees, +/−1.943 degrees, +/−1.944 degrees, +/−1.945 degrees, +/−1.946 degrees, +/−1.947 degrees, +/−1.948 degrees, +/−1.949 degrees, +/−1.950 degrees, +/−1.951 degrees, +/−1.952 degrees, +/−1.953 degrees, +/−1.954 degrees, +/−1.955 degrees, +/−1.956 degrees, +/−1.957 degrees, +/−1.958 degrees, +/−1.959 degrees, +/−1.960 degrees, +/−1.961 degrees, +/−1.962 degrees, +/−1.963 degrees, +/−1.964 degrees, +/−1.965 degrees, +/−1.966 degrees, +/−1.967 degrees, +/−1.968 degrees, +/−1.969 degrees, +/−1.970 degrees, +/−1.971 degrees, +/−1.972 degrees, +/−1.973 degrees, +/−1.974 degrees, +/−1.975 degrees, +/−1.976 degrees, +/−1.977 degrees, +/−1.978 degrees, +/−1.979 degrees, +/−1.980 degrees, +/−1.981 degrees, +/−1.982 degrees, +/−1.983 degrees, +/−1.984 degrees, +/−1.985 degrees, +/−1.986 degrees, +/−1.987 degrees, +/−1.988 degrees, +/−1.989 degrees, +/−1.990 degrees, +/−1.991 degrees, +/−1.992 degrees, +/−1.993 degrees, +/−1.994 degrees, +/−1.995 degrees, +/−1.996 degrees, +/−1.997 degrees, +/−1.998 degrees, +/−1.999 degrees, +/−2.000 degrees, +/−2.100, degrees, +/−2.101 degrees, +/−2.102 degrees, +/−2.103 degrees, +/−2.104 degrees, +/−2.105 degrees, +/−2.106 degrees, +/−2.107 degrees, +/−2.108 degrees, +/−2.109 degrees, +/−2.110 degrees, +/−2.111 degrees, +/−2.112 degrees, +/−2.113 degrees, +/−2.114 degrees, +/−2.115 degrees, +/−2.116 degrees, +/−2.117 degrees, +/−2.118 degrees, +/−2.119 degrees, +/−2.120 degrees, +/−2.121 degrees, +/−2.122 degrees, +/−2.123 degrees, +/−2.124 degrees, +/−2.125 degrees, +/−2.126 degrees, +/−2.127 degrees, +/−2.128 degrees, +/−2.129 degrees, +/−2.130 degrees, +/−2.131 degrees, +/−2.132 degrees, +/−2.133 degrees, +/−2.134 degrees, +/−2.135 degrees, +/−2.136 degrees, +/−2.137 degrees, +/−2.138 degrees, +/−2.139 degrees, +/−2.140 degrees, +/−2.141 degrees, +/−2.142 degrees, +/−2.143 degrees, +/−2.144 degrees, +/−2.145 degrees, +/−2.146 degrees, +/−2.147 degrees, +/−2.148 degrees, +/−2.149 degrees, +/−2.150 degrees, +/−2.151 degrees, +/−2.152 degrees, +/−2.153 degrees, +/−2.154 degrees, +/−2.155 degrees, +/−2.156 degrees, +/−2.157 degrees, +/−2.158 degrees, +/−2.159 degrees, +/−2.160 degrees, +/−2.161 degrees, +/−2.162 degrees, +/−2.163 degrees, +/−2.164 degrees, +/−2.165 degrees, +/−2.166 degrees, +/−2.167 degrees, +/−2.168 degrees, +/−2.169 degrees, +/−2.170 degrees, +/−2.171 degrees, +/−2.172 degrees, +/−2.173 degrees, +/−2.174 degrees, +/−2.175 degrees, +/−2.176 degrees, +/−2.177 degrees, +/−2.178 degrees, +/−2.179 degrees, +/−2.180 degrees, +/−2.181 degrees, +/−2.182 degrees, +/−2.183 degrees, +/−2.184 degrees, +/−2.185 degrees, +/−2.186 degrees, +/−2.187 degrees, +/−2.188 degrees, +/−2.189 degrees, +/−2.190 degrees, +/−2.191 degrees, +/−2.192 degrees, +/−2.193 degrees, +/−2.194 degrees, +/−2.195 degrees, +/−2.196 degrees, +/−2.197 degrees, +/−2.198 degrees, +/−2.199 degrees, +/−2.200 degrees, degrees, +/−2.201 degrees, +/−2.202 degrees, +/−2.203 degrees, +/−2.204 degrees, +/−2.205 degrees, +/−2.206 degrees, +/−2.207 degrees, +/−2.208 degrees, +/−2.209 degrees, +/−2.210 degrees, +/−2.211 degrees, +/−2.212 degrees, +/−2.213 degrees, +/−2.214 degrees, +/−2.215 degrees, +/−2.216 degrees, +/−2.217 degrees, +/−2.218 degrees, +/−2.219 degrees, +/−2.220 degrees, +/−2.221 degrees, +/−2.222 degrees, +/−2.223 degrees, +/−2.224 degrees, +/−2.225 degrees, +/−2.226 degrees, +/−2.227 degrees, +/−2.228 degrees, +/−2.229 degrees, +/−2.230 degrees, +/−2.231 degrees, +/−2.232 degrees, +/−2.233 degrees, +/−2.234 degrees, +/−2.235 degrees, +/−2.236 degrees, +/−2.237 degrees, +/−2.238 degrees, +/−2.239 degrees, +/−2.240 degrees, +/−2.241 degrees, +/−2.242 degrees, +/−2.243 degrees, +/−2.244 degrees, +/−2.245 degrees, +/−2.246 degrees, +/−2.247 degrees, +/−2.248 degrees, +/−2.249 degrees, +/−2.250 degrees, +/−2.251 degrees, +/−2.252 degrees, +/−2.253 degrees, +/−2.254 degrees, +/−2.255 degrees, +/−2.256 degrees, +/−2.257 degrees, +/−2.258 degrees, +/−2.259 degrees, +/−2.260 degrees, +/−2.261 degrees, +/−2.262 degrees, +/−2.263 degrees, +/−2.264 degrees, +/−2.265 degrees, +/−2.266 degrees, +/−2.267 degrees, +/−2.268 degrees, +/−2.269 degrees, +/−2.270 degrees, +/−2.271 degrees, +/−2.272 degrees, +/−2.273 degrees, +/−2.274 degrees, +/−2.275 degrees, +/−2.276 degrees, +/−2.277 degrees, +/−2.278 degrees, +/−2.279 degrees, +/−2.280 degrees, +/−2.281 degrees, +/−2.282 degrees, +/−2.283 degrees, +/−2.284 degrees, +/−2.285 degrees, +/−2.286 degrees, +/−2.287 degrees, +/−2.288 degrees, +/−2.289 degrees, +/−2.290 degrees, +/−2.291 degrees, +/−2.292 degrees, +/−2.293 degrees, +/−2.294 degrees, +/−2.295 degrees, +/−2.296 degrees, +/−2.297 degrees, +/−2.298 degrees, +/−2.299 degrees, +/−2.300 degrees, degrees, +/−2.301 degrees, +/−2.302 degrees, +/−2.303 degrees, +/−2.304 degrees, +/−2.305 degrees, +/−2.306 degrees, +/−2.307 degrees, +/−2.308 degrees, +/−2.309 degrees, +/−2.310 degrees, +/−2.311 degrees, +/−2.312 degrees, +/−2.313 degrees, +/−2.314 degrees, +/−2.315 degrees, +/−2.316 degrees, +/−2.317 degrees, +/−2.318 degrees, +/−2.319 degrees, +/−2.320 degrees, +/−2.321 degrees, +/−2.322 degrees, +/−2.323 degrees, +/−2.324 degrees, +/−2.325 degrees, +/−2.326 degrees, +/−2.327 degrees, +/−2.328 degrees, +/−2.329 degrees, +/−2.330 degrees, +/−2.331 degrees, +/−2.332 degrees, +/−2.333 degrees, +/−2.334 degrees, +/−2.335 degrees, +/−2.336 degrees, +/−2.337 degrees, +/−2.338 degrees, +/−2.339 degrees, +/−2.340 degrees, +/−2.341 degrees, +/−2.342 degrees, +/−2.343 degrees, +/−2.344 degrees, +/−2.345 degrees, +/−2.346 degrees, +/−2.347 degrees, +/−2.348 degrees, +/−2.349 degrees, +/−2.350 degrees, +/−2.351 degrees, +/−2.352 degrees, +/−2.353 degrees, +/−2.354 degrees, +/−2.355 degrees, +/−2.356 degrees, +/−2.357 degrees, +/−2.358 degrees, +/−2.359 degrees, +/−2.360 degrees, +/−2.361 degrees, +/−2.362 degrees, +/−2.363 degrees, +/−2.364 degrees, +/−2.365 degrees, +/−2.366 degrees, +/−2.367 degrees, +/−2.368 degrees, +/−2.369 degrees, +/−2.370 degrees, +/−2.371 degrees, +/−2.372 degrees, +/−2.373 degrees, +/−2.374 degrees, +/−2.375 degrees, +/−2.376 degrees, +/−2.377 degrees, +/−2.378 degrees, +/−2.379 degrees, +/−2.380 degrees, +/−2.381 degrees, +/−2.382 degrees, +/−2.383 degrees, +/−2.384 degrees, +/−2.385 degrees, +/−2.386 degrees, +/−2.387 degrees, +/−2.388 degrees, +/−2.389 degrees, +/−2.390 degrees, +/−2.391 degrees, +/−2.392 degrees, +/−2.393 degrees, +/−2.394 degrees, +/−2.395 degrees, +/−2.396 degrees, +/−2.397 degrees, +/−2.398 degrees, +/−2.399 degrees, +/−2.400 degrees, degrees, +/−2.401 degrees, +/−2.402 degrees, +/−2.403 degrees, +/−2.404 degrees, +/−2.405 degrees, +/−2.406 degrees, +/−2.407 degrees, +/−2.408 degrees, +/−2.409 degrees, +/−2.410 degrees, +/−2.411 degrees, +/−2.412 degrees, +/−2.413 degrees, +/−2.414 degrees, +/−2.415 degrees, +/−2.416 degrees, +/−2.417 degrees, +/−2.418 degrees, +/−2.419 degrees, +/−2.420 degrees, +/−2.421 degrees, +/−2.422 degrees, +/−2.423 degrees, +/−2.424 degrees, +/−2.425 degrees, +/−2.426 degrees, +/−2.427 degrees, +/−2.428 degrees, +/−2.429 degrees, +/−2.430 degrees, +/−2.431 degrees, +/−2.432 degrees, +/−2.433 degrees, +/−2.434 degrees, +/−2.435 degrees, +/−2.436 degrees, +/−2.437 degrees, +/−2.438 degrees, +/−2.439 degrees, +/−2.440 degrees, +/−2.441 degrees, +/−2.442 degrees, +/−2.443 degrees, +/−2.444 degrees, +/−2.445 degrees, +/−2.446 degrees, +/−2.447 degrees, +/−2.448 degrees, +/−2.449 degrees, +/−2.450 degrees, +/−2.451 degrees, +/−2.452 degrees, +/−2.453 degrees, +/−2.454 degrees, +/−2.455 degrees, +/−2.456 degrees, +/−2.457 degrees, +/−2.458 degrees, +/−2.459 degrees, +/−2.460 degrees, +/−2.461 degrees, +/−2.462 degrees, +/−2.463 degrees, +/−2.464 degrees, +/−2.465 degrees, +/−2.466 degrees, +/−2.467 degrees, +/−2.468 degrees, +/−2.469 degrees, +/−2.470 degrees, +/−2.471 degrees, +/−2.472 degrees, +/−2.473 degrees, +/−2.474 degrees, +/−2.475 degrees, +/−2.476 degrees, +/−2.477 degrees, +/−2.478 degrees, +/−2.479 degrees, +/−2.480 degrees, +/−2.481 degrees, +/−2.482 degrees, +/−2.483 degrees, +/−2.484 degrees, +/−2.485 degrees, +/−2.486 degrees, +/−2.487 degrees, +/−2.488 degrees, +/−2.489 degrees, +/−2.490 degrees, +/−2.491 degrees, +/−2.492 degrees, +/−2.493 degrees, +/−2.494 degrees, +/−2.495 degrees, +/−2.496 degrees, +/−2.497 degrees, +/−2.498 degrees, +/−2.499 degrees, +/−2.500 degrees, degrees, +/−2.501 degrees, +/−2.502 degrees, +/−2.503 degrees, +/−2.504 degrees, +/−2.505 degrees, +/−2.506 degrees, +/−2.507 degrees, +/−2.508 degrees, +/−2.509 degrees, +/−2.510 degrees, +/−2.511 degrees, +/−2.512 degrees, +/−2.513 degrees, +/−2.514 degrees, +/−2.515 degrees, +/−2.516 degrees, +/−2.517 degrees, +/−2.518 degrees, +/−2.519 degrees, +/−2.520 degrees, +/−2.521 degrees, +/−2.522 degrees, +/−2.523 degrees, +/−2.524 degrees, +/−2.525 degrees, +/−2.526 degrees, +/−2.527 degrees, +/−2.528 degrees, +/−2.529 degrees, +/−2.530 degrees, +/−2.531 degrees, +/−2.532 degrees, +/−2.533 degrees, +/−2.534 degrees, +/−2.535 degrees, +/−2.536 degrees, +/−2.537 degrees, +/−2.538 degrees, +/−2.539 degrees, +/−2.540 degrees, +/−2.541 degrees, +/−2.542 degrees, +/−2.543 degrees, +/−2.544 degrees, +/−2.545 degrees, +/−2.546 degrees, +/−2.547 degrees, +/−2.548 degrees, +/−2.549 degrees, +/−2.550 degrees, +/−2.551 degrees, +/−2.552 degrees, +/−2.553 degrees, +/−2.554 degrees, +/−2.555 degrees, +/−2.556 degrees, +/−2.557 degrees, +/−2.558 degrees, +/−2.559 degrees, +/−2.560 degrees, +/−2.561 degrees, +/−2.562 degrees, +/−2.563 degrees, +/−2.564 degrees, +/−2.565 degrees, +/−2.566 degrees, +/−2.567 degrees, +/−2.568 degrees, +/−2.569 degrees, +/−2.570 degrees, +/−2.571 degrees, +/−2.572 degrees, +/−2.573 degrees, +/−2.574 degrees, +/−2.575 degrees, +/−2.576 degrees, +/−2.577 degrees, +/−2.578 degrees, +/−2.579 degrees, +/−2.580 degrees, +/−2.581 degrees, +/−2.582 degrees, +/−2.583 degrees, +/−2.584 degrees, +/−2.585 degrees, +/−2.586 degrees, +/−2.587 degrees, +/−2.588 degrees, +/−2.589 degrees, +/−2.590 degrees, +/−2.591 degrees, +/−2.592 degrees, +/−2.593 degrees, +/−2.594 degrees, +/−2.595 degrees, +/−2.596 degrees, +/−2.597 degrees, +/−2.598 degrees, +/−2.599 degrees, +/−2.600 degrees, degrees, +/−2.601 degrees, +/−2.602 degrees, +/−2.603 degrees, +/−2.604 degrees, +/−2.605 degrees, +/−2.606 degrees, +/−2.607 degrees, +/−2.608 degrees, +/−2.609 degrees, +/−2.610 degrees, +/−2.611 degrees, +/−2.612 degrees, +/−2.613 degrees, +/−2.614 degrees, +/−2.615 degrees, +/−2.616 degrees, +/−2.617 degrees, +/−2.618 degrees, +/−2.619 degrees, +/−2.620 degrees, +/−2.621 degrees, +/−2.622 degrees, +/−2.623 degrees, +/−2.624 degrees, +/−2.625 degrees, +/−2.626 degrees, +/−2.627 degrees, +/−2.628 degrees, +/−2.629 degrees, +/−2.630 degrees, +/−2.631 degrees, +/−2.632 degrees, +/−2.633 degrees, +/−2.634 degrees, +/−2.635 degrees, +/−2.636 degrees, +/−2.637 degrees, +/−2.638 degrees, +/−2.639 degrees, +/−2.640 degrees, +/−2.641 degrees, +/−2.642 degrees, +/−2.643 degrees, +/−2.644 degrees, +/−2.645 degrees, +/−2.646 degrees, +/−2.647 degrees, +/−2.648 degrees, +/−2.649 degrees, +/−2.650 degrees, +/−2.651 degrees, +/−2.652 degrees, +/−2.653 degrees, +/−2.654 degrees, +/−2.655 degrees, +/−2.656 degrees, +/−2.657 degrees, +/−2.658 degrees, +/−2.659 degrees, +/−2.660 degrees, +/−2.661 degrees, +/−2.662 degrees, +/−2.663 degrees, +/−2.664 degrees, +/−2.665 degrees, +/−2.666 degrees, +/−2.667 degrees, +/−2.668 degrees, +/−2.669 degrees, +/−2.670 degrees, +/−2.671 degrees, +/−2.672 degrees, +/−2.673 degrees, +/−2.674 degrees, +/−2.675 degrees, +/−2.676 degrees, +/−2.677 degrees, +/−2.678 degrees, +/−2.679 degrees, +/−2.680 degrees, +/−2.681 degrees, +/−2.682 degrees, +/−2.683 degrees, +/−2.684 degrees, +/−2.685 degrees, +/−2.686 degrees, +/−2.687 degrees, +/−2.688 degrees, +/−2.689 degrees, +/−2.690 degrees, +/−2.691 degrees, +/−2.692 degrees, +/−2.693 degrees, +/−2.694 degrees, +/−2.695 degrees, +/−2.696 degrees, +/−2.697 degrees, +/−2.698 degrees, +/−2.699 degrees, +/−2.700 degrees, degrees, +/−2.701 degrees, +/−2.702 degrees, +/−2.703 degrees, +/−2.704 degrees, +/−2.705 degrees, +/−2.706 degrees, +/−2.707 degrees, +/−2.708 degrees, +/−2.709 degrees, +/−2.710 degrees, +/−2.711 degrees, +/−2.712 degrees, +/−2.713 degrees, +/−2.714 degrees, +/−2.715 degrees, +/−2.716 degrees, +/−2.717 degrees, +/−2.718 degrees, +/−2.719 degrees, +/−2.720 degrees, +/−2.721 degrees, +/−2.722 degrees, +/−2.723 degrees, +/−2.724 degrees, +/−2.725 degrees, +/−2.726 degrees, +/−2.727 degrees, +/−2.728 degrees, +/−2.729 degrees, +/−2.730 degrees, +/−2.731 degrees, +/−2.732 degrees, +/−2.733 degrees, +/−2.734 degrees, +/−2.735 degrees, +/−2.736 degrees, +/−2.737 degrees, +/−2.738 degrees, +/−2.739 degrees, +/−2.740 degrees, +/−2.741 degrees, +/−2.742 degrees, +/−2.743 degrees, +/−2.744 degrees, +/−2.745 degrees, +/−2.746 degrees, +/−2.747 degrees, +/−2.748 degrees, +/−2.749 degrees, +/−2.750 degrees, +/−2.751 degrees, +/−2.752 degrees, +/−2.753 degrees, +/−2.754 degrees, +/−2.755 degrees, +/−2.756 degrees, +/−2.757 degrees, +/−2.758 degrees, +/−2.759 degrees, +/−2.760 degrees, +/−2.761 degrees, +/−2.762 degrees, +/−2.763 degrees, +/−2.764 degrees, +/−2.765 degrees, +/−2.766 degrees, +/−2.767 degrees, +/−2.768 degrees, +/−2.769 degrees, +/−2.770 degrees, +/−2.771 degrees, +/−2.772 degrees, +/−2.773 degrees, +/−2.774 degrees, +/−2.775 degrees, +/−2.776 degrees, +/−2.777 degrees, +/−2.778 degrees, +/−2.779 degrees, +/−2.780 degrees, +/−2.781 degrees, +/−2.782 degrees, +/−2.783 degrees, +/−2.784 degrees, +/−2.785 degrees, +/−2.786 degrees, +/−2.787 degrees, +/−2.788 degrees, +/−2.789 degrees, +/−2.790 degrees, +/−2.791 degrees, +/−2.792 degrees, +/−2.793 degrees, +/−2.794 degrees, +/−2.795 degrees, +/−2.796 degrees, +/−2.797 degrees, +/−2.798 degrees, +/−2.799 degrees, +/−2.800 degrees, degrees, +/−2.801 degrees, +/−2.802 degrees, +/−2.803 degrees, +/−2.804 degrees, +/−2.805 degrees, +/−2.806 degrees, +/−2.807 degrees, +/−2.808 degrees, +/−2.809 degrees, +/−2.810 degrees, +/−2.811 degrees, +/−2.812 degrees, +/−2.813 degrees, +/−2.814 degrees, +/−2.815 degrees, +/−2.816 degrees, +/−2.817 degrees, +/−2.818 degrees, +/−2.819 degrees, +/−2.820 degrees, +/−2.821 degrees, +/−2.822 degrees, +/−2.823 degrees, +/−2.824 degrees, +/−2.825 degrees, +/−2.826 degrees, +/−2.827 degrees, +/−2.828 degrees, +/−2.829 degrees, +/−2.830 degrees, +/−2.831 degrees, +/−2.832 degrees, +/−2.833 degrees, +/−2.834 degrees, +/−2.835 degrees, +/−2.836 degrees, +/−2.837 degrees, +/−2.838 degrees, +/−2.839 degrees, +/−2.840 degrees, +/−2.841 degrees, +/−2.842 degrees, +/−2.843 degrees, +/−2.844 degrees, +/−2.845 degrees, +/−2.846 degrees, +/−2.847 degrees, +/−2.848 degrees, +/−2.849 degrees, +/−2.850 degrees, +/−2.851 degrees, +/−2.852 degrees, +/−2.853 degrees, +/−2.854 degrees, +/−2.855 degrees, +/−2.856 degrees, +/−2.857 degrees, +/−2.858 degrees, +/−2.859 degrees, +/−2.860 degrees, +/−2.861 degrees, +/−2.862 degrees, +/−2.863 degrees, +/−2.864 degrees, +/−2.865 degrees, +/−2.866 degrees, +/−2.867 degrees, +/−2.868 degrees, +/−2.869 degrees, +/−2.870 degrees, +/−2.871 degrees, +/−2.872 degrees, +/−2.873 degrees, +/−2.874 degrees, +/−2.875 degrees, +/−2.876 degrees, +/−2.877 degrees, +/−2.878 degrees, +/−2.879 degrees, +/−2.880 degrees, +/−2.881 degrees, +/−2.882 degrees, +/−2.883 degrees, +/−2.884 degrees, +/−2.885 degrees, +/−2.886 degrees, +/−2.887 degrees, +/−2.888 degrees, +/−2.889 degrees, +/−2.890 degrees, +/−2.891 degrees, +/−2.892 degrees, +/−2.893 degrees, +/−2.894 degrees, +/−2.895 degrees, +/−2.896 degrees, +/−2.897 degrees, +/−2.898 degrees, +/−2.899 degrees, +/−2.900 degrees, degrees, +/−2.901 degrees, +/−2.902 degrees, +/−2.903 degrees, +/−2.904 degrees, +/−2.905 degrees, +/−2.906 degrees, +/−2.907 degrees, +/−2.908 degrees, +/−2.909 degrees, +/−2.910 degrees, +/−2.911 degrees, +/−2.912 degrees, +/−2.913 degrees, +/−2.914 degrees, +/−2.915 degrees, +/−2.916 degrees, +/−2.917 degrees, +/−2.918 degrees, +/−2.919 degrees, +/−2.920 degrees, +/−2.921 degrees, +/−2.922 degrees, +/−2.923 degrees, +/−2.924 degrees, +/−2.925 degrees, +/−2.926 degrees, +/−2.927 degrees, +/−2.928 degrees, +/−2.929 degrees, +/−2.930 degrees, +/−2.931 degrees, +/−2.932 degrees, +/−2.933 degrees, +/−2.934 degrees, +/−2.935 degrees, +/−2.936 degrees, +/−2.937 degrees, +/−2.938 degrees, +/−2.939 degrees, +/−2.940 degrees, +/−2.941 degrees, +/−2.942 degrees, +/−2.943 degrees, +/−2.944 degrees, +/−2.945 degrees, +/−2.946 degrees, +/−2.947 degrees, +/−2.948 degrees, +/−2.949 degrees, +/−2.950 degrees, +/−2.951 degrees, +/−2.952 degrees, +/−2.953 degrees, +/−2.954 degrees, +/−2.955 degrees, +/−2.956 degrees, +/−2.957 degrees, +/−2.958 degrees, +/−2.959 degrees, +/−2.960 degrees, +/−2.961 degrees, +/−2.962 degrees, +/−2.963 degrees, +/−2.964 degrees, +/−2.965 degrees, +/−2.966 degrees, +/−2.967 degrees, +/−2.968 degrees, +/−2.969 degrees, +/−2.970 degrees, +/−2.971 degrees, +/−2.972 degrees, +/−2.973 degrees, +/−2.974 degrees, +/−2.975 degrees, +/−2.976 degrees, +/−2.977 degrees, +/−2.978 degrees, +/−2.979 degrees, +/−2.980 degrees, +/−2.981 degrees, +/−2.982 degrees, +/−2.983 degrees, +/−2.984 degrees, +/−2.985 degrees, +/−2.986 degrees, +/−2.987 degrees, +/−2.988 degrees, +/−2.989 degrees, +/−2.990 degrees, +/−2.991 degrees, +/−2.992 degrees, +/−2.993 degrees, +/−2.994 degrees, +/−2.995 degrees, +/−2.996 degrees, +/−2.997 degrees, +/−2.998 degrees, +/−2.999 degrees, +/−3.000 degrees, +/−3.100, degrees, +/−3.101 degrees, +/−3.102 degrees, +/−3.103 degrees, +/−3.104 degrees, +/−3.105 degrees, +/−3.106 degrees, +/−3.107 degrees, +/−3.108 degrees, +/−3.109 degrees, +/−3.110 degrees, +/−3.111 degrees, +/−3.112 degrees, +/−3.113 degrees, +/−3.114 degrees, +/−3.115 degrees, +/−3.116 degrees, +/−3.117 degrees, +/−3.118 degrees, +/−3.119 degrees, +/−3.120 degrees, +/−3.121 degrees, +/−3.122 degrees, +/−3.123 degrees, +/−3.124 degrees, +/−3.125 degrees, +/−3.126 degrees, +/−3.127 degrees, +/−3.128 degrees, +/−3.129 degrees, +/−3.130 degrees, +/−3.131 degrees, +/−3.132 degrees, +/−3.133 degrees, +/−3.134 degrees, +/−3.135 degrees, +/−3.136 degrees, +/−3.137 degrees, +/−3.138 degrees, +/−3.139 degrees, +/−3.140 degrees, +/−3.141 degrees, +/−3.142 degrees, +/−3.143 degrees, +/−3.144 degrees, +/−3.145 degrees, +/−3.146 degrees, +/−3.147 degrees, +/−3.148 degrees, +/−3.149 degrees, +/−3.150 degrees, +/−3.151 degrees, +/−3.152 degrees, +/−3.153 degrees, +/−3.154 degrees, +/−3.155 degrees, +/−3.156 degrees, +/−3.157 degrees, +/−3.158 degrees, +/−3.159 degrees, +/−3.160 degrees, +/−3.161 degrees, +/−3.162 degrees, +/−3.163 degrees, +/−3.164 degrees, +/−3.165 degrees, +/−3.166 degrees, +/−3.167 degrees, +/−3.168 degrees, +/−3.169 degrees, +/−3.170 degrees, +/−3.171 degrees, +/−3.172 degrees, +/−3.173 degrees, +/−3.174 degrees, +/−3.175 degrees, +/−3.176 degrees, +/−3.177 degrees, +/−3.178 degrees, +/−3.179 degrees, +/−3.180 degrees, +/−3.181 degrees, +/−3.182 degrees, +/−3.183 degrees, +/−3.184 degrees, +/−3.185 degrees, +/−3.186 degrees, +/−3.187 degrees, +/−3.188 degrees, +/−3.189 degrees, +/−3.190 degrees, +/−3.191 degrees, +/−3.192 degrees, +/−3.193 degrees, +/−3.194 degrees, +/−3.195 degrees, +/−3.196 degrees, +/−3.197 degrees, +/−3.198 degrees, +/−3.199 degrees, +/−3.200 degrees, degrees, +/−3.201 degrees, +/−3.202 degrees, +/−3.203 degrees, +/−3.204 degrees, +/−3.205 degrees, +/−3.206 degrees, +/−3.207 degrees, +/−3.208 degrees, +/−3.209 degrees, +/−3.210 degrees, +/−3.211 degrees, +/−3.212 degrees, +/−3.213 degrees, +/−3.214 degrees, +/−3.215 degrees, +/−3.216 degrees, +/−3.217 degrees, +/−3.218 degrees, +/−3.219 degrees, +/−3.220 degrees, +/−3.221 degrees, +/−3.222 degrees, +/−3.223 degrees, +/−3.224 degrees, +/−3.225 degrees, +/−3.226 degrees, +/−3.227 degrees, +/−3.228 degrees, +/−3.229 degrees, +/−3.230 degrees, +/−3.231 degrees, +/−3.232 degrees, +/−3.233 degrees, +/−3.234 degrees, +/−3.235 degrees, +/−3.236 degrees, +/−3.237 degrees, +/−3.238 degrees, +/−3.239 degrees, +/−3.240 degrees, +/−3.241 degrees, +/−3.242 degrees, +/−3.243 degrees, +/−3.244 degrees, +/−3.245 degrees, +/−3.246 degrees, +/−3.247 degrees, +/−3.248 degrees, +/−3.249 degrees, +/−3.250 degrees, +/−3.251 degrees, +/−3.252 degrees, +/−3.253 degrees, +/−3.254 degrees, +/−3.255 degrees, +/−3.256 degrees, +/−3.257 degrees, +/−3.258 degrees, +/−3.259 degrees, +/−3.260 degrees, +/−3.261 degrees, +/−3.262 degrees, +/−3.263 degrees, +/−3.264 degrees, +/−3.265 degrees, +/−3.266 degrees, +/−3.267 degrees, +/−3.268 degrees, +/−3.269 degrees, +/−3.270 degrees, +/−3.271 degrees, +/−3.272 degrees, +/−3.273 degrees, +/−3.274 degrees, +/−3.275 degrees, +/−3.276 degrees, +/−3.277 degrees, +/−3.278 degrees, +/−3.279 degrees, +/−3.280 degrees, +/−3.281 degrees, +/−3.282 degrees, +/−3.283 degrees, +/−3.284 degrees, +/−3.285 degrees, +/−3.286 degrees, +/−3.287 degrees, +/−3.288 degrees, +/−3.289 degrees, +/−3.290 degrees, +/−3.291 degrees, +/−3.292 degrees, +/−3.293 degrees, +/−3.294 degrees, +/−3.295 degrees, +/−3.296 degrees, +/−3.297 degrees, +/−3.298 degrees, +/−3.299 degrees, +/−3.300 degrees, degrees, +/−3.301 degrees, +/−3.302 degrees, +/−3.303 degrees, +/−3.304 degrees, +/−3.305 degrees, +/−3.306 degrees, +/−3.307 degrees, +/−3.308 degrees, +/−3.309 degrees, +/−3.310 degrees, +/−3.311 degrees, +/−3.312 degrees, +/−3.313 degrees, +/−3.314 degrees, +/−3.315 degrees, +/−3.316 degrees, +/−3.317 degrees, +/−3.318 degrees, +/−3.319 degrees, +/−3.320 degrees, +/−3.321 degrees, +/−3.322 degrees, +/−3.323 degrees, +/−3.324 degrees, +/−3.325 degrees, +/−3.326 degrees, +/−3.327 degrees, +/−3.328 degrees, +/−3.329 degrees, +/−3.330 degrees, +/−3.331 degrees, +/−3.332 degrees, +/−3.333 degrees, +/−3.334 degrees, +/−3.335 degrees, +/−3.336 degrees, +/−3.337 degrees, +/−3.338 degrees, +/−3.339 degrees, +/−3.340 degrees, +/−3.341 degrees, +/−3.342 degrees, +/−3.343 degrees, +/−3.344 degrees, +/−3.345 degrees, +/−3.346 degrees, +/−3.347 degrees, +/−3.348 degrees, +/−3.349 degrees, +/−3.350 degrees, +/−3.351 degrees, +/−3.352 degrees, +/−3.353 degrees, +/−3.354 degrees, +/−3.355 degrees, +/−3.356 degrees, +/−3.357 degrees, +/−3.358 degrees, +/−3.359 degrees, +/−3.360 degrees, +/−3.361 degrees, +/−3.362 degrees, +/−3.363 degrees, +/−3.364 degrees, +/−3.365 degrees, +/−3.366 degrees, +/−3.367 degrees, +/−3.368 degrees, +/−3.369 degrees, +/−3.370 degrees, +/−3.371 degrees, +/−3.372 degrees, +/−3.373 degrees, +/−3.374 degrees, +/−3.375 degrees, +/−3.376 degrees, +/−3.377 degrees, +/−3.378 degrees, +/−3.379 degrees, +/−3.380 degrees, +/−3.381 degrees, +/−3.382 degrees, +/−3.383 degrees, +/−3.384 degrees, +/−3.385 degrees, +/−3.386 degrees, +/−3.387 degrees, +/−3.388 degrees, +/−3.389 degrees, +/−3.390 degrees, +/−3.391 degrees, +/−3.392 degrees, +/−3.393 degrees, +/−3.394 degrees, +/−3.395 degrees, +/−3.396 degrees, +/−3.397 degrees, +/−3.398 degrees, +/−3.399 degrees, +/−3.400 degrees, degrees, +/−3.401 degrees, +/−3.402 degrees, +/−3.403 degrees, +/−3.404 degrees, +/−3.405 degrees, +/−3.406 degrees, +/−3.407 degrees, +/−3.408 degrees, +/−3.409 degrees, +/−3.410 degrees, +/−3.411 degrees, +/−3.412 degrees, +/−3.413 degrees, +/−3.414 degrees, +/−3.415 degrees, +/−3.416 degrees, +/−3.417 degrees, +/−3.418 degrees, +/−3.419 degrees, +/−3.420 degrees, +/−3.421 degrees, +/−3.422 degrees, +/−3.423 degrees, +/−3.424 degrees, +/−3.425 degrees, +/−3.426 degrees, +/−3.427 degrees, +/−3.428 degrees, +/−3.429 degrees, +/−3.430 degrees, +/−3.431 degrees, +/−3.432 degrees, +/−3.433 degrees, +/−3.434 degrees, +/−3.435 degrees, +/−3.436 degrees, +/−3.437 degrees, +/−3.438 degrees, +/−3.439 degrees, +/−3.440 degrees, +/−3.441 degrees, +/−3.442 degrees, +/−3.443 degrees, +/−3.444 degrees, +/−3.445 degrees, +/−3.446 degrees, +/−3.447 degrees, +/−3.448 degrees, +/−3.449 degrees, +/−3.450 degrees, +/−3.451 degrees, +/−3.452 degrees, +/−3.453 degrees, +/−3.454 degrees, +/−3.455 degrees, +/−3.456 degrees, +/−3.457 degrees, +/−3.458 degrees, +/−3.459 degrees, +/−3.460 degrees, +/−3.461 degrees, +/−3.462 degrees, +/−3.463 degrees, +/−3.464 degrees, +/−3.465 degrees, +/−3.466 degrees, +/−3.467 degrees, +/−3.468 degrees, +/−3.469 degrees, +/−3.470 degrees, +/−3.471 degrees, +/−3.472 degrees, +/−3.473 degrees, +/−3.474 degrees, +/−3.475 degrees, +/−3.476 degrees, +/−3.477 degrees, +/−3.478 degrees, +/−3.479 degrees, +/−3.480 degrees, +/−3.481 degrees, +/−3.482 degrees, +/−3.483 degrees, +/−3.484 degrees, +/−3.485 degrees, +/−3.486 degrees, +/−3.487 degrees, +/−3.488 degrees, +/−3.489 degrees, +/−3.490 degrees, +/−3.491 degrees, +/−3.492 degrees, +/−3.493 degrees, +/−3.494 degrees, +/−3.495 degrees, +/−3.496 degrees, +/−3.497 degrees, +/−3.498 degrees, +/−3.499 degrees, +/−3.500 degrees, degrees, +/−3.501 degrees, +/−3.502 degrees, +/−3.503 degrees, +/−3.504 degrees, +/−3.505 degrees, +/−3.506 degrees, +/−3.507 degrees, +/−3.508 degrees, +/−3.509 degrees, +/−3.510 degrees, +/−3.511 degrees, +/−3.512 degrees, +/−3.513 degrees, +/−3.514 degrees, +/−3.515 degrees, +/−3.516 degrees, +/−3.517 degrees, +/−3.518 degrees, +/−3.519 degrees, +/−3.520 degrees, +/−3.521 degrees, +/−3.522 degrees, +/−3.523 degrees, +/−3.524 degrees, +/−3.525 degrees, +/−3.526 degrees, +/−3.527 degrees, +/−3.528 degrees, +/−3.529 degrees, +/−3.530 degrees, +/−3.531 degrees, +/−3.532 degrees, +/−3.533 degrees, +/−3.534 degrees, +/−3.535 degrees, +/−3.536 degrees, +/−3.537 degrees, +/−3.538 degrees, +/−3.539 degrees, +/−3.540 degrees, +/−3.541 degrees, +/−3.542 degrees, +/−3.543 degrees, +/−3.544 degrees, +/−3.545 degrees, +/−3.546 degrees, +/−3.547 degrees, +/−3.548 degrees, +/−3.549 degrees, +/−3.550 degrees, +/−3.551 degrees, +/−3.552 degrees, +/−3.553 degrees, +/−3.554 degrees, +/−3.555 degrees, +/−3.556 degrees, +/−3.557 degrees, +/−3.558 degrees, +/−3.559 degrees, +/−3.560 degrees, +/−3.561 degrees, +/−3.562 degrees, +/−3.563 degrees, +/−3.564 degrees, +/−3.565 degrees, +/−3.566 degrees, +/−3.567 degrees, +/−3.568 degrees, +/−3.569 degrees, +/−3.570 degrees, +/−3.571 degrees, +/−3.572 degrees, +/−3.573 degrees, +/−3.574 degrees, +/−3.575 degrees, +/−3.576 degrees, +/−3.577 degrees, +/−3.578 degrees, +/−3.579 degrees, +/−3.580 degrees, +/−3.581 degrees, +/−3.582 degrees, +/−3.583 degrees, +/−3.584 degrees, +/−3.585 degrees, +/−3.586 degrees, +/−3.587 degrees, +/−3.588 degrees, +/−3.589 degrees, +/−3.590 degrees, +/−3.591 degrees, +/−3.592 degrees, +/−3.593 degrees, +/−3.594 degrees, +/−3.595 degrees, +/−3.596 degrees, +/−3.597 degrees, +/−3.598 degrees, +/−3.599 degrees, +/−3.600 degrees, degrees, +/−3.601 degrees, +/−3.602 degrees, +/−3.603 degrees, +/−3.604 degrees, +/−3.605 degrees, +/−3.606 degrees, +/−3.607 degrees, +/−3.608 degrees, +/−3.609 degrees, +/−3.610 degrees, +/−3.611 degrees, +/−3.612 degrees, +/−3.613 degrees, +/−3.614 degrees, +/−3.615 degrees, +/−3.616 degrees, +/−3.617 degrees, +/−3.618 degrees, +/−3.619 degrees, +/−3.620 degrees, +/−3.621 degrees, +/−3.622 degrees, +/−3.623 degrees, +/−3.624 degrees, +/−3.625 degrees, +/−3.626 degrees, +/−3.627 degrees, +/−3.628 degrees, +/−3.629 degrees, +/−3.630 degrees, +/−3.631 degrees, +/−3.632 degrees, +/−3.633 degrees, +/−3.634 degrees, +/−3.635 degrees, +/−3.636 degrees, +/−3.637 degrees, +/−3.638 degrees, +/−3.639 degrees, +/−3.640 degrees, +/−3.641 degrees, +/−3.642 degrees, +/−3.643 degrees, +/−3.644 degrees, +/−3.645 degrees, +/−3.646 degrees, +/−3.647 degrees, +/−3.648 degrees, +/−3.649 degrees, +/−3.650 degrees, +/−3.651 degrees, +/−3.652 degrees, +/−3.653 degrees, +/−3.654 degrees, +/−3.655 degrees, +/−3.656 degrees, +/−3.657 degrees, +/−3.658 degrees, +/−3.659 degrees, +/−3.660 degrees, +/−3.661 degrees, +/−3.662 degrees, +/−3.663 degrees, +/−3.664 degrees, +/−3.665 degrees, +/−3.666 degrees, +/−3.667 degrees, +/−3.668 degrees, +/−3.669 degrees, +/−3.670 degrees, +/−3.671 degrees, +/−3.672 degrees, +/−3.673 degrees, +/−3.674 degrees, +/−3.675 degrees, +/−3.676 degrees, +/−3.677 degrees, +/−3.678 degrees, +/−3.679 degrees, +/−3.680 degrees, +/−3.681 degrees, +/−3.682 degrees, +/−3.683 degrees, +/−3.684 degrees, +/−3.685 degrees, +/−3.686 degrees, +/−3.687 degrees, +/−3.688 degrees, +/−3.689 degrees, +/−3.690 degrees, +/−3.691 degrees, +/−3.692 degrees, +/−3.693 degrees, +/−3.694 degrees, +/−3.695 degrees, +/−3.696 degrees, +/−3.697 degrees, +/−3.698 degrees, +/−3.699 degrees, +/−3.700 degrees, degrees, +/−3.701 degrees, +/−3.702 degrees, +/−3.703 degrees, +/−3.704 degrees, +/−3.705 degrees, +/−3.706 degrees, +/−3.707 degrees, +/−3.708 degrees, +/−3.709 degrees, +/−3.710 degrees, +/−3.711 degrees, +/−3.712 degrees, +/−3.713 degrees, +/−3.714 degrees, +/−3.715 degrees, +/−3.716 degrees, +/−3.717 degrees, +/−3.718 degrees, +/−3.719 degrees, +/−3.720 degrees, +/−3.721 degrees, +/−3.722 degrees, +/−3.723 degrees, +/−3.724 degrees, +/−3.725 degrees, +/−3.726 degrees, +/−3.727 degrees, +/−3.728 degrees, +/−3.729 degrees, +/−3.730 degrees, +/−3.731 degrees, +/−3.732 degrees, +/−3.733 degrees, +/−3.734 degrees, +/−3.735 degrees, +/−3.736 degrees, +/−3.737 degrees, +/−3.738 degrees, +/−3.739 degrees, +/−3.740 degrees, +/−3.741 degrees, +/−3.742 degrees, +/−3.743 degrees, +/−3.744 degrees, +/−3.745 degrees, +/−3.746 degrees, +/−3.747 degrees, +/−3.748 degrees, +/−3.749 degrees, +/−3.750 degrees, +/−3.751 degrees, +/−3.752 degrees, +/−3.753 degrees, +/−3.754 degrees, +/−3.755 degrees, +/−3.756 degrees, +/−3.757 degrees, +/−3.758 degrees, +/−3.759 degrees, +/−3.760 degrees, +/−3.761 degrees, +/−3.762 degrees, +/−3.763 degrees, +/−3.764 degrees, +/−3.765 degrees, +/−3.766 degrees, +/−3.767 degrees, +/−3.768 degrees, +/−3.769 degrees, +/−3.770 degrees, +/−3.771 degrees, +/−3.772 degrees, +/−3.773 degrees, +/−3.774 degrees, +/−3.775 degrees, +/−3.776 degrees, +/−3.777 degrees, +/−3.778 degrees, +/−3.779 degrees, +/−3.780 degrees, +/−3.781 degrees, +/−3.782 degrees, +/−3.783 degrees, +/−3.784 degrees, +/−3.785 degrees, +/−3.786 degrees, +/−3.787 degrees, +/−3.788 degrees, +/−3.789 degrees, +/−3.790 degrees, +/−3.791 degrees, +/−3.792 degrees, +/−3.793 degrees, +/−3.794 degrees, +/−3.795 degrees, +/−3.796 degrees, +/−3.797 degrees, +/−3.798 degrees, +/−3.799 degrees, +/−3.800 degrees, degrees, +/−3.801 degrees, +/−3.802 degrees, +/−3.803 degrees, +/−3.804 degrees, +/−3.805 degrees, +/−3.806 degrees, +/−3.807 degrees, +/−3.808 degrees, +/−3.809 degrees, +/−3.810 degrees, +/−3.811 degrees, +/−3.812 degrees, +/−3.813 degrees, +/−3.814 degrees, +/−3.815 degrees, +/−3.816 degrees, +/−3.817 degrees, +/−3.818 degrees, +/−3.819 degrees, +/−3.820 degrees, +/−3.821 degrees, +/−3.822 degrees, +/−3.823 degrees, +/−3.824 degrees, +/−3.825 degrees, +/−3.826 degrees, +/−3.827 degrees, +/−3.828 degrees, +/−3.829 degrees, +/−3.830 degrees, +/−3.831 degrees, +/−3.832 degrees, +/−3.833 degrees, +/−3.834 degrees, +/−3.835 degrees, +/−3.836 degrees, +/−3.837 degrees, +/−3.838 degrees, +/−3.839 degrees, +/−3.840 degrees, +/−3.841 degrees, +/−3.842 degrees, +/−3.843 degrees, +/−3.844 degrees, +/−3.845 degrees, +/−3.846 degrees, +/−3.847 degrees, +/−3.848 degrees, +/−3.849 degrees, +/−3.850 degrees, +/−3.851 degrees, +/−3.852 degrees, +/−3.853 degrees, +/−3.854 degrees, +/−3.855 degrees, +/−3.856 degrees, +/−3.857 degrees, +/−3.858 degrees, +/−3.859 degrees, +/−3.860 degrees, +/−3.861 degrees, +/−3.862 degrees, +/−3.863 degrees, +/−3.864 degrees, +/−3.865 degrees, +/−3.866 degrees, +/−3.867 degrees, +/−3.868 degrees, +/−3.869 degrees, +/−3.870 degrees, +/−3.871 degrees, +/−3.872 degrees, +/−3.873 degrees, +/−3.874 degrees, +/−3.875 degrees, +/−3.876 degrees, +/−3.877 degrees, +/−3.878 degrees, +/−3.879 degrees, +/−3.880 degrees, +/−3.881 degrees, +/−3.882 degrees, +/−3.883 degrees, +/−3.884 degrees, +/−3.885 degrees, +/−3.886 degrees, +/−3.887 degrees, +/−3.888 degrees, +/−3.889 degrees, +/−3.890 degrees, +/−3.891 degrees, +/−3.892 degrees, +/−3.893 degrees, +/−3.894 degrees, +/−3.895 degrees, +/−3.896 degrees, +/−3.897 degrees, +/−3.898 degrees, +/−3.899 degrees, +/−3.900 degrees, degrees, +/−3.901 degrees, +/−3.902 degrees, +/−3.903 degrees, +/−3.904 degrees, +/−3.905 degrees, +/−3.906 degrees, +/−3.907 degrees, +/−3.908 degrees, +/−3.909 degrees, +/−3.910 degrees, +/−3.911 degrees, +/−3.912 degrees, +/−3.913 degrees, +/−3.914 degrees, +/−3.915 degrees, +/−3.916 degrees, +/−3.917 degrees, +/−3.918 degrees, +/−3.919 degrees, +/−3.920 degrees, +/−3.921 degrees, +/−3.922 degrees, +/−3.923 degrees, +/−3.924 degrees, +/−3.925 degrees, +/−3.926 degrees, +/−3.927 degrees, +/−3.928 degrees, +/−3.929 degrees, +/−3.930 degrees, +/−3.931 degrees, +/−3.932 degrees, +/−3.933 degrees, +/−3.934 degrees, +/−3.935 degrees, +/−3.936 degrees, +/−3.937 degrees, +/−3.938 degrees, +/−3.939 degrees, +/−3.940 degrees, +/−3.941 degrees, +/−3.942 degrees, +/−3.943 degrees, +/−3.944 degrees, +/−3.945 degrees, +/−3.946 degrees, +/−3.947 degrees, +/−3.948 degrees, +/−3.949 degrees, +/−3.950 degrees, +/−3.951 degrees, +/−3.952 degrees, +/−3.953 degrees, +/−3.954 degrees, +/−3.955 degrees, +/−3.956 degrees, +/−3.957 degrees, +/−3.958 degrees, +/−3.959 degrees, +/−3.960 degrees, +/−3.961 degrees, +/−3.962 degrees, +/−3.963 degrees, +/−3.964 degrees, +/−3.965 degrees, +/−3.966 degrees, +/−3.967 degrees, +/−3.968 degrees, +/−3.969 degrees, +/−3.970 degrees, +/−3.971 degrees, +/−3.972 degrees, +/−3.973 degrees, +/−3.974 degrees, +/−3.975 degrees, +/−3.976 degrees, +/−3.977 degrees, +/−3.978 degrees, +/−3.979 degrees, +/−3.980 degrees, +/−3.981 degrees, +/−3.982 degrees, +/−3.983 degrees, +/−3.984 degrees, +/−3.985 degrees, +/−3.986 degrees, +/−3.987 degrees, +/−3.988 degrees, +/−3.989 degrees, +/−3.990 degrees, +/−3.991 degrees, +/−3.992 degrees, +/−3.993 degrees, +/−3.994 degrees, +/−3.995 degrees, +/−3.996 degrees, +/−3.997 degrees, +/−3.998 degrees, +/−3.999 degrees, +/−4.000 degrees, +/−4.100, degrees, +/−4.101 degrees, +/−4.102 degrees, +/−4.103 degrees, +/−4.104 degrees, +/−4.105 degrees, +/−4.106 degrees, +/−4.107 degrees, +/−4.108 degrees, +/−4.109 degrees, +/−4.110 degrees, +/−4.111 degrees, +/−4.112 degrees, +/−4.113 degrees, +/−4.114 degrees, +/−4.115 degrees, +/−4.116 degrees, +/−4.117 degrees, +/−4.118 degrees, +/−4.119 degrees, +/−4.120 degrees, +/−4.121 degrees, +/−4.122 degrees, +/−4.123 degrees, +/−4.124 degrees, +/−4.125 degrees, +/−4.126 degrees, +/−4.127 degrees, +/−4.128 degrees, +/−4.129 degrees, +/−4.130 degrees, +/−4.131 degrees, +/−4.132 degrees, +/−4.133 degrees, +/−4.134 degrees, +/−4.135 degrees, +/−4.136 degrees, +/−4.137 degrees, +/−4.138 degrees, +/−4.139 degrees, +/−4.140 degrees, +/−4.141 degrees, +/−4.142 degrees, +/−4.143 degrees, +/−4.144 degrees, +/−4.145 degrees, +/−4.146 degrees, +/−4.147 degrees, +/−4.148 degrees, +/−4.149 degrees, +/−4.150 degrees, +/−4.151 degrees, +/−4.152 degrees, +/−4.153 degrees, +/−4.154 degrees, +/−4.155 degrees, +/−4.156 degrees, +/−4.157 degrees, +/−4.158 degrees, +/−4.159 degrees, +/−4.160 degrees, +/−4.161 degrees, +/−4.162 degrees, +/−4.163 degrees, +/−4.164 degrees, +/−4.165 degrees, +/−4.166 degrees, +/−4.167 degrees, +/−4.168 degrees, +/−4.169 degrees, +/−4.170 degrees, +/−4.171 degrees, +/−4.172 degrees, +/−4.173 degrees, +/−4.174 degrees, +/−4.175 degrees, +/−4.176 degrees, +/−4.177 degrees, +/−4.178 degrees, +/−4.179 degrees, +/−4.180 degrees, +/−4.181 degrees, +/−4.182 degrees, +/−4.183 degrees, +/−4.184 degrees, +/−4.185 degrees, +/−4.186 degrees, +/−4.187 degrees, +/−4.188 degrees, +/−4.189 degrees, +/−4.190 degrees, +/−4.191 degrees, +/−4.192 degrees, +/−4.193 degrees, +/−4.194 degrees, +/−4.195 degrees, +/−4.196 degrees, +/−4.197 degrees, +/−4.198 degrees, +/−4.199 degrees, +/−4.200 degrees, degrees, +/−4.201 degrees, +/−4.202 degrees, +/−4.203 degrees, +/−4.204 degrees, +/−4.205 degrees, +/−4.206 degrees, +/−4.207 degrees, +/−4.208 degrees, +/−4.209 degrees, +/−4.210 degrees, +/−4.211 degrees, +/−4.212 degrees, +/−4.213 degrees, +/−4.214 degrees, +/−4.215 degrees, +/−4.216 degrees, +/−4.217 degrees, +/−4.218 degrees, +/−4.219 degrees, +/−4.220 degrees, +/−4.221 degrees, +/−4.222 degrees, +/−4.223 degrees, +/−4.224 degrees, +/−4.225 degrees, +/−4.226 degrees, +/−4.227 degrees, +/−4.228 degrees, +/−4.229 degrees, +/−4.230 degrees, +/−4.231 degrees, +/−4.232 degrees, +/−4.233 degrees, +/−4.234 degrees, +/−4.235 degrees, +/−4.236 degrees, +/−4.237 degrees, +/−4.238 degrees, +/−4.239 degrees, +/−4.240 degrees, +/−4.241 degrees, +/−4.242 degrees, +/−4.243 degrees, +/−4.244 degrees, +/−4.245 degrees, +/−4.246 degrees, +/−4.247 degrees, +/−4.248 degrees, +/−4.249 degrees, +/−4.250 degrees, +/−4.251 degrees, +/−4.252 degrees, +/−4.253 degrees, +/−4.254 degrees, +/−4.255 degrees, +/−4.256 degrees, +/−4.257 degrees, +/−4.258 degrees, +/−4.259 degrees, +/−4.260 degrees, +/−4.261 degrees, +/−4.262 degrees, +/−4.263 degrees, +/−4.264 degrees, +/−4.265 degrees, +/−4.266 degrees, +/−4.267 degrees, +/−4.268 degrees, +/−4.269 degrees, +/−4.270 degrees, +/−4.271 degrees, +/−4.272 degrees, +/−4.273 degrees, +/−4.274 degrees, +/−4.275 degrees, +/−4.276 degrees, +/−4.277 degrees, +/−4.278 degrees, +/−4.279 degrees, +/−4.280 degrees, +/−4.281 degrees, +/−4.282 degrees, +/−4.283 degrees, +/−4.284 degrees, +/−4.285 degrees, +/−4.286 degrees, +/−4.287 degrees, +/−4.288 degrees, +/−4.289 degrees, +/−4.290 degrees, +/−4.291 degrees, +/−4.292 degrees, +/−4.293 degrees, +/−4.294 degrees, +/−4.295 degrees, +/−4.296 degrees, +/−4.297 degrees, +/−4.298 degrees, +/−4.299 degrees, +/−4.300 degrees, degrees, +/−4.301 degrees, +/−4.302 degrees, +/−4.303 degrees, +/−4.304 degrees, +/−4.305 degrees, +/−4.306 degrees, +/−4.307 degrees, +/−4.308 degrees, +/−4.309 degrees, +/−4.310 degrees, +/−4.311 degrees, +/−4.312 degrees, +/−4.313 degrees, +/−4.314 degrees, +/−4.315 degrees, +/−4.316 degrees, +/−4.317 degrees, +/−4.318 degrees, +/−4.319 degrees, +/−4.320 degrees, +/−4.321 degrees, +/−4.322 degrees, +/−4.323 degrees, +/−4.324 degrees, +/−4.325 degrees, +/−4.326 degrees, +/−4.327 degrees, +/−4.328 degrees, +/−4.329 degrees, +/−4.330 degrees, +/−4.331 degrees, +/−4.332 degrees, +/−4.333 degrees, +/−4.334 degrees, +/−4.335 degrees, +/−4.336 degrees, +/−4.337 degrees, +/−4.338 degrees, +/−4.339 degrees, +/−4.340 degrees, +/−4.341 degrees, +/−4.342 degrees, +/−4.343 degrees, +/−4.344 degrees, +/−4.345 degrees, +/−4.346 degrees, +/−4.347 degrees, +/−4.348 degrees, +/−4.349 degrees, +/−4.350 degrees, +/−4.351 degrees, +/−4.352 degrees, +/−4.353 degrees, +/−4.354 degrees, +/−4.355 degrees, +/−4.356 degrees, +/−4.357 degrees, +/−4.358 degrees, +/−4.359 degrees, +/−4.360 degrees, +/−4.361 degrees, +/−4.362 degrees, +/−4.363 degrees, +/−4.364 degrees, +/−4.365 degrees, +/−4.366 degrees, +/−4.367 degrees, +/−4.368 degrees, +/−4.369 degrees, +/−4.370 degrees, +/−4.371 degrees, +/−4.372 degrees, +/−4.373 degrees, +/−4.374 degrees, +/−4.375 degrees, +/−4.376 degrees, +/−4.377 degrees, +/−4.378 degrees, +/−4.379 degrees, +/−4.380 degrees, +/−4.381 degrees, +/−4.382 degrees, +/−4.383 degrees, +/−4.384 degrees, +/−4.385 degrees, +/−4.386 degrees, +/−4.387 degrees, +/−4.388 degrees, +/−4.389 degrees, +/−4.390 degrees, +/−4.391 degrees, +/−4.392 degrees, +/−4.393 degrees, +/−4.394 degrees, +/−4.395 degrees, +/−4.396 degrees, +/−4.397 degrees, +/−4.398 degrees, +/−4.399 degrees, +/−4.400 degrees, degrees, +/−4.401 degrees, +/−4.402 degrees, +/−4.403 degrees, +/−4.404 degrees, +/−4.405 degrees, +/−4.406 degrees, +/−4.407 degrees, +/−4.408 degrees, +/−4.409 degrees, +/−4.410 degrees, +/−4.411 degrees, +/−4.412 degrees, +/−4.413 degrees, +/−4.414 degrees, +/−4.415 degrees, +/−4.416 degrees, +/−4.417 degrees, +/−4.418 degrees, +/−4.419 degrees, +/−4.420 degrees, +/−4.421 degrees, +/−4.422 degrees, +/−4.423 degrees, +/−4.424 degrees, +/−4.425 degrees, +/−4.426 degrees, +/−4.427 degrees, +/−4.428 degrees, +/−4.429 degrees, +/−4.430 degrees, +/−4.431 degrees, +/−4.432 degrees, +/−4.433 degrees, +/−4.434 degrees, +/−4.435 degrees, +/−4.436 degrees, +/−4.437 degrees, +/−4.438 degrees, +/−4.439 degrees, +/−4.440 degrees, +/−4.441 degrees, +/−4.442 degrees, +/−4.443 degrees, +/−4.444 degrees, +/−4.445 degrees, +/−4.446 degrees, +/−4.447 degrees, +/−4.448 degrees, +/−4.449 degrees, +/−4.450 degrees, +/−4.451 degrees, +/−4.452 degrees, +/−4.453 degrees, +/−4.454 degrees, +/−4.455 degrees, +/−4.456 degrees, +/−4.457 degrees, +/−4.458 degrees, +/−4.459 degrees, +/−4.460 degrees, +/−4.461 degrees, +/−4.462 degrees, +/−4.463 degrees, +/−4.464 degrees, +/−4.465 degrees, +/−4.466 degrees, +/−4.467 degrees, +/−4.468 degrees, +/−4.469 degrees, +/−4.470 degrees, +/−4.471 degrees, +/−4.472 degrees, +/−4.473 degrees, +/−4.474 degrees, +/−4.475 degrees, +/−4.476 degrees, +/−4.477 degrees, +/−4.478 degrees, +/−4.479 degrees, +/−4.480 degrees, +/−4.481 degrees, +/−4.482 degrees, +/−4.483 degrees, +/−4.484 degrees, +/−4.485 degrees, +/−4.486 degrees, +/−4.487 degrees, +/−4.488 degrees, +/−4.489 degrees, +/−4.490 degrees, +/−4.491 degrees, +/−4.492 degrees, +/−4.493 degrees, +/−4.494 degrees, +/−4.495 degrees, +/−4.496 degrees, +/−4.497 degrees, +/−4.498 degrees, +/−4.499 degrees, +/−4.500 degrees, degrees, +/−4.501 degrees, +/−4.502 degrees, +/−4.503 degrees, +/−4.504 degrees, +/−4.505 degrees, +/−4.506 degrees, +/−4.507 degrees, +/−4.508 degrees, +/−4.509 degrees, +/−4.510 degrees, +/−4.511 degrees, +/−4.512 degrees, +/−4.513 degrees, +/−4.514 degrees, +/−4.515 degrees, +/−4.516 degrees, +/−4.517 degrees, +/−4.518 degrees, +/−4.519 degrees, +/−4.520 degrees, +/−4.521 degrees, +/−4.522 degrees, +/−4.523 degrees, +/−4.524 degrees, +/−4.525 degrees, +/−4.526 degrees, +/−4.527 degrees, +/−4.528 degrees, +/−4.529 degrees, +/−4.530 degrees, +/−4.531 degrees, +/−4.532 degrees, +/−4.533 degrees, +/−4.534 degrees, +/−4.535 degrees, +/−4.536 degrees, +/−4.537 degrees, +/−4.538 degrees, +/−4.539 degrees, +/−4.540 degrees, +/−4.541 degrees, +/−4.542 degrees, +/−4.543 degrees, +/−4.544 degrees, +/−4.545 degrees, +/−4.546 degrees, +/−4.547 degrees, +/−4.548 degrees, +/−4.549 degrees, +/−4.550 degrees, +/−4.551 degrees, +/−4.552 degrees, +/−4.553 degrees, +/−4.554 degrees, +/−4.555 degrees, +/−4.556 degrees, +/−4.557 degrees, +/−4.558 degrees, +/−4.559 degrees, +/−4.560 degrees, +/−4.561 degrees, +/−4.562 degrees, +/−4.563 degrees, +/−4.564 degrees, +/−4.565 degrees, +/−4.566 degrees, +/−4.567 degrees, +/−4.568 degrees, +/−4.569 degrees, +/−4.570 degrees, +/−4.571 degrees, +/−4.572 degrees, +/−4.573 degrees, +/−4.574 degrees, +/−4.575 degrees, +/−4.576 degrees, +/−4.577 degrees, +/−4.578 degrees, +/−4.579 degrees, +/−4.580 degrees, +/−4.581 degrees, +/−4.582 degrees, +/−4.583 degrees, +/−4.584 degrees, +/−4.585 degrees, +/−4.586 degrees, +/−4.587 degrees, +/−4.588 degrees, +/−4.589 degrees, +/−4.590 degrees, +/−4.591 degrees, +/−4.592 degrees, +/−4.593 degrees, +/−4.594 degrees, +/−4.595 degrees, +/−4.596 degrees, +/−4.597 degrees, +/−4.598 degrees, +/−4.599 degrees, +/−4.600 degrees, degrees, +/−4.601 degrees, +/−4.602 degrees, +/−4.603 degrees, +/−4.604 degrees, +/−4.605 degrees, +/−4.606 degrees, +/−4.607 degrees, +/−4.608 degrees, +/−4.609 degrees, +/−4.610 degrees, +/−4.611 degrees, +/−4.612 degrees, +/−4.613 degrees, +/−4.614 degrees, +/−4.615 degrees, +/−4.616 degrees, +/−4.617 degrees, +/−4.618 degrees, +/−4.619 degrees, +/−4.620 degrees, +/−4.621 degrees, +/−4.622 degrees, +/−4.623 degrees, +/−4.624 degrees, +/−4.625 degrees, +/−4.626 degrees, +/−4.627 degrees, +/−4.628 degrees, +/−4.629 degrees, +/−4.630 degrees, +/−4.631 degrees, +/−4.632 degrees, +/−4.633 degrees, +/−4.634 degrees, +/−4.635 degrees, +/−4.636 degrees, +/−4.637 degrees, +/−4.638 degrees, +/−4.639 degrees, +/−4.640 degrees, +/−4.641 degrees, +/−4.642 degrees, +/−4.643 degrees, +/−4.644 degrees, +/−4.645 degrees, +/−4.646 degrees, +/−4.647 degrees, +/−4.648 degrees, +/−4.649 degrees, +/−4.650 degrees, +/−4.651 degrees, +/−4.652 degrees, +/−4.653 degrees, +/−4.654 degrees, +/−4.655 degrees, +/−4.656 degrees, +/−4.657 degrees, +/−4.658 degrees, +/−4.659 degrees, +/−4.660 degrees, +/−4.661 degrees, +/−4.662 degrees, +/−4.663 degrees, +/−4.664 degrees, +/−4.665 degrees, +/−4.666 degrees, +/−4.667 degrees, +/−4.668 degrees, +/−4.669 degrees, +/−4.670 degrees, +/−4.671 degrees, +/−4.672 degrees, +/−4.673 degrees, +/−4.674 degrees, +/−4.675 degrees, +/−4.676 degrees, +/−4.677 degrees, +/−4.678 degrees, +/−4.679 degrees, +/−4.680 degrees, +/−4.681 degrees, +/−4.682 degrees, +/−4.683 degrees, +/−4.684 degrees, +/−4.685 degrees, +/−4.686 degrees, +/−4.687 degrees, +/−4.688 degrees, +/−4.689 degrees, +/−4.690 degrees, +/−4.691 degrees, +/−4.692 degrees, +/−4.693 degrees, +/−4.694 degrees, +/−4.695 degrees, +/−4.696 degrees, +/−4.697 degrees, +/−4.698 degrees, +/−4.699 degrees, +/−4.700 degrees, degrees, +/−4.701 degrees, +/−4.702 degrees, +/−4.703 degrees, +/−4.704 degrees, +/−4.705 degrees, +/−4.706 degrees, +/−4.707 degrees, +/−4.708 degrees, +/−4.709 degrees, +/−4.710 degrees, +/−4.711 degrees, +/−4.712 degrees, +/−4.713 degrees, +/−4.714 degrees, +/−4.715 degrees, +/−4.716 degrees, +/−4.717 degrees, +/−4.718 degrees, +/−4.719 degrees, +/−4.720 degrees, +/−4.721 degrees, +/−4.722 degrees, +/−4.723 degrees, +/−4.724 degrees, +/−4.725 degrees, +/−4.726 degrees, +/−4.727 degrees, +/−4.728 degrees, +/−4.729 degrees, +/−4.730 degrees, +/−4.731 degrees, +/−4.732 degrees, +/−4.733 degrees, +/−4.734 degrees, +/−4.735 degrees, +/−4.736 degrees, +/−4.737 degrees, +/−4.738 degrees, +/−4.739 degrees, +/−4.740 degrees, +/−4.741 degrees, +/−4.742 degrees, +/−4.743 degrees, +/−4.744 degrees, +/−4.745 degrees, +/−4.746 degrees, +/−4.747 degrees, +/−4.748 degrees, +/−4.749 degrees, +/−4.750 degrees, +/−4.751 degrees, +/−4.752 degrees, +/−4.753 degrees, +/−4.754 degrees, +/−4.755 degrees, +/−4.756 degrees, +/−4.757 degrees, +/−4.758 degrees, +/−4.759 degrees, +/−4.760 degrees, +/−4.761 degrees, +/−4.762 degrees, +/−4.763 degrees, +/−4.764 degrees, +/−4.765 degrees, +/−4.766 degrees, +/−4.767 degrees, +/−4.768 degrees, +/−4.769 degrees, +/−4.770 degrees, +/−4.771 degrees, +/−4.772 degrees, +/−4.773 degrees, +/−4.774 degrees, +/−4.775 degrees, +/−4.776 degrees, +/−4.777 degrees, +/−4.778 degrees, +/−4.779 degrees, +/−4.780 degrees, +/−4.781 degrees, +/−4.782 degrees, +/−4.783 degrees, +/−4.784 degrees, +/−4.785 degrees, +/−4.786 degrees, +/−4.787 degrees, +/−4.788 degrees, +/−4.789 degrees, +/−4.790 degrees, +/−4.791 degrees, +/−4.792 degrees, +/−4.793 degrees, +/−4.794 degrees, +/−4.795 degrees, +/−4.796 degrees, +/−4.797 degrees, +/−4.798 degrees, +/−4.799 degrees, +/−4.800 degrees, degrees, +/−4.801 degrees, +/−4.802 degrees, +/−4.803 degrees, +/−4.804 degrees, +/−4.805 degrees, +/−4.806 degrees, +/−4.807 degrees, +/−4.808 degrees, +/−4.809 degrees, +/−4.810 degrees, +/−4.811 degrees, +/−4.812 degrees, +/−4.813 degrees, +/−4.814 degrees, +/−4.815 degrees, +/−4.816 degrees, +/−4.817 degrees, +/−4.818 degrees, +/−4.819 degrees, +/−4.820 degrees, +/−4.821 degrees, +/−4.822 degrees, +/−4.823 degrees, +/−4.824 degrees, +/−4.825 degrees, +/−4.826 degrees, +/−4.827 degrees, +/−4.828 degrees, +/−4.829 degrees, +/−4.830 degrees, +/−4.831 degrees, +/−4.832 degrees, +/−4.833 degrees, +/−4.834 degrees, +/−4.835 degrees, +/−4.836 degrees, +/−4.837 degrees, +/−4.838 degrees, +/−4.839 degrees, +/−4.840 degrees, +/−4.841 degrees, +/−4.842 degrees, +/−4.843 degrees, +/−4.844 degrees, +/−4.845 degrees, +/−4.846 degrees, +/−4.847 degrees, +/−4.848 degrees, +/−4.849 degrees, +/−4.850 degrees, +/−4.851 degrees, +/−4.852 degrees, +/−4.853 degrees, +/−4.854 degrees, +/−4.855 degrees, +/−4.856 degrees, +/−4.857 degrees, +/−4.858 degrees, +/−4.859 degrees, +/−4.860 degrees, +/−4.861 degrees, +/−4.862 degrees, +/−4.863 degrees, +/−4.864 degrees, +/−4.865 degrees, +/−4.866 degrees, +/−4.867 degrees, +/−4.868 degrees, +/−4.869 degrees, +/−4.870 degrees, +/−4.871 degrees, +/−4.872 degrees, +/−4.873 degrees, +/−4.874 degrees, +/−4.875 degrees, +/−4.876 degrees, +/−4.877 degrees, +/−4.878 degrees, +/−4.879 degrees, +/−4.880 degrees, +/−4.881 degrees, +/−4.882 degrees, +/−4.883 degrees, +/−4.884 degrees, +/−4.885 degrees, +/−4.886 degrees, +/−4.887 degrees, +/−4.888 degrees, +/−4.889 degrees, +/−4.890 degrees, +/−4.891 degrees, +/−4.892 degrees, +/−4.893 degrees, +/−4.894 degrees, +/−4.895 degrees, +/−4.896 degrees, +/−4.897 degrees, +/−4.898 degrees, +/−4.899 degrees, +/−4.900 degrees, degrees, +/−4.901 degrees, +/−4.902 degrees, +/−4.903 degrees, +/−4.904 degrees, +/−4.905 degrees, +/−4.906 degrees, +/−4.907 degrees, +/−4.908 degrees, +/−4.909 degrees, +/−4.910 degrees, +/−4.911 degrees, +/−4.912 degrees, +/−4.913 degrees, +/−4.914 degrees, +/−4.915 degrees, +/−4.916 degrees, +/−4.917 degrees, +/−4.918 degrees, +/−4.919 degrees, +/−4.920 degrees, +/−4.921 degrees, +/−4.922 degrees, +/−4.923 degrees, +/−4.924 degrees, +/−4.925 degrees, +/−4.926 degrees, +/−4.927 degrees, +/−4.928 degrees, +/−4.929 degrees, +/−4.930 degrees, +/−4.931 degrees, +/−4.932 degrees, +/−4.933 degrees, +/−4.934 degrees, +/−4.935 degrees, +/−4.936 degrees, +/−4.937 degrees, +/−4.938 degrees, +/−4.939 degrees, +/−4.940 degrees, +/−4.941 degrees, +/−4.942 degrees, +/−4.943 degrees, +/−4.944 degrees, +/−4.945 degrees, +/−4.946 degrees, +/−4.947 degrees, +/−4.948 degrees, +/−4.949 degrees, +/−4.950 degrees, +/−4.951 degrees, +/−4.952 degrees, +/−4.953 degrees, +/−4.954 degrees, +/−4.955 degrees, +/−4.956 degrees, +/−4.957 degrees, +/−4.958 degrees, +/−4.959 degrees, +/−4.960 degrees, +/−4.961 degrees, +/−4.962 degrees, +/−4.963 degrees, +/−4.964 degrees, +/−4.965 degrees, +/−4.966 degrees, +/−4.967 degrees, +/−4.968 degrees, +/−4.969 degrees, +/−4.970 degrees, +/−4.971 degrees, +/−4.972 degrees, +/−4.973 degrees, +/−4.974 degrees, +/−4.975 degrees, +/−4.976 degrees, +/−4.977 degrees, +/−4.978 degrees, +/−4.979 degrees, +/−4.980 degrees, +/−4.981 degrees, +/−4.982 degrees, +/−4.983 degrees, +/−4.984 degrees, +/−4.985 degrees, +/−4.986 degrees, +/−4.987 degrees, +/−4.988 degrees, +/−4.989 degrees, +/−4.990 degrees, +/−4.991 degrees, +/−4.992 degrees, +/−4.993 degrees, +/−4.994 degrees, +/−4.995 degrees, +/−4.996 degrees, +/−4.997 degrees, +/−4.998 degrees, +/−4.999 degrees, +/−5.000 degrees, +/−5.100, degrees, +/−5.101 degrees, +/−5.102 degrees, +/−5.103 degrees, +/−5.104 degrees, +/−5.105 degrees, +/−5.106 degrees, +/−5.107 degrees, +/−5.108 degrees, +/−5.109 degrees, +/−5.110 degrees, +/−5.111 degrees, +/−5.112 degrees, +/−5.113 degrees, +/−5.114 degrees, +/−5.115 degrees, +/−5.116 degrees, +/−5.117 degrees, +/−5.118 degrees, +/−5.119 degrees, +/−5.120 degrees, +/−5.121 degrees, +/−5.122 degrees, +/−5.123 degrees, +/−5.124 degrees, +/−5.125 degrees, +/−5.126 degrees, +/−5.127 degrees, +/−5.128 degrees, +/−5.129 degrees, +/−5.130 degrees, +/−5.131 degrees, +/−5.132 degrees, +/−5.133 degrees, +/−5.134 degrees, +/−5.135 degrees, +/−5.136 degrees, +/−5.137 degrees, +/−5.138 degrees, +/−5.139 degrees, +/−5.140 degrees, +/−5.141 degrees, +/−5.142 degrees, +/−5.143 degrees, +/−5.144 degrees, +/−5.145 degrees, +/−5.146 degrees, +/−5.147 degrees, +/−5.148 degrees, +/−5.149 degrees, +/−5.150 degrees, +/−5.151 degrees, +/−5.152 degrees, +/−5.153 degrees, +/−5.154 degrees, +/−5.155 degrees, +/−5.156 degrees, +/−5.157 degrees, +/−5.158 degrees, +/−5.159 degrees, +/−5.160 degrees, +/−5.161 degrees, +/−5.162 degrees, +/−5.163 degrees, +/−5.164 degrees, +/−5.165 degrees, +/−5.166 degrees, +/−5.167 degrees, +/−5.168 degrees, +/−5.169 degrees, +/−5.170 degrees, +/−5.171 degrees, +/−5.172 degrees, +/−5.173 degrees, +/−5.174 degrees, +/−5.175 degrees, +/−5.176 degrees, +/−5.177 degrees, +/−5.178 degrees, +/−5.179 degrees, +/−5.180 degrees, +/−5.181 degrees, +/−5.182 degrees, +/−5.183 degrees, +/−5.184 degrees, +/−5.185 degrees, +/−5.186 degrees, +/−5.187 degrees, +/−5.188 degrees, +/−5.189 degrees, +/−5.190 degrees, +/−5.191 degrees, +/−5.192 degrees, +/−5.193 degrees, +/−5.194 degrees, +/−5.195 degrees, +/−5.196 degrees, +/−5.197 degrees, +/−5.198 degrees, +/−5.199 degrees, +/−5.200 degrees, degrees, +/−5.201 degrees, +/−5.202 degrees, +/−5.203 degrees, +/−5.204 degrees, +/−5.205 degrees, +/−5.206 degrees, +/−5.207 degrees, +/−5.208 degrees, +/−5.209 degrees, +/−5.210 degrees, +/−5.211 degrees, +/−5.212 degrees, +/−5.213 degrees, +/−5.214 degrees, +/−5.215 degrees, +/−5.216 degrees, +/−5.217 degrees, +/−5.218 degrees, +/−5.219 degrees, +/−5.220 degrees, +/−5.221 degrees, +/−5.222 degrees, +/−5.223 degrees, +/−5.224 degrees, +/−5.225 degrees, +/−5.226 degrees, +/−5.227 degrees, +/−5.228 degrees, +/−5.229 degrees, +/−5.230 degrees, +/−5.231 degrees, +/−5.232 degrees, +/−5.233 degrees, +/−5.234 degrees, +/−5.235 degrees, +/−5.236 degrees, +/−5.237 degrees, +/−5.238 degrees, +/−5.239 degrees, +/−5.240 degrees, +/−5.241 degrees, +/−5.242 degrees, +/−5.243 degrees, +/−5.244 degrees, +/−5.245 degrees, +/−5.246 degrees, +/−5.247 degrees, +/−5.248 degrees, +/−5.249 degrees, +/−5.250 degrees, +/−5.251 degrees, +/−5.252 degrees, +/−5.253 degrees, +/−5.254 degrees, +/−5.255 degrees, +/−5.256 degrees, +/−5.257 degrees, +/−5.258 degrees, +/−5.259 degrees, +/−5.260 degrees, +/−5.261 degrees, +/−5.262 degrees, +/−5.263 degrees, +/−5.264 degrees, +/−5.265 degrees, +/−5.266 degrees, +/−5.267 degrees, +/−5.268 degrees, +/−5.269 degrees, +/−5.270 degrees, +/−5.271 degrees, +/−5.272 degrees, +/−5.273 degrees, +/−5.274 degrees, +/−5.275 degrees, +/−5.276 degrees, +/−5.277 degrees, +/−5.278 degrees, +/−5.279 degrees, +/−5.280 degrees, +/−5.281 degrees, +/−5.282 degrees, +/−5.283 degrees, +/−5.284 degrees, +/−5.285 degrees, +/−5.286 degrees, +/−5.287 degrees, +/−5.288 degrees, +/−5.289 degrees, +/−5.290 degrees, +/−5.291 degrees, +/−5.292 degrees, +/−5.293 degrees, +/−5.294 degrees, +/−5.295 degrees, +/−5.296 degrees, +/−5.297 degrees, +/−5.298 degrees, +/−5.299 degrees, +/−5.300 degrees, degrees, +/−5.301 degrees, +/−5.302 degrees, +/−5.303 degrees, +/−5.304 degrees, +/−5.305 degrees, +/−5.306 degrees, +/−5.307 degrees, +/−5.308 degrees, +/−5.309 degrees, +/−5.310 degrees, +/−5.311 degrees, +/−5.312 degrees, +/−5.313 degrees, +/−5.314 degrees, +/−5.315 degrees, +/−5.316 degrees, +/−5.317 degrees, +/−5.318 degrees, +/−5.319 degrees, +/−5.320 degrees, +/−5.321 degrees, +/−5.322 degrees, +/−5.323 degrees, +/−5.324 degrees, +/−5.325 degrees, +/−5.326 degrees, +/−5.327 degrees, +/−5.328 degrees, +/−5.329 degrees, +/−5.330 degrees, +/−5.331 degrees, +/−5.332 degrees, +/−5.333 degrees, +/−5.334 degrees, +/−5.335 degrees, +/−5.336 degrees, +/−5.337 degrees, +/−5.338 degrees, +/−5.339 degrees, +/−5.340 degrees, +/−5.341 degrees, +/−5.342 degrees, +/−5.343 degrees, +/−5.344 degrees, +/−5.345 degrees, +/−5.346 degrees, +/−5.347 degrees, +/−5.348 degrees, +/−5.349 degrees, +/−5.350 degrees, +/−5.351 degrees, +/−5.352 degrees, +/−5.353 degrees, +/−5.354 degrees, +/−5.355 degrees, +/−5.356 degrees, +/−5.357 degrees, +/−5.358 degrees, +/−5.359 degrees, +/−5.360 degrees, +/−5.361 degrees, +/−5.362 degrees, +/−5.363 degrees, +/−5.364 degrees, +/−5.365 degrees, +/−5.366 degrees, +/−5.367 degrees, +/−5.368 degrees, +/−5.369 degrees, +/−5.370 degrees, +/−5.371 degrees, +/−5.372 degrees, +/−5.373 degrees, +/−5.374 degrees, +/−5.375 degrees, +/−5.376 degrees, +/−5.377 degrees, +/−5.378 degrees, +/−5.379 degrees, +/−5.380 degrees, +/−5.381 degrees, +/−5.382 degrees, +/−5.383 degrees, +/−5.384 degrees, +/−5.385 degrees, +/−5.386 degrees, +/−5.387 degrees, +/−5.388 degrees, +/−5.389 degrees, +/−5.390 degrees, +/−5.391 degrees, +/−5.392 degrees, +/−5.393 degrees, +/−5.394 degrees, +/−5.395 degrees, +/−5.396 degrees, +/−5.397 degrees, +/−5.398 degrees, +/−5.399 degrees, +/−5.400 degrees, degrees, +/−5.401 degrees, +/−5.402 degrees, +/−5.403 degrees, +/−5.404 degrees, +/−5.405 degrees, +/−5.406 degrees, +/−5.407 degrees, +/−5.408 degrees, +/−5.409 degrees, +/−5.410 degrees, +/−5.411 degrees, +/−5.412 degrees, +/−5.413 degrees, +/−5.414 degrees, +/−5.415 degrees, +/−5.416 degrees, +/−5.417 degrees, +/−5.418 degrees, +/−5.419 degrees, +/−5.420 degrees, +/−5.421 degrees, +/−5.422 degrees, +/−5.423 degrees, +/−5.424 degrees, +/−5.425 degrees, +/−5.426 degrees, +/−5.427 degrees, +/−5.428 degrees, +/−5.429 degrees, +/−5.430 degrees, +/−5.431 degrees, +/−5.432 degrees, +/−5.433 degrees, +/−5.434 degrees, +/−5.435 degrees, +/−5.436 degrees, +/−5.437 degrees, +/−5.438 degrees, +/−5.439 degrees, +/−5.440 degrees, +/−5.441 degrees, +/−5.442 degrees, +/−5.443 degrees, +/−5.444 degrees, +/−5.445 degrees, +/−5.446 degrees, +/−5.447 degrees, +/−5.448 degrees, +/−5.449 degrees, +/−5.450 degrees, +/−5.451 degrees, +/−5.452 degrees, +/−5.453 degrees, +/−5.454 degrees, +/−5.455 degrees, +/−5.456 degrees, +/−5.457 degrees, +/−5.458 degrees, +/−5.459 degrees, +/−5.460 degrees, +/−5.461 degrees, +/−5.462 degrees, +/−5.463 degrees, +/−5.464 degrees, +/−5.465 degrees, +/−5.466 degrees, +/−5.467 degrees, +/−5.468 degrees, +/−5.469 degrees, +/−5.470 degrees, +/−5.471 degrees, +/−5.472 degrees, +/−5.473 degrees, +/−5.474 degrees, +/−5.475 degrees, +/−5.476 degrees, +/−5.477 degrees, +/−5.478 degrees, +/−5.479 degrees, +/−5.480 degrees, +/−5.481 degrees, +/−5.482 degrees, +/−5.483 degrees, +/−5.484 degrees, +/−5.485 degrees, +/−5.486 degrees, +/−5.487 degrees, +/−5.488 degrees, +/−5.489 degrees, +/−5.490 degrees, +/−5.491 degrees, +/−5.492 degrees, +/−5.493 degrees, +/−5.494 degrees, +/−5.495 degrees, +/−5.496 degrees, +/−5.497 degrees, +/−5.498 degrees, +/−5.499 degrees, +/−5.500 degrees, degrees, +/−5.501 degrees, +/−5.502 degrees, +/−5.503 degrees, +/−5.504 degrees, +/−5.505 degrees, +/−5.506 degrees, +/−5.507 degrees, +/−5.508 degrees, +/−5.509 degrees, +/−5.510 degrees, +/−5.511 degrees, +/−5.512 degrees, +/−5.513 degrees, +/−5.514 degrees, +/−5.515 degrees, +/−5.516 degrees, +/−5.517 degrees, +/−5.518 degrees, +/−5.519 degrees, +/−5.520 degrees, +/−5.521 degrees, +/−5.522 degrees, +/−5.523 degrees, +/−5.524 degrees, +/−5.525 degrees, +/−5.526 degrees, +/−5.527 degrees, +/−5.528 degrees, +/−5.529 degrees, +/−5.530 degrees, +/−5.531 degrees, +/−5.532 degrees, +/−5.533 degrees, +/−5.534 degrees, +/−5.535 degrees, +/−5.536 degrees, +/−5.537 degrees, +/−5.538 degrees, +/−5.539 degrees, +/−5.540 degrees, +/−5.541 degrees, +/−5.542 degrees, +/−5.543 degrees, +/−5.544 degrees, +/−5.545 degrees, +/−5.546 degrees, +/−5.547 degrees, +/−5.548 degrees, +/−5.549 degrees, +/−5.550 degrees, +/−5.551 degrees, +/−5.552 degrees, +/−5.553 degrees, +/−5.554 degrees, +/−5.555 degrees, +/−5.556 degrees, +/−5.557 degrees, +/−5.558 degrees, +/−5.559 degrees, +/−5.560 degrees, +/−5.561 degrees, +/−5.562 degrees, +/−5.563 degrees, +/−5.564 degrees, +/−5.565 degrees, +/−5.566 degrees, +/−5.567 degrees, +/−5.568 degrees, +/−5.569 degrees, +/−5.570 degrees, +/−5.571 degrees, +/−5.572 degrees, +/−5.573 degrees, +/−5.574 degrees, +/−5.575 degrees, +/−5.576 degrees, +/−5.577 degrees, +/−5.578 degrees, +/−5.579 degrees, +/−5.580 degrees, +/−5.581 degrees, +/−5.582 degrees, +/−5.583 degrees, +/−5.584 degrees, +/−5.585 degrees, +/−5.586 degrees, +/−5.587 degrees, +/−5.588 degrees, +/−5.589 degrees, +/−5.590 degrees, +/−5.591 degrees, +/−5.592 degrees, +/−5.593 degrees, +/−5.594 degrees, +/−5.595 degrees, +/−5.596 degrees, +/−5.597 degrees, +/−5.598 degrees, +/−5.599 degrees, +/−5.600 degrees, degrees, +/−5.601 degrees, +/−5.602 degrees, +/−5.603 degrees, +/−5.604 degrees, +/−5.605 degrees, +/−5.606 degrees, +/−5.607 degrees, +/−5.608 degrees, +/−5.609 degrees, +/−5.610 degrees, +/−5.611 degrees, +/−5.612 degrees, +/−5.613 degrees, +/−5.614 degrees, +/−5.615 degrees, +/−5.616 degrees, +/−5.617 degrees, +/−5.618 degrees, +/−5.619 degrees, +/−5.620 degrees, +/−5.621 degrees, +/−5.622 degrees, +/−5.623 degrees, +/−5.624 degrees, +/−5.625 degrees, +/−5.626 degrees, +/−5.627 degrees, +/−5.628 degrees, +/−5.629 degrees, +/−5.630 degrees, +/−5.631 degrees, +/−5.632 degrees, +/−5.633 degrees, +/−5.634 degrees, +/−5.635 degrees, +/−5.636 degrees, +/−5.637 degrees, +/−5.638 degrees, +/−5.639 degrees, +/−5.640 degrees, +/−5.641 degrees, +/−5.642 degrees, +/−5.643 degrees, +/−5.644 degrees, +/−5.645 degrees, +/−5.646 degrees, +/−5.647 degrees, +/−5.648 degrees, +/−5.649 degrees, +/−5.650 degrees, +/−5.651 degrees, +/−5.652 degrees, +/−5.653 degrees, +/−5.654 degrees, +/−5.655 degrees, +/−5.656 degrees, +/−5.657 degrees, +/−5.658 degrees, +/−5.659 degrees, +/−5.660 degrees, +/−5.661 degrees, +/−5.662 degrees, +/−5.663 degrees, +/−5.664 degrees, +/−5.665 degrees, +/−5.666 degrees, +/−5.667 degrees, +/−5.668 degrees, +/−5.669 degrees, +/−5.670 degrees, +/−5.671 degrees, +/−5.672 degrees, +/−5.673 degrees, +/−5.674 degrees, +/−5.675 degrees, +/−5.676 degrees, +/−5.677 degrees, +/−5.678 degrees, +/−5.679 degrees, +/−5.680 degrees, +/−5.681 degrees, +/−5.682 degrees, +/−5.683 degrees, +/−5.684 degrees, +/−5.685 degrees, +/−5.686 degrees, +/−5.687 degrees, +/−5.688 degrees, +/−5.689 degrees, +/−5.690 degrees, +/−5.691 degrees, +/−5.692 degrees, +/−5.693 degrees, +/−5.694 degrees, +/−5.695 degrees, +/−5.696 degrees, +/−5.697 degrees, +/−5.698 degrees, +/−5.699 degrees, +/−5.700 degrees, degrees, +/−5.701 degrees, +/−5.702 degrees, +/−5.703 degrees, +/−5.704 degrees, +/−5.705 degrees, +/−5.706 degrees, +/−5.707 degrees, +/−5.708 degrees, +/−5.709 degrees, +/−5.710 degrees, +/−5.711 degrees, +/−5.712 degrees, +/−5.713 degrees, +/−5.714 degrees, +/−5.715 degrees, +/−5.716 degrees, +/−5.717 degrees, +/−5.718 degrees, +/−5.719 degrees, +/−5.720 degrees, +/−5.721 degrees, +/−5.722 degrees, +/−5.723 degrees, +/−5.724 degrees, +/−5.725 degrees, +/−5.726 degrees, +/−5.727 degrees, +/−5.728 degrees, +/−5.729 degrees, +/−5.730 degrees, +/−5.731 degrees, +/−5.732 degrees, +/−5.733 degrees, +/−5.734 degrees, +/−5.735 degrees, +/−5.736 degrees, +/−5.737 degrees, +/−5.738 degrees, +/−5.739 degrees, +/−5.740 degrees, +/−5.741 degrees, +/−5.742 degrees, +/−5.743 degrees, +/−5.744 degrees, +/−5.745 degrees, +/−5.746 degrees, +/−5.747 degrees, +/−5.748 degrees, +/−5.749 degrees, +/−5.750 degrees, +/−5.751 degrees, +/−5.752 degrees, +/−5.753 degrees, +/−5.754 degrees, +/−5.755 degrees, +/−5.756 degrees, +/−5.757 degrees, +/−5.758 degrees, +/−5.759 degrees, +/−5.760 degrees, +/−5.761 degrees, +/−5.762 degrees, +/−5.763 degrees, +/−5.764 degrees, +/−5.765 degrees, +/−5.766 degrees, +/−5.767 degrees, +/−5.768 degrees, +/−5.769 degrees, +/−5.770 degrees, +/−5.771 degrees, +/−5.772 degrees, +/−5.773 degrees, +/−5.774 degrees, +/−5.775 degrees, +/−5.776 degrees, +/−5.777 degrees, +/−5.778 degrees, +/−5.779 degrees, +/−5.780 degrees, +/−5.781 degrees, +/−5.782 degrees, +/−5.783 degrees, +/−5.784 degrees, +/−5.785 degrees, +/−5.786 degrees, +/−5.787 degrees, +/−5.788 degrees, +/−5.789 degrees, +/−5.790 degrees, +/−5.791 degrees, +/−5.792 degrees, +/−5.793 degrees, +/−5.794 degrees, +/−5.795 degrees, +/−5.796 degrees, +/−5.797 degrees, +/−5.798 degrees, +/−5.799 degrees, +/−5.800 degrees, degrees, +/−5.801 degrees, +/−5.802 degrees, +/−5.803 degrees, +/−5.804 degrees, +/−5.805 degrees, +/−5.806 degrees, +/−5.807 degrees, +/−5.808 degrees, +/−5.809 degrees, +/−5.810 degrees, +/−5.811 degrees, +/−5.812 degrees, +/−5.813 degrees, +/−5.814 degrees, +/−5.815 degrees, +/−5.816 degrees, +/−5.817 degrees, +/−5.818 degrees, +/−5.819 degrees, +/−5.820 degrees, +/−5.821 degrees, +/−5.822 degrees, +/−5.823 degrees, +/−5.824 degrees, +/−5.825 degrees, +/−5.826 degrees, +/−5.827 degrees, +/−5.828 degrees, +/−5.829 degrees, +/−5.830 degrees, +/−5.831 degrees, +/−5.832 degrees, +/−5.833 degrees, +/−5.834 degrees, +/−5.835 degrees, +/−5.836 degrees, +/−5.837 degrees, +/−5.838 degrees, +/−5.839 degrees, +/−5.840 degrees, +/−5.841 degrees, +/−5.842 degrees, +/−5.843 degrees, +/−5.844 degrees, +/−5.845 degrees, +/−5.846 degrees, +/−5.847 degrees, +/−5.848 degrees, +/−5.849 degrees, +/−5.850 degrees, +/−5.851 degrees, +/−5.852 degrees, +/−5.853 degrees, +/−5.854 degrees, +/−5.855 degrees, +/−5.856 degrees, +/−5.857 degrees, +/−5.858 degrees, +/−5.859 degrees, +/−5.860 degrees, +/−5.861 degrees, +/−5.862 degrees, +/−5.863 degrees, +/−5.864 degrees, +/−5.865 degrees, +/−5.866 degrees, +/−5.867 degrees, +/−5.868 degrees, +/−5.869 degrees, +/−5.870 degrees, +/−5.871 degrees, +/−5.872 degrees, +/−5.873 degrees, +/−5.874 degrees, +/−5.875 degrees, +/−5.876 degrees, +/−5.877 degrees, +/−5.878 degrees, +/−5.879 degrees, +/−5.880 degrees, +/−5.881 degrees, +/−5.882 degrees, +/−5.883 degrees, +/−5.884 degrees, +/−5.885 degrees, +/−5.886 degrees, +/−5.887 degrees, +/−5.888 degrees, +/−5.889 degrees, +/−5.890 degrees, +/−5.891 degrees, +/−5.892 degrees, +/−5.893 degrees, +/−5.894 degrees, +/−5.895 degrees, +/−5.896 degrees, +/−5.897 degrees, +/−5.898 degrees, +/−5.899 degrees, +/−5.900 degrees, degrees, +/−5.901 degrees, +/−5.902 degrees, +/−5.903 degrees, +/−5.904 degrees, +/−5.905 degrees, +/−5.906 degrees, +/−5.907 degrees, +/−5.908 degrees, +/−5.909 degrees, +/−5.910 degrees, +/−5.911 degrees, +/−5.912 degrees, +/−5.913 degrees, +/−5.914 degrees, +/−5.915 degrees, +/−5.916 degrees, +/−5.917 degrees, +/−5.918 degrees, +/−5.919 degrees, +/−5.920 degrees, +/−5.921 degrees, +/−5.922 degrees, +/−5.923 degrees, +/−5.924 degrees, +/−5.925 degrees, +/−5.926 degrees, +/−5.927 degrees, +/−5.928 degrees, +/−5.929 degrees, +/−5.930 degrees, +/−5.931 degrees, +/−5.932 degrees, +/−5.933 degrees, +/−5.934 degrees, +/−5.935 degrees, +/−5.936 degrees, +/−5.937 degrees, +/−5.938 degrees, +/−5.939 degrees, +/−5.940 degrees, +/−5.941 degrees, +/−5.942 degrees, +/−5.943 degrees, +/−5.944 degrees, +/−5.945 degrees, +/−5.946 degrees, +/−5.947 degrees, +/−5.948 degrees, +/−5.949 degrees, +/−5.950 degrees, +/−5.951 degrees, +/−5.952 degrees, +/−5.953 degrees, +/−5.954 degrees, +/−5.955 degrees, +/−5.956 degrees, +/−5.957 degrees, +/−5.958 degrees, +/−5.959 degrees, +/−5.960 degrees, +/−5.961 degrees, +/−5.962 degrees, +/−5.963 degrees, +/−5.964 degrees, +/−5.965 degrees, +/−5.966 degrees, +/−5.967 degrees, +/−5.968 degrees, +/−5.969 degrees, +/−5.970 degrees, +/−5.971 degrees, +/−5.972 degrees, +/−5.973 degrees, +/−5.974 degrees, +/−5.975 degrees, +/−5.976 degrees, +/−5.977 degrees, +/−5.978 degrees, +/−5.979 degrees, +/−5.980 degrees, +/−5.981 degrees, +/−5.982 degrees, +/−5.983 degrees, +/−5.984 degrees, +/−5.985 degrees, +/−5.986 degrees, +/−5.987 degrees, +/−5.988 degrees, +/−5.989 degrees, +/−5.990 degrees, +/−5.991 degrees, +/−5.992 degrees, +/−5.993 degrees, +/−5.994 degrees, +/−5.995 degrees, +/−5.996 degrees, +/−5.997 degrees, +/−5.998 degrees, +/−5.999 degrees, +/−6.000 degrees, +/−6.100, degrees, +/−6.101 degrees, +/−6.102 degrees, +/−6.103 degrees, +/−6.104 degrees, +/−6.105 degrees, +/−6.106 degrees, +/−6.107 degrees, +/−6.108 degrees, +/−6.109 degrees, +/−6.110 degrees, +/−6.111 degrees, +/−6.112 degrees, +/−6.113 degrees, +/−6.114 degrees, +/−6.115 degrees, +/−6.116 degrees, +/−6.117 degrees, +/−6.118 degrees, +/−6.119 degrees, +/−6.120 degrees, +/−6.121 degrees, +/−6.122 degrees, +/−6.123 degrees, +/−6.124 degrees, +/−6.125 degrees, +/−6.126 degrees, +/−6.127 degrees, +/−6.128 degrees, +/−6.129 degrees, +/−6.130 degrees, +/−6.131 degrees, +/−6.132 degrees, +/−6.133 degrees, +/−6.134 degrees, +/−6.135 degrees, +/−6.136 degrees, +/−6.137 degrees, +/−6.138 degrees, +/−6.139 degrees, +/−6.140 degrees, +/−6.141 degrees, +/−6.142 degrees, +/−6.143 degrees, +/−6.144 degrees, +/−6.145 degrees, +/−6.146 degrees, +/−6.147 degrees, +/−6.148 degrees, +/−6.149 degrees, +/−6.150 degrees, +/−6.151 degrees, +/−6.152 degrees, +/−6.153 degrees, +/−6.154 degrees, +/−6.155 degrees, +/−6.156 degrees, +/−6.157 degrees, +/−6.158 degrees, +/−6.159 degrees, +/−6.160 degrees, +/−6.161 degrees, +/−6.162 degrees, +/−6.163 degrees, +/−6.164 degrees, +/−6.165 degrees, +/−6.166 degrees, +/−6.167 degrees, +/−6.168 degrees, +/−6.169 degrees, +/−6.170 degrees, +/−6.171 degrees, +/−6.172 degrees, +/−6.173 degrees, +/−6.174 degrees, +/−6.175 degrees, +/−6.176 degrees, +/−6.177 degrees, +/−6.178 degrees, +/−6.179 degrees, +/−6.180 degrees, +/−6.181 degrees, +/−6.182 degrees, +/−6.183 degrees, +/−6.184 degrees, +/−6.185 degrees, +/−6.186 degrees, +/−6.187 degrees, +/−6.188 degrees, +/−6.189 degrees, +/−6.190 degrees, +/−6.191 degrees, +/−6.192 degrees, +/−6.193 degrees, +/−6.194 degrees, +/−6.195 degrees, +/−6.196 degrees, +/−6.197 degrees, +/−6.198 degrees, +/−6.199 degrees, +/−6.200 degrees, degrees, +/−6.201 degrees, +/−6.202 degrees, +/−6.203 degrees, +/−6.204 degrees, +/−6.205 degrees, +/−6.206 degrees, +/−6.207 degrees, +/−6.208 degrees, +/−6.209 degrees, +/−6.210 degrees, +/−6.211 degrees, +/−6.212 degrees, +/−6.213 degrees, +/−6.214 degrees, +/−6.215 degrees, +/−6.216 degrees, +/−6.217 degrees, +/−6.218 degrees, +/−6.219 degrees, +/−6.220 degrees, +/−6.221 degrees, +/−6.222 degrees, +/−6.223 degrees, +/−6.224 degrees, +/−6.225 degrees, +/−6.226 degrees, +/−6.227 degrees, +/−6.228 degrees, +/−6.229 degrees, +/−6.230 degrees, +/−6.231 degrees, +/−6.232 degrees, +/−6.233 degrees, +/−6.234 degrees, +/−6.235 degrees, +/−6.236 degrees, +/−6.237 degrees, +/−6.238 degrees, +/−6.239 degrees, +/−6.240 degrees, +/−6.241 degrees, +/−6.242 degrees, +/−6.243 degrees, +/−6.244 degrees, +/−6.245 degrees, +/−6.246 degrees, +/−6.247 degrees, +/−6.248 degrees, +/−6.249 degrees, +/−6.250 degrees, +/−6.251 degrees, +/−6.252 degrees, +/−6.253 degrees, +/−6.254 degrees, +/−6.255 degrees, +/−6.256 degrees, +/−6.257 degrees, +/−6.258 degrees, +/−6.259 degrees, +/−6.260 degrees, +/−6.261 degrees, +/−6.262 degrees, +/−6.263 degrees, +/−6.264 degrees, +/−6.265 degrees, +/−6.266 degrees, +/−6.267 degrees, +/−6.268 degrees, +/−6.269 degrees, +/−6.270 degrees, +/−6.271 degrees, +/−6.272 degrees, +/−6.273 degrees, +/−6.274 degrees, +/−6.275 degrees, +/−6.276 degrees, +/−6.277 degrees, +/−6.278 degrees, +/−6.279 degrees, +/−6.280 degrees, +/−6.281 degrees, +/−6.282 degrees, +/−6.283 degrees, +/−6.284 degrees, +/−6.285 degrees, +/−6.286 degrees, +/−6.287 degrees, +/−6.288 degrees, +/−6.289 degrees, +/−6.290 degrees, +/−6.291 degrees, +/−6.292 degrees, +/−6.293 degrees, +/−6.294 degrees, +/−6.295 degrees, +/−6.296 degrees, +/−6.297 degrees, +/−6.298 degrees, +/−6.299 degrees, +/−6.300 degrees, degrees, +/−6.301 degrees, +/−6.302 degrees, +/−6.303 degrees, +/−6.304 degrees, +/−6.305 degrees, +/−6.306 degrees, +/−6.307 degrees, +/−6.308 degrees, +/−6.309 degrees, +/−6.310 degrees, +/−6.311 degrees, +/−6.312 degrees, +/−6.313 degrees, +/−6.314 degrees, +/−6.315 degrees, +/−6.316 degrees, +/−6.317 degrees, +/−6.318 degrees, +/−6.319 degrees, +/−6.320 degrees, +/−6.321 degrees, +/−6.322 degrees, +/−6.323 degrees, +/−6.324 degrees, +/−6.325 degrees, +/−6.326 degrees, +/−6.327 degrees, +/−6.328 degrees, +/−6.329 degrees, +/−6.330 degrees, +/−6.331 degrees, +/−6.332 degrees, +/−6.333 degrees, +/−6.334 degrees, +/−6.335 degrees, +/−6.336 degrees, +/−6.337 degrees, +/−6.338 degrees, +/−6.339 degrees, +/−6.340 degrees, +/−6.341 degrees, +/−6.342 degrees, +/−6.343 degrees, +/−6.344 degrees, +/−6.345 degrees, +/−6.346 degrees, +/−6.347 degrees, +/−6.348 degrees, +/−6.349 degrees, +/−6.350 degrees, +/−6.351 degrees, +/−6.352 degrees, +/−6.353 degrees, +/−6.354 degrees, +/−6.355 degrees, +/−6.356 degrees, +/−6.357 degrees, +/−6.358 degrees, +/−6.359 degrees, +/−6.360 degrees, +/−6.361 degrees, +/−6.362 degrees, +/−6.363 degrees, +/−6.364 degrees, +/−6.365 degrees, +/−6.366 degrees, +/−6.367 degrees, +/−6.368 degrees, +/−6.369 degrees, +/−6.370 degrees, +/−6.371 degrees, +/−6.372 degrees, +/−6.373 degrees, +/−6.374 degrees, +/−6.375 degrees, +/−6.376 degrees, +/−6.377 degrees, +/−6.378 degrees, +/−6.379 degrees, +/−6.380 degrees, +/−6.381 degrees, +/−6.382 degrees, +/−6.383 degrees, +/−6.384 degrees, +/−6.385 degrees, +/−6.386 degrees, +/−6.387 degrees, +/−6.388 degrees, +/−6.389 degrees, +/−6.390 degrees, +/−6.391 degrees, +/−6.392 degrees, +/−6.393 degrees, +/−6.394 degrees, +/−6.395 degrees, +/−6.396 degrees, +/−6.397 degrees, +/−6.398 degrees, +/−6.399 degrees, +/−6.400 degrees, degrees, +/−6.401 degrees, +/−6.402 degrees, +/−6.403 degrees, +/−6.404 degrees, +/−6.405 degrees, +/−6.406 degrees, +/−6.407 degrees, +/−6.408 degrees, +/−6.409 degrees, +/−6.410 degrees, +/−6.411 degrees, +/−6.412 degrees, +/−6.413 degrees, +/−6.414 degrees, +/−6.415 degrees, +/−6.416 degrees, +/−6.417 degrees, +/−6.418 degrees, +/−6.419 degrees, +/−6.420 degrees, +/−6.421 degrees, +/−6.422 degrees, +/−6.423 degrees, +/−6.424 degrees, +/−6.425 degrees, +/−6.426 degrees, +/−6.427 degrees, +/−6.428 degrees, +/−6.429 degrees, +/−6.430 degrees, +/−6.431 degrees, +/−6.432 degrees, +/−6.433 degrees, +/−6.434 degrees, +/−6.435 degrees, +/−6.436 degrees, +/−6.437 degrees, +/−6.438 degrees, +/−6.439 degrees, +/−6.440 degrees, +/−6.441 degrees, +/−6.442 degrees, +/−6.443 degrees, +/−6.444 degrees, +/−6.445 degrees, +/−6.446 degrees, +/−6.447 degrees, +/−6.448 degrees, +/−6.449 degrees, +/−6.450 degrees, +/−6.451 degrees, +/−6.452 degrees, +/−6.453 degrees, +/−6.454 degrees, +/−6.455 degrees, +/−6.456 degrees, +/−6.457 degrees, +/−6.458 degrees, +/−6.459 degrees, +/−6.460 degrees, +/−6.461 degrees, +/−6.462 degrees, +/−6.463 degrees, +/−6.464 degrees, +/−6.465 degrees, +/−6.466 degrees, +/−6.467 degrees, +/−6.468 degrees, +/−6.469 degrees, +/−6.470 degrees, +/−6.471 degrees, +/−6.472 degrees, +/−6.473 degrees, +/−6.474 degrees, +/−6.475 degrees, +/−6.476 degrees, +/−6.477 degrees, +/−6.478 degrees, +/−6.479 degrees, +/−6.480 degrees, +/−6.481 degrees, +/−6.482 degrees, +/−6.483 degrees, +/−6.484 degrees, +/−6.485 degrees, +/−6.486 degrees, +/−6.487 degrees, +/−6.488 degrees, +/−6.489 degrees, +/−6.490 degrees, +/−6.491 degrees, +/−6.492 degrees, +/−6.493 degrees, +/−6.494 degrees, +/−6.495 degrees, +/−6.496 degrees, +/−6.497 degrees, +/−6.498 degrees, +/−6.499 degrees, +/−6.500 degrees, degrees, +/−6.501 degrees, +/−6.502 degrees, +/−6.503 degrees, +/−6.504 degrees, +/−6.505 degrees, +/−6.506 degrees, +/−6.507 degrees, +/−6.508 degrees, +/−6.509 degrees, +/−6.510 degrees, +/−6.511 degrees, +/−6.512 degrees, +/−6.513 degrees, +/−6.514 degrees, +/−6.515 degrees, +/−6.516 degrees, +/−6.517 degrees, +/−6.518 degrees, +/−6.519 degrees, +/−6.520 degrees, +/−6.521 degrees, +/−6.522 degrees, +/−6.523 degrees, +/−6.524 degrees, +/−6.525 degrees, +/−6.526 degrees, +/−6.527 degrees, +/−6.528 degrees, +/−6.529 degrees, +/−6.530 degrees, +/−6.531 degrees, +/−6.532 degrees, +/−6.533 degrees, +/−6.534 degrees, +/−6.535 degrees, +/−6.536 degrees, +/−6.537 degrees, +/−6.538 degrees, +/−6.539 degrees, +/−6.540 degrees, +/−6.541 degrees, +/−6.542 degrees, +/−6.543 degrees, +/−6.544 degrees, +/−6.545 degrees, +/−6.546 degrees, +/−6.547 degrees, +/−6.548 degrees, +/−6.549 degrees, +/−6.550 degrees, +/−6.551 degrees, +/−6.552 degrees, +/−6.553 degrees, +/−6.554 degrees, +/−6.555 degrees, +/−6.556 degrees, +/−6.557 degrees, +/−6.558 degrees, +/−6.559 degrees, +/−6.560 degrees, +/−6.561 degrees, +/−6.562 degrees, +/−6.563 degrees, +/−6.564 degrees, +/−6.565 degrees, +/−6.566 degrees, +/−6.567 degrees, +/−6.568 degrees, +/−6.569 degrees, +/−6.570 degrees, +/−6.571 degrees, +/−6.572 degrees, +/−6.573 degrees, +/−6.574 degrees, +/−6.575 degrees, +/−6.576 degrees, +/−6.577 degrees, +/−6.578 degrees, +/−6.579 degrees, +/−6.580 degrees, +/−6.581 degrees, +/−6.582 degrees, +/−6.583 degrees, +/−6.584 degrees, +/−6.585 degrees, +/−6.586 degrees, +/−6.587 degrees, +/−6.588 degrees, +/−6.589 degrees, +/−6.590 degrees, +/−6.591 degrees, +/−6.592 degrees, +/−6.593 degrees, +/−6.594 degrees, +/−6.595 degrees, +/−6.596 degrees, +/−6.597 degrees, +/−6.598 degrees, +/−6.599 degrees, +/−6.600 degrees, degrees, +/−6.601 degrees, +/−6.602 degrees, +/−6.603 degrees, +/−6.604 degrees, +/−6.605 degrees, +/−6.606 degrees, +/−6.607 degrees, +/−6.608 degrees, +/−6.609 degrees, +/−6.610 degrees, +/−6.611 degrees, +/−6.612 degrees, +/−6.613 degrees, +/−6.614 degrees, +/−6.615 degrees, +/−6.616 degrees, +/−6.617 degrees, +/−6.618 degrees, +/−6.619 degrees, +/−6.620 degrees, +/−6.621 degrees, +/−6.622 degrees, +/−6.623 degrees, +/−6.624 degrees, +/−6.625 degrees, +/−6.626 degrees, +/−6.627 degrees, +/−6.628 degrees, +/−6.629 degrees, +/−6.630 degrees, +/−6.631 degrees, +/−6.632 degrees, +/−6.633 degrees, +/−6.634 degrees, +/−6.635 degrees, +/−6.636 degrees, +/−6.637 degrees, +/−6.638 degrees, +/−6.639 degrees, +/−6.640 degrees, +/−6.641 degrees, +/−6.642 degrees, +/−6.643 degrees, +/−6.644 degrees, +/−6.645 degrees, +/−6.646 degrees, +/−6.647 degrees, +/−6.648 degrees, +/−6.649 degrees, +/−6.650 degrees, +/−6.651 degrees, +/−6.652 degrees, +/−6.653 degrees, +/−6.654 degrees, +/−6.655 degrees, +/−6.656 degrees, +/−6.657 degrees, +/−6.658 degrees, +/−6.659 degrees, +/−6.660 degrees, +/−6.661 degrees, +/−6.662 degrees, +/−6.663 degrees, +/−6.664 degrees, +/−6.665 degrees, +/−6.666 degrees, +/−6.667 degrees, +/−6.668 degrees, +/−6.669 degrees, +/−6.670 degrees, +/−6.671 degrees, +/−6.672 degrees, +/−6.673 degrees, +/−6.674 degrees, +/−6.675 degrees, +/−6.676 degrees, +/−6.677 degrees, +/−6.678 degrees, +/−6.679 degrees, +/−6.680 degrees, +/−6.681 degrees, +/−6.682 degrees, +/−6.683 degrees, +/−6.684 degrees, +/−6.685 degrees, +/−6.686 degrees, +/−6.687 degrees, +/−6.688 degrees, +/−6.689 degrees, +/−6.690 degrees, +/−6.691 degrees, +/−6.692 degrees, +/−6.693 degrees, +/−6.694 degrees, +/−6.695 degrees, +/−6.696 degrees, +/−6.697 degrees, +/−6.698 degrees, +/−6.699 degrees, +/−6.700 degrees, degrees, +/−6.701 degrees, +/−6.702 degrees, +/−6.703 degrees, +/−6.704 degrees, +/−6.705 degrees, +/−6.706 degrees, +/−6.707 degrees, +/−6.708 degrees, +/−6.709 degrees, +/−6.710 degrees, +/−6.711 degrees, +/−6.712 degrees, +/−6.713 degrees, +/−6.714 degrees, +/−6.715 degrees, +/−6.716 degrees, +/−6.717 degrees, +/−6.718 degrees, +/−6.719 degrees, +/−6.720 degrees, +/−6.721 degrees, +/−6.722 degrees, +/−6.723 degrees, +/−6.724 degrees, +/−6.725 degrees, +/−6.726 degrees, +/−6.727 degrees, +/−6.728 degrees, +/−6.729 degrees, +/−6.730 degrees, +/−6.731 degrees, +/−6.732 degrees, +/−6.733 degrees, +/−6.734 degrees, +/−6.735 degrees, +/−6.736 degrees, +/−6.737 degrees, +/−6.738 degrees, +/−6.739 degrees, +/−6.740 degrees, +/−6.741 degrees, +/−6.742 degrees, +/−6.743 degrees, +/−6.744 degrees, +/−6.745 degrees, +/−6.746 degrees, +/−6.747 degrees, +/−6.748 degrees, +/−6.749 degrees, +/−6.750 degrees, +/−6.751 degrees, +/−6.752 degrees, +/−6.753 degrees, +/−6.754 degrees, +/−6.755 degrees, +/−6.756 degrees, +/−6.757 degrees, +/−6.758 degrees, +/−6.759 degrees, +/−6.760 degrees, +/−6.761 degrees, +/−6.762 degrees, +/−6.763 degrees, +/−6.764 degrees, +/−6.765 degrees, +/−6.766 degrees, +/−6.767 degrees, +/−6.768 degrees, +/−6.769 degrees, +/−6.770 degrees, +/−6.771 degrees, +/−6.772 degrees, +/−6.773 degrees, +/−6.774 degrees, +/−6.775 degrees, +/−6.776 degrees, +/−6.777 degrees, +/−6.778 degrees, +/−6.779 degrees, +/−6.780 degrees, +/−6.781 degrees, +/−6.782 degrees, +/−6.783 degrees, +/−6.784 degrees, +/−6.785 degrees, +/−6.786 degrees, +/−6.787 degrees, +/−6.788 degrees, +/−6.789 degrees, +/−6.790 degrees, +/−6.791 degrees, +/−6.792 degrees, +/−6.793 degrees, +/−6.794 degrees, +/−6.795 degrees, +/−6.796 degrees, +/−6.797 degrees, +/−6.798 degrees, +/−6.799 degrees, +/−6.800 degrees, degrees, +/−6.801 degrees, +/−6.802 degrees, +/−6.803 degrees, +/−6.804 degrees, +/−6.805 degrees, +/−6.806 degrees, +/−6.807 degrees, +/−6.808 degrees, +/−6.809 degrees, +/−6.810 degrees, +/−6.811 degrees, +/−6.812 degrees, +/−6.813 degrees, +/−6.814 degrees, +/−6.815 degrees, +/−6.816 degrees, +/−6.817 degrees, +/−6.818 degrees, +/−6.819 degrees, +/−6.820 degrees, +/−6.821 degrees, +/−6.822 degrees, +/−6.823 degrees, +/−6.824 degrees, +/−6.825 degrees, +/−6.826 degrees, +/−6.827 degrees, +/−6.828 degrees, +/−6.829 degrees, +/−6.830 degrees, +/−6.831 degrees, +/−6.832 degrees, +/−6.833 degrees, +/−6.834 degrees, +/−6.835 degrees, +/−6.836 degrees, +/−6.837 degrees, +/−6.838 degrees, +/−6.839 degrees, +/−6.840 degrees, +/−6.841 degrees, +/−6.842 degrees, +/−6.843 degrees, +/−6.844 degrees, +/−6.845 degrees, +/−6.846 degrees, +/−6.847 degrees, +/−6.848 degrees, +/−6.849 degrees, +/−6.850 degrees, +/−6.851 degrees, +/−6.852 degrees, +/−6.853 degrees, +/−6.854 degrees, +/−6.855 degrees, +/−6.856 degrees, +/−6.857 degrees, +/−6.858 degrees, +/−6.859 degrees, +/−6.860 degrees, +/−6.861 degrees, +/−6.862 degrees, +/−6.863 degrees, +/−6.864 degrees, +/−6.865 degrees, +/−6.866 degrees, +/−6.867 degrees, +/−6.868 degrees, +/−6.869 degrees, +/−6.870 degrees, +/−6.871 degrees, +/−6.872 degrees, +/−6.873 degrees, +/−6.874 degrees, +/−6.875 degrees, +/−6.876 degrees, +/−6.877 degrees, +/−6.878 degrees, +/−6.879 degrees, +/−6.880 degrees, +/−6.881 degrees, +/−6.882 degrees, +/−6.883 degrees, +/−6.884 degrees, +/−6.885 degrees, +/−6.886 degrees, +/−6.887 degrees, +/−6.888 degrees, +/−6.889 degrees, +/−6.890 degrees, +/−6.891 degrees, +/−6.892 degrees, +/−6.893 degrees, +/−6.894 degrees, +/−6.895 degrees, +/−6.896 degrees, +/−6.897 degrees, +/−6.898 degrees, +/−6.899 degrees, +/−6.900 degrees, degrees, +/−6.901 degrees, +/−6.902 degrees, +/−6.903 degrees, +/−6.904 degrees, +/−6.905 degrees, +/−6.906 degrees, +/−6.907 degrees, +/−6.908 degrees, +/−6.909 degrees, +/−6.910 degrees, +/−6.911 degrees, +/−6.912 degrees, +/−6.913 degrees, +/−6.914 degrees, +/−6.915 degrees, +/−6.916 degrees, +/−6.917 degrees, +/−6.918 degrees, +/−6.919 degrees, +/−6.920 degrees, +/−6.921 degrees, +/−6.922 degrees, +/−6.923 degrees, +/−6.924 degrees, +/−6.925 degrees, +/−6.926 degrees, +/−6.927 degrees, +/−6.928 degrees, +/−6.929 degrees, +/−6.930 degrees, +/−6.931 degrees, +/−6.932 degrees, +/−6.933 degrees, +/−6.934 degrees, +/−6.935 degrees, +/−6.936 degrees, +/−6.937 degrees, +/−6.938 degrees, +/−6.939 degrees, +/−6.940 degrees, +/−6.941 degrees, +/−6.942 degrees, +/−6.943 degrees, +/−6.944 degrees, +/−6.945 degrees, +/−6.946 degrees, +/−6.947 degrees, +/−6.948 degrees, +/−6.949 degrees, +/−6.950 degrees, +/−6.951 degrees, +/−6.952 degrees, +/−6.953 degrees, +/−6.954 degrees, +/−6.955 degrees, +/−6.956 degrees, +/−6.957 degrees, +/−6.958 degrees, +/−6.959 degrees, +/−6.960 degrees, +/−6.961 degrees, +/−6.962 degrees, +/−6.963 degrees, +/−6.964 degrees, +/−6.965 degrees, +/−6.966 degrees, +/−6.967 degrees, +/−6.968 degrees, +/−6.969 degrees, +/−6.970 degrees, +/−6.971 degrees, +/−6.972 degrees, +/−6.973 degrees, +/−6.974 degrees, +/−6.975 degrees, +/−6.976 degrees, +/−6.977 degrees, +/−6.978 degrees, +/−6.979 degrees, +/−6.980 degrees, +/−6.981 degrees, +/−6.982 degrees, +/−6.983 degrees, +/−6.984 degrees, +/−6.985 degrees, +/−6.986 degrees, +/−6.987 degrees, +/−6.988 degrees, +/−6.989 degrees, +/−6.990 degrees, +/−6.991 degrees, +/−6.992 degrees, +/−6.993 degrees, +/−6.994 degrees, +/−6.995 degrees, +/−6.996 degrees, +/−6.997 degrees, +/−6.998 degrees, +/−6.999 degrees, +/−7.000 degrees, +/−7.100, degrees, +/−7.101 degrees, +/−7.102 degrees, +/−7.103 degrees, +/−7.104 degrees, +/−7.105 degrees, +/−7.106 degrees, +/−7.107 degrees, +/−7.108 degrees, +/−7.109 degrees, +/−7.110 degrees, +/−7.111 degrees, +/−7.112 degrees, +/−7.113 degrees, +/−7.114 degrees, +/−7.115 degrees, +/−7.116 degrees, +/−7.117 degrees, +/−7.118 degrees, +/−7.119 degrees, +/−7.120 degrees, +/−7.121 degrees, +/−7.122 degrees, +/−7.123 degrees, +/−7.124 degrees, +/−7.125 degrees, +/−7.126 degrees, +/−7.127 degrees, +/−7.128 degrees, +/−7.129 degrees, +/−7.130 degrees, +/−7.131 degrees, +/−7.132 degrees, +/−7.133 degrees, +/−7.134 degrees, +/−7.135 degrees, +/−7.136 degrees, +/−7.137 degrees, +/−7.138 degrees, +/−7.139 degrees, +/−7.140 degrees, +/−7.141 degrees, +/−7.142 degrees, +/−7.143 degrees, +/−7.144 degrees, +/−7.145 degrees, +/−7.146 degrees, +/−7.147 degrees, +/−7.148 degrees, +/−7.149 degrees, +/−7.150 degrees, +/−7.151 degrees, +/−7.152 degrees, +/−7.153 degrees, +/−7.154 degrees, +/−7.155 degrees, +/−7.156 degrees, +/−7.157 degrees, +/−7.158 degrees, +/−7.159 degrees, +/−7.160 degrees, +/−7.161 degrees, +/−7.162 degrees, +/−7.163 degrees, +/−7.164 degrees, +/−7.165 degrees, +/−7.166 degrees, +/−7.167 degrees, +/−7.168 degrees, +/−7.169 degrees, +/−7.170 degrees, +/−7.171 degrees, +/−7.172 degrees, +/−7.173 degrees, +/−7.174 degrees, +/−7.175 degrees, +/−7.176 degrees, +/−7.177 degrees, +/−7.178 degrees, +/−7.179 degrees, +/−7.180 degrees, +/−7.181 degrees, +/−7.182 degrees, +/−7.183 degrees, +/−7.184 degrees, +/−7.185 degrees, +/−7.186 degrees, +/−7.187 degrees, +/−7.188 degrees, +/−7.189 degrees, +/−7.190 degrees, +/−7.191 degrees, +/−7.192 degrees, +/−7.193 degrees, +/−7.194 degrees, +/−7.195 degrees, +/−7.196 degrees, +/−7.197 degrees, +/−7.198 degrees, +/−7.199 degrees, +/−7.200 degrees, degrees, +/−7.201 degrees, +/−7.202 degrees, +/−7.203 degrees, +/−7.204 degrees, +/−7.205 degrees, +/−7.206 degrees, +/−7.207 degrees, +/−7.208 degrees, +/−7.209 degrees, +/−7.210 degrees, +/−7.211 degrees, +/−7.212 degrees, +/−7.213 degrees, +/−7.214 degrees, +/−7.215 degrees, +/−7.216 degrees, +/−7.217 degrees, +/−7.218 degrees, +/−7.219 degrees, +/−7.220 degrees, +/−7.221 degrees, +/−7.222 degrees, +/−7.223 degrees, +/−7.224 degrees, +/−7.225 degrees, +/−7.226 degrees, +/−7.227 degrees, +/−7.228 degrees, +/−7.229 degrees, +/−7.230 degrees, +/−7.231 degrees, +/−7.232 degrees, +/−7.233 degrees, +/−7.234 degrees, +/−7.235 degrees, +/−7.236 degrees, +/−7.237 degrees, +/−7.238 degrees, +/−7.239 degrees, +/−7.240 degrees, +/−7.241 degrees, +/−7.242 degrees, +/−7.243 degrees, +/−7.244 degrees, +/−7.245 degrees, +/−7.246 degrees, +/−7.247 degrees, +/−7.248 degrees, +/−7.249 degrees, +/−7.250 degrees, +/−7.251 degrees, +/−7.252 degrees, +/−7.253 degrees, +/−7.254 degrees, +/−7.255 degrees, +/−7.256 degrees, +/−7.257 degrees, +/−7.258 degrees, +/−7.259 degrees, +/−7.260 degrees, +/−7.261 degrees, +/−7.262 degrees, +/−7.263 degrees, +/−7.264 degrees, +/−7.265 degrees, +/−7.266 degrees, +/−7.267 degrees, +/−7.268 degrees, +/−7.269 degrees, +/−7.270 degrees, +/−7.271 degrees, +/−7.272 degrees, +/−7.273 degrees, +/−7.274 degrees, +/−7.275 degrees, +/−7.276 degrees, +/−7.277 degrees, +/−7.278 degrees, +/−7.279 degrees, +/−7.280 degrees, +/−7.281 degrees, +/−7.282 degrees, +/−7.283 degrees, +/−7.284 degrees, +/−7.285 degrees, +/−7.286 degrees, +/−7.287 degrees, +/−7.288 degrees, +/−7.289 degrees, +/−7.290 degrees, +/−7.291 degrees, +/−7.292 degrees, +/−7.293 degrees, +/−7.294 degrees, +/−7.295 degrees, +/−7.296 degrees, +/−7.297 degrees, +/−7.298 degrees, +/−7.299 degrees, +/−7.300 degrees, degrees, +/−7.301 degrees, +/−7.302 degrees, +/−7.303 degrees, +/−7.304 degrees, +/−7.305 degrees, +/−7.306 degrees, +/−7.307 degrees, +/−7.308 degrees, +/−7.309 degrees, +/−7.310 degrees, +/−7.311 degrees, +/−7.312 degrees, +/−7.313 degrees, +/−7.314 degrees, +/−7.315 degrees, +/−7.316 degrees, +/−7.317 degrees, +/−7.318 degrees, +/−7.319 degrees, +/−7.320 degrees, +/−7.321 degrees, +/−7.322 degrees, +/−7.323 degrees, +/−7.324 degrees, +/−7.325 degrees, +/−7.326 degrees, +/−7.327 degrees, +/−7.328 degrees, +/−7.329 degrees, +/−7.330 degrees, +/−7.331 degrees, +/−7.332 degrees, +/−7.333 degrees, +/−7.334 degrees, +/−7.335 degrees, +/−7.336 degrees, +/−7.337 degrees, +/−7.338 degrees, +/−7.339 degrees, +/−7.340 degrees, +/−7.341 degrees, +/−7.342 degrees, +/−7.343 degrees, +/−7.344 degrees, +/−7.345 degrees, +/−7.346 degrees, +/−7.347 degrees, +/−7.348 degrees, +/−7.349 degrees, +/−7.350 degrees, +/−7.351 degrees, +/−7.352 degrees, +/−7.353 degrees, +/−7.354 degrees, +/−7.355 degrees, +/−7.356 degrees, +/−7.357 degrees, +/−7.358 degrees, +/−7.359 degrees, +/−7.360 degrees, +/−7.361 degrees, +/−7.362 degrees, +/−7.363 degrees, +/−7.364 degrees, +/−7.365 degrees, +/−7.366 degrees, +/−7.367 degrees, +/−7.368 degrees, +/−7.369 degrees, +/−7.370 degrees, +/−7.371 degrees, +/−7.372 degrees, +/−7.373 degrees, +/−7.374 degrees, +/−7.375 degrees, +/−7.376 degrees, +/−7.377 degrees, +/−7.378 degrees, +/−7.379 degrees, +/−7.380 degrees, +/−7.381 degrees, +/−7.382 degrees, +/−7.383 degrees, +/−7.384 degrees, +/−7.385 degrees, +/−7.386 degrees, +/−7.387 degrees, +/−7.388 degrees, +/−7.389 degrees, +/−7.390 degrees, +/−7.391 degrees, +/−7.392 degrees, +/−7.393 degrees, +/−7.394 degrees, +/−7.395 degrees, +/−7.396 degrees, +/−7.397 degrees, +/−7.398 degrees, +/−7.399 degrees, +/−7.400 degrees, degrees, +/−7.401 degrees, +/−7.402 degrees, +/−7.403 degrees, +/−7.404 degrees, +/−7.405 degrees, +/−7.406 degrees, +/−7.407 degrees, +/−7.408 degrees, +/−7.409 degrees, +/−7.410 degrees, +/−7.411 degrees, +/−7.412 degrees, +/−7.413 degrees, +/−7.414 degrees, +/−7.415 degrees, +/−7.416 degrees, +/−7.417 degrees, +/−7.418 degrees, +/−7.419 degrees, +/−7.420 degrees, +/−7.421 degrees, +/−7.422 degrees, +/−7.423 degrees, +/−7.424 degrees, +/−7.425 degrees, +/−7.426 degrees, +/−7.427 degrees, +/−7.428 degrees, +/−7.429 degrees, +/−7.430 degrees, +/−7.431 degrees, +/−7.432 degrees, +/−7.433 degrees, +/−7.434 degrees, +/−7.435 degrees, +/−7.436 degrees, +/−7.437 degrees, +/−7.438 degrees, +/−7.439 degrees, +/−7.440 degrees, +/−7.441 degrees, +/−7.442 degrees, +/−7.443 degrees, +/−7.444 degrees, +/−7.445 degrees, +/−7.446 degrees, +/−7.447 degrees, +/−7.448 degrees, +/−7.449 degrees, +/−7.450 degrees, +/−7.451 degrees, +/−7.452 degrees, +/−7.453 degrees, +/−7.454 degrees, +/−7.455 degrees, +/−7.456 degrees, +/−7.457 degrees, +/−7.458 degrees, +/−7.459 degrees, +/−7.460 degrees, +/−7.461 degrees, +/−7.462 degrees, +/−7.463 degrees, +/−7.464 degrees, +/−7.465 degrees, +/−7.466 degrees, +/−7.467 degrees, +/−7.468 degrees, +/−7.469 degrees, +/−7.470 degrees, +/−7.471 degrees, +/−7.472 degrees, +/−7.473 degrees, +/−7.474 degrees, +/−7.475 degrees, +/−7.476 degrees, +/−7.477 degrees, +/−7.478 degrees, +/−7.479 degrees, +/−7.480 degrees, +/−7.481 degrees, +/−7.482 degrees, +/−7.483 degrees, +/−7.484 degrees, +/−7.485 degrees, +/−7.486 degrees, +/−7.487 degrees, +/−7.488 degrees, +/−7.489 degrees, +/−7.490 degrees, +/−7.491 degrees, +/−7.492 degrees, +/−7.493 degrees, +/−7.494 degrees, +/−7.495 degrees, +/−7.496 degrees, +/−7.497 degrees, +/−7.498 degrees, +/−7.499 degrees, +/−7.500 degrees, degrees, +/−7.501 degrees, +/−7.502 degrees, +/−7.503 degrees, +/−7.504 degrees, +/−7.505 degrees, +/−7.506 degrees, +/−7.507 degrees, +/−7.508 degrees, +/−7.509 degrees, +/−7.510 degrees, +/−7.511 degrees, +/−7.512 degrees, +/−7.513 degrees, +/−7.514 degrees, +/−7.515 degrees, +/−7.516 degrees, +/−7.517 degrees, +/−7.518 degrees, +/−7.519 degrees, +/−7.520 degrees, +/−7.521 degrees, +/−7.522 degrees, +/−7.523 degrees, +/−7.524 degrees, +/−7.525 degrees, +/−7.526 degrees, +/−7.527 degrees, +/−7.528 degrees, +/−7.529 degrees, +/−7.530 degrees, +/−7.531 degrees, +/−7.532 degrees, +/−7.533 degrees, +/−7.534 degrees, +/−7.535 degrees, +/−7.536 degrees, +/−7.537 degrees, +/−7.538 degrees, +/−7.539 degrees, +/−7.540 degrees, +/−7.541 degrees, +/−7.542 degrees, +/−7.543 degrees, +/−7.544 degrees, +/−7.545 degrees, +/−7.546 degrees, +/−7.547 degrees, +/−7.548 degrees, +/−7.549 degrees, +/−7.550 degrees, +/−7.551 degrees, +/−7.552 degrees, +/−7.553 degrees, +/−7.554 degrees, +/−7.555 degrees, +/−7.556 degrees, +/−7.557 degrees, +/−7.558 degrees, +/−7.559 degrees, +/−7.560 degrees, +/−7.561 degrees, +/−7.562 degrees, +/−7.563 degrees, +/−7.564 degrees, +/−7.565 degrees, +/−7.566 degrees, +/−7.567 degrees, +/−7.568 degrees, +/−7.569 degrees, +/−7.570 degrees, +/−7.571 degrees, +/−7.572 degrees, +/−7.573 degrees, +/−7.574 degrees, +/−7.575 degrees, +/−7.576 degrees, +/−7.577 degrees, +/−7.578 degrees, +/−7.579 degrees, +/−7.580 degrees, +/−7.581 degrees, +/−7.582 degrees, +/−7.583 degrees, +/−7.584 degrees, +/−7.585 degrees, +/−7.586 degrees, +/−7.587 degrees, +/−7.588 degrees, +/−7.589 degrees, +/−7.590 degrees, +/−7.591 degrees, +/−7.592 degrees, +/−7.593 degrees, +/−7.594 degrees, +/−7.595 degrees, +/−7.596 degrees, +/−7.597 degrees, +/−7.598 degrees, +/−7.599 degrees, +/−7.600 degrees, degrees, +/−7.601 degrees, +/−7.602 degrees, +/−7.603 degrees, +/−7.604 degrees, +/−7.605 degrees, +/−7.606 degrees, +/−7.607 degrees, +/−7.608 degrees, +/−7.609 degrees, +/−7.610 degrees, +/−7.611 degrees, +/−7.612 degrees, +/−7.613 degrees, +/−7.614 degrees, +/−7.615 degrees, +/−7.616 degrees, +/−7.617 degrees, +/−7.618 degrees, +/−7.619 degrees, +/−7.620 degrees, +/−7.621 degrees, +/−7.622 degrees, +/−7.623 degrees, +/−7.624 degrees, +/−7.625 degrees, +/−7.626 degrees, +/−7.627 degrees, +/−7.628 degrees, +/−7.629 degrees, +/−7.630 degrees, +/−7.631 degrees, +/−7.632 degrees, +/−7.633 degrees, +/−7.634 degrees, +/−7.635 degrees, +/−7.636 degrees, +/−7.637 degrees, +/−7.638 degrees, +/−7.639 degrees, +/−7.640 degrees, +/−7.641 degrees, +/−7.642 degrees, +/−7.643 degrees, +/−7.644 degrees, +/−7.645 degrees, +/−7.646 degrees, +/−7.647 degrees, +/−7.648 degrees, +/−7.649 degrees, +/−7.650 degrees, +/−7.651 degrees, +/−7.652 degrees, +/−7.653 degrees, +/−7.654 degrees, +/−7.655 degrees, +/−7.656 degrees, +/−7.657 degrees, +/−7.658 degrees, +/−7.659 degrees, +/−7.660 degrees, +/−7.661 degrees, +/−7.662 degrees, +/−7.663 degrees, +/−7.664 degrees, +/−7.665 degrees, +/−7.666 degrees, +/−7.667 degrees, +/−7.668 degrees, +/−7.669 degrees, +/−7.670 degrees, +/−7.671 degrees, +/−7.672 degrees, +/−7.673 degrees, +/−7.674 degrees, +/−7.675 degrees, +/−7.676 degrees, +/−7.677 degrees, +/−7.678 degrees, +/−7.679 degrees, +/−7.680 degrees, +/−7.681 degrees, +/−7.682 degrees, +/−7.683 degrees, +/−7.684 degrees, +/−7.685 degrees, +/−7.686 degrees, +/−7.687 degrees, +/−7.688 degrees, +/−7.689 degrees, +/−7.690 degrees, +/−7.691 degrees, +/−7.692 degrees, +/−7.693 degrees, +/−7.694 degrees, +/−7.695 degrees, +/−7.696 degrees, +/−7.697 degrees, +/−7.698 degrees, +/−7.699 degrees, +/−7.700 degrees, degrees, +/−7.701 degrees, +/−7.702 degrees, +/−7.703 degrees, +/−7.704 degrees, +/−7.705 degrees, +/−7.706 degrees, +/−7.707 degrees, +/−7.708 degrees, +/−7.709 degrees, +/−7.710 degrees, +/−7.711 degrees, +/−7.712 degrees, +/−7.713 degrees, +/−7.714 degrees, +/−7.715 degrees, +/−7.716 degrees, +/−7.717 degrees, +/−7.718 degrees, +/−7.719 degrees, +/−7.720 degrees, +/−7.721 degrees, +/−7.722 degrees, +/−7.723 degrees, +/−7.724 degrees, +/−7.725 degrees, +/−7.726 degrees, +/−7.727 degrees, +/−7.728 degrees, +/−7.729 degrees, +/−7.730 degrees, +/−7.731 degrees, +/−7.732 degrees, +/−7.733 degrees, +/−7.734 degrees, +/−7.735 degrees, +/−7.736 degrees, +/−7.737 degrees, +/−7.738 degrees, +/−7.739 degrees, +/−7.740 degrees, +/−7.741 degrees, +/−7.742 degrees, +/−7.743 degrees, +/−7.744 degrees, +/−7.745 degrees, +/−7.746 degrees, +/−7.747 degrees, +/−7.748 degrees, +/−7.749 degrees, +/−7.750 degrees, +/−7.751 degrees, +/−7.752 degrees, +/−7.753 degrees, +/−7.754 degrees, +/−7.755 degrees, +/−7.756 degrees, +/−7.757 degrees, +/−7.758 degrees, +/−7.759 degrees, +/−7.760 degrees, +/−7.761 degrees, +/−7.762 degrees, +/−7.763 degrees, +/−7.764 degrees, +/−7.765 degrees, +/−7.766 degrees, +/−7.767 degrees, +/−7.768 degrees, +/−7.769 degrees, +/−7.770 degrees, +/−7.771 degrees, +/−7.772 degrees, +/−7.773 degrees, +/−7.774 degrees, +/−7.775 degrees, +/−7.776 degrees, +/−7.777 degrees, +/−7.778 degrees, +/−7.779 degrees, +/−7.780 degrees, +/−7.781 degrees, +/−7.782 degrees, +/−7.783 degrees, +/−7.784 degrees, +/−7.785 degrees, +/−7.786 degrees, +/−7.787 degrees, +/−7.788 degrees, +/−7.789 degrees, +/−7.790 degrees, +/−7.791 degrees, +/−7.792 degrees, +/−7.793 degrees, +/−7.794 degrees, +/−7.795 degrees, +/−7.796 degrees, +/−7.797 degrees, +/−7.798 degrees, +/−7.799 degrees, +/−7.800 degrees, degrees, +/−7.801 degrees, +/−7.802 degrees, +/−7.803 degrees, +/−7.804 degrees, +/−7.805 degrees, +/−7.806 degrees, +/−7.807 degrees, +/−7.808 degrees, +/−7.809 degrees, +/−7.810 degrees, +/−7.811 degrees, +/−7.812 degrees, +/−7.813 degrees, +/−7.814 degrees, +/−7.815 degrees, +/−7.816 degrees, +/−7.817 degrees, +/−7.818 degrees, +/−7.819 degrees, +/−7.820 degrees, +/−7.821 degrees, +/−7.822 degrees, +/−7.823 degrees, +/−7.824 degrees, +/−7.825 degrees, +/−7.826 degrees, +/−7.827 degrees, +/−7.828 degrees, +/−7.829 degrees, +/−7.830 degrees, +/−7.831 degrees, +/−7.832 degrees, +/−7.833 degrees, +/−7.834 degrees, +/−7.835 degrees, +/−7.836 degrees, +/−7.837 degrees, +/−7.838 degrees, +/−7.839 degrees, +/−7.840 degrees, +/−7.841 degrees, +/−7.842 degrees, +/−7.843 degrees, +/−7.844 degrees, +/−7.845 degrees, +/−7.846 degrees, +/−7.847 degrees, +/−7.848 degrees, +/−7.849 degrees, +/−7.850 degrees, +/−7.851 degrees, +/−7.852 degrees, +/−7.853 degrees, +/−7.854 degrees, +/−7.855 degrees, +/−7.856 degrees, +/−7.857 degrees, +/−7.858 degrees, +/−7.859 degrees, +/−7.860 degrees, +/−7.861 degrees, +/−7.862 degrees, +/−7.863 degrees, +/−7.864 degrees, +/−7.865 degrees, +/−7.866 degrees, +/−7.867 degrees, +/−7.868 degrees, +/−7.869 degrees, +/−7.870 degrees, +/−7.871 degrees, +/−7.872 degrees, +/−7.873 degrees, +/−7.874 degrees, +/−7.875 degrees, +/−7.876 degrees, +/−7.877 degrees, +/−7.878 degrees, +/−7.879 degrees, +/−7.880 degrees, +/−7.881 degrees, +/−7.882 degrees, +/−7.883 degrees, +/−7.884 degrees, +/−7.885 degrees, +/−7.886 degrees, +/−7.887 degrees, +/−7.888 degrees, +/−7.889 degrees, +/−7.890 degrees, +/−7.891 degrees, +/−7.892 degrees, +/−7.893 degrees, +/−7.894 degrees, +/−7.895 degrees, +/−7.896 degrees, +/−7.897 degrees, +/−7.898 degrees, +/−7.899 degrees, +/−7.900 degrees, degrees, +/−7.901 degrees, +/−7.902 degrees, +/−7.903 degrees, +/−7.904 degrees, +/−7.905 degrees, +/−7.906 degrees, +/−7.907 degrees, +/−7.908 degrees, +/−7.909 degrees, +/−7.910 degrees, +/−7.911 degrees, +/−7.912 degrees, +/−7.913 degrees, +/−7.914 degrees, +/−7.915 degrees, +/−7.916 degrees, +/−7.917 degrees, +/−7.918 degrees, +/−7.919 degrees, +/−7.920 degrees, +/−7.921 degrees, +/−7.922 degrees, +/−7.923 degrees, +/−7.924 degrees, +/−7.925 degrees, +/−7.926 degrees, +/−7.927 degrees, +/−7.928 degrees, +/−7.929 degrees, +/−7.930 degrees, +/−7.931 degrees, +/−7.932 degrees, +/−7.933 degrees, +/−7.934 degrees, +/−7.935 degrees, +/−7.936 degrees, +/−7.937 degrees, +/−7.938 degrees, +/−7.939 degrees, +/−7.940 degrees, +/−7.941 degrees, +/−7.942 degrees, +/−7.943 degrees, +/−7.944 degrees, +/−7.945 degrees, +/−7.946 degrees, +/−7.947 degrees, +/−7.948 degrees, +/−7.949 degrees, +/−7.950 degrees, +/−7.951 degrees, +/−7.952 degrees, +/−7.953 degrees, +/−7.954 degrees, +/−7.955 degrees, +/−7.956 degrees, +/−7.957 degrees, +/−7.958 degrees, +/−7.959 degrees, +/−7.960 degrees, +/−7.961 degrees, +/−7.962 degrees, +/−7.963 degrees, +/−7.964 degrees, +/−7.965 degrees, +/−7.966 degrees, +/−7.967 degrees, +/−7.968 degrees, +/−7.969 degrees, +/−7.970 degrees, +/−7.971 degrees, +/−7.972 degrees, +/−7.973 degrees, +/−7.974 degrees, +/−7.975 degrees, +/−7.976 degrees, +/−7.977 degrees, +/−7.978 degrees, +/−7.979 degrees, +/−7.980 degrees, +/−7.981 degrees, +/−7.982 degrees, +/−7.983 degrees, +/−7.984 degrees, +/−7.985 degrees, +/−7.986 degrees, +/−7.987 degrees, +/−7.988 degrees, +/−7.989 degrees, +/−7.990 degrees, +/−7.991 degrees, +/−7.992 degrees, +/−7.993 degrees, +/−7.994 degrees, +/−7.995 degrees, +/−7.996 degrees, +/−7.997 degrees, +/−7.998 degrees, +/−7.999 degrees, +/−8.000 degrees, +/−8.100, degrees, +/−8.101 degrees, +/−8.102 degrees, +/−8.103 degrees, +/−8.104 degrees, +/−8.105 degrees, +/−8.106 degrees, +/−8.107 degrees, +/−8.108 degrees, +/−8.109 degrees, +/−8.110 degrees, +/−8.111 degrees, +/−8.112 degrees, +/−8.113 degrees, +/−8.114 degrees, +/−8.115 degrees, +/−8.116 degrees, +/−8.117 degrees, +/−8.118 degrees, +/−8.119 degrees, +/−8.120 degrees, +/−8.121 degrees, +/−8.122 degrees, +/−8.123 degrees, +/−8.124 degrees, +/−8.125 degrees, +/−8.126 degrees, +/−8.127 degrees, +/−8.128 degrees, +/−8.129 degrees, +/−8.130 degrees, +/−8.131 degrees, +/−8.132 degrees, +/−8.133 degrees, +/−8.134 degrees, +/−8.135 degrees, +/−8.136 degrees, +/−8.137 degrees, +/−8.138 degrees, +/−8.139 degrees, +/−8.140 degrees, +/−8.141 degrees, +/−8.142 degrees, +/−8.143 degrees, +/−8.144 degrees, +/−8.145 degrees, +/−8.146 degrees, +/−8.147 degrees, +/−8.148 degrees, +/−8.149 degrees, +/−8.150 degrees, +/−8.151 degrees, +/−8.152 degrees, +/−8.153 degrees, +/−8.154 degrees, +/−8.155 degrees, +/−8.156 degrees, +/−8.157 degrees, +/−8.158 degrees, +/−8.159 degrees, +/−8.160 degrees, +/−8.161 degrees, +/−8.162 degrees, +/−8.163 degrees, +/−8.164 degrees, +/−8.165 degrees, +/−8.166 degrees, +/−8.167 degrees, +/−8.168 degrees, +/−8.169 degrees, +/−8.170 degrees, +/−8.171 degrees, +/−8.172 degrees, +/−8.173 degrees, +/−8.174 degrees, +/−8.175 degrees, +/−8.176 degrees, +/−8.177 degrees, +/−8.178 degrees, +/−8.179 degrees, +/−8.180 degrees, +/−8.181 degrees, +/−8.182 degrees, +/−8.183 degrees, +/−8.184 degrees, +/−8.185 degrees, +/−8.186 degrees, +/−8.187 degrees, +/−8.188 degrees, +/−8.189 degrees, +/−8.190 degrees, +/−8.191 degrees, +/−8.192 degrees, +/−8.193 degrees, +/−8.194 degrees, +/−8.195 degrees, +/−8.196 degrees, +/−8.197 degrees, +/−8.198 degrees, +/−8.199 degrees, +/−8.200 degrees, degrees, +/−8.201 degrees, +/−8.202 degrees, +/−8.203 degrees, +/−8.204 degrees, +/−8.205 degrees, +/−8.206 degrees, +/−8.207 degrees, +/−8.208 degrees, +/−8.209 degrees, +/−8.210 degrees, +/−8.211 degrees, +/−8.212 degrees, +/−8.213 degrees, +/−8.214 degrees, +/−8.215 degrees, +/−8.216 degrees, +/−8.217 degrees, +/−8.218 degrees, +/−8.219 degrees, +/−8.220 degrees, +/−8.221 degrees, +/−8.222 degrees, +/−8.223 degrees, +/−8.224 degrees, +/−8.225 degrees, +/−8.226 degrees, +/−8.227 degrees, +/−8.228 degrees, +/−8.229 degrees, +/−8.230 degrees, +/−8.231 degrees, +/−8.232 degrees, +/−8.233 degrees, +/−8.234 degrees, +/−8.235 degrees, +/−8.236 degrees, +/−8.237 degrees, +/−8.238 degrees, +/−8.239 degrees, +/−8.240 degrees, +/−8.241 degrees, +/−8.242 degrees, +/−8.243 degrees, +/−8.244 degrees, +/−8.245 degrees, +/−8.246 degrees, +/−8.247 degrees, +/−8.248 degrees, +/−8.249 degrees, +/−8.250 degrees, +/−8.251 degrees, +/−8.252 degrees, +/−8.253 degrees, +/−8.254 degrees, +/−8.255 degrees, +/−8.256 degrees, +/−8.257 degrees, +/−8.258 degrees, +/−8.259 degrees, +/−8.260 degrees, +/−8.261 degrees, +/−8.262 degrees, +/−8.263 degrees, +/−8.264 degrees, +/−8.265 degrees, +/−8.266 degrees, +/−8.267 degrees, +/−8.268 degrees, +/−8.269 degrees, +/−8.270 degrees, +/−8.271 degrees, +/−8.272 degrees, +/−8.273 degrees, +/−8.274 degrees, +/−8.275 degrees, +/−8.276 degrees, +/−8.277 degrees, +/−8.278 degrees, +/−8.279 degrees, +/−8.280 degrees, +/−8.281 degrees, +/−8.282 degrees, +/−8.283 degrees, +/−8.284 degrees, +/−8.285 degrees, +/−8.286 degrees, +/−8.287 degrees, +/−8.288 degrees, +/−8.289 degrees, +/−8.290 degrees, +/−8.291 degrees, +/−8.292 degrees, +/−8.293 degrees, +/−8.294 degrees, +/−8.295 degrees, +/−8.296 degrees, +/−8.297 degrees, +/−8.298 degrees, +/−8.299 degrees, +/−8.300 degrees, degrees, +/−8.301 degrees, +/−8.302 degrees, +/−8.303 degrees, +/−8.304 degrees, +/−8.305 degrees, +/−8.306 degrees, +/−8.307 degrees, +/−8.308 degrees, +/−8.309 degrees, +/−8.310 degrees, +/−8.311 degrees, +/−8.312 degrees, +/−8.313 degrees, +/−8.314 degrees, +/−8.315 degrees, +/−8.316 degrees, +/−8.317 degrees, +/−8.318 degrees, +/−8.319 degrees, +/−8.320 degrees, +/−8.321 degrees, +/−8.322 degrees, +/−8.323 degrees, +/−8.324 degrees, +/−8.325 degrees, +/−8.326 degrees, +/−8.327 degrees, +/−8.328 degrees, +/−8.329 degrees, +/−8.330 degrees, +/−8.331 degrees, +/−8.332 degrees, +/−8.333 degrees, +/−8.334 degrees, +/−8.335 degrees, +/−8.336 degrees, +/−8.337 degrees, +/−8.338 degrees, +/−8.339 degrees, +/−8.340 degrees, +/−8.341 degrees, +/−8.342 degrees, +/−8.343 degrees, +/−8.344 degrees, +/−8.345 degrees, +/−8.346 degrees, +/−8.347 degrees, +/−8.348 degrees, +/−8.349 degrees, +/−8.350 degrees, +/−8.351 degrees, +/−8.352 degrees, +/−8.353 degrees, +/−8.354 degrees, +/−8.355 degrees, +/−8.356 degrees, +/−8.357 degrees, +/−8.358 degrees, +/−8.359 degrees, +/−8.360 degrees, +/−8.361 degrees, +/−8.362 degrees, +/−8.363 degrees, +/−8.364 degrees, +/−8.365 degrees, +/−8.366 degrees, +/−8.367 degrees, +/−8.368 degrees, +/−8.369 degrees, +/−8.370 degrees, +/−8.371 degrees, +/−8.372 degrees, +/−8.373 degrees, +/−8.374 degrees, +/−8.375 degrees, +/−8.376 degrees, +/−8.377 degrees, +/−8.378 degrees, +/−8.379 degrees, +/−8.380 degrees, +/−8.381 degrees, +/−8.382 degrees, +/−8.383 degrees, +/−8.384 degrees, +/−8.385 degrees, +/−8.386 degrees, +/−8.387 degrees, +/−8.388 degrees, +/−8.389 degrees, +/−8.390 degrees, +/−8.391 degrees, +/−8.392 degrees, +/−8.393 degrees, +/−8.394 degrees, +/−8.395 degrees, +/−8.396 degrees, +/−8.397 degrees, +/−8.398 degrees, +/−8.399 degrees, +/−8.400 degrees, degrees, +/−8.401 degrees, +/−8.402 degrees, +/−8.403 degrees, +/−8.404 degrees, +/−8.405 degrees, +/−8.406 degrees, +/−8.407 degrees, +/−8.408 degrees, +/−8.409 degrees, +/−8.410 degrees, +/−8.411 degrees, +/−8.412 degrees, +/−8.413 degrees, +/−8.414 degrees, +/−8.415 degrees, +/−8.416 degrees, +/−8.417 degrees, +/−8.418 degrees, +/−8.419 degrees, +/−8.420 degrees, +/−8.421 degrees, +/−8.422 degrees, +/−8.423 degrees, +/−8.424 degrees, +/−8.425 degrees, +/−8.426 degrees, +/−8.427 degrees, +/−8.428 degrees, +/−8.429 degrees, +/−8.430 degrees, +/−8.431 degrees, +/−8.432 degrees, +/−8.433 degrees, +/−8.434 degrees, +/−8.435 degrees, +/−8.436 degrees, +/−8.437 degrees, +/−8.438 degrees, +/−8.439 degrees, +/−8.440 degrees, +/−8.441 degrees, +/−8.442 degrees, +/−8.443 degrees, +/−8.444 degrees, +/−8.445 degrees, +/−8.446 degrees, +/−8.447 degrees, +/−8.448 degrees, +/−8.449 degrees, +/−8.450 degrees, +/−8.451 degrees, +/−8.452 degrees, +/−8.453 degrees, +/−8.454 degrees, +/−8.455 degrees, +/−8.456 degrees, +/−8.457 degrees, +/−8.458 degrees, +/−8.459 degrees, +/−8.460 degrees, +/−8.461 degrees, +/−8.462 degrees, +/−8.463 degrees, +/−8.464 degrees, +/−8.465 degrees, +/−8.466 degrees, +/−8.467 degrees, +/−8.468 degrees, +/−8.469 degrees, +/−8.470 degrees, +/−8.471 degrees, +/−8.472 degrees, +/−8.473 degrees, +/−8.474 degrees, +/−8.475 degrees, +/−8.476 degrees, +/−8.477 degrees, +/−8.478 degrees, +/−8.479 degrees, +/−8.480 degrees, +/−8.481 degrees, +/−8.482 degrees, +/−8.483 degrees, +/−8.484 degrees, +/−8.485 degrees, +/−8.486 degrees, +/−8.487 degrees, +/−8.488 degrees, +/−8.489 degrees, +/−8.490 degrees, +/−8.491 degrees, +/−8.492 degrees, +/−8.493 degrees, +/−8.494 degrees, +/−8.495 degrees, +/−8.496 degrees, +/−8.497 degrees, +/−8.498 degrees, +/−8.499 degrees, +/−8.500 degrees, degrees, +/−8.501 degrees, +/−8.502 degrees, +/−8.503 degrees, +/−8.504 degrees, +/−8.505 degrees, +/−8.506 degrees, +/−8.507 degrees, +/−8.508 degrees, +/−8.509 degrees, +/−8.510 degrees, +/−8.511 degrees, +/−8.512 degrees, +/−8.513 degrees, +/−8.514 degrees, +/−8.515 degrees, +/−8.516 degrees, +/−8.517 degrees, +/−8.518 degrees, +/−8.519 degrees, +/−8.520 degrees, +/−8.521 degrees, +/−8.522 degrees, +/−8.523 degrees, +/−8.524 degrees, +/−8.525 degrees, +/−8.526 degrees, +/−8.527 degrees, +/−8.528 degrees, +/−8.529 degrees, +/−8.530 degrees, +/−8.531 degrees, +/−8.532 degrees, +/−8.533 degrees, +/−8.534 degrees, +/−8.535 degrees, +/−8.536 degrees, +/−8.537 degrees, +/−8.538 degrees, +/−8.539 degrees, +/−8.540 degrees, +/−8.541 degrees, +/−8.542 degrees, +/−8.543 degrees, +/−8.544 degrees, +/−8.545 degrees, +/−8.546 degrees, +/−8.547 degrees, +/−8.548 degrees, +/−8.549 degrees, +/−8.550 degrees, +/−8.551 degrees, +/−8.552 degrees, +/−8.553 degrees, +/−8.554 degrees, +/−8.555 degrees, +/−8.556 degrees, +/−8.557 degrees, +/−8.558 degrees, +/−8.559 degrees, +/−8.560 degrees, +/−8.561 degrees, +/−8.562 degrees, +/−8.563 degrees, +/−8.564 degrees, +/−8.565 degrees, +/−8.566 degrees, +/−8.567 degrees, +/−8.568 degrees, +/−8.569 degrees, +/−8.570 degrees, +/−8.571 degrees, +/−8.572 degrees, +/−8.573 degrees, +/−8.574 degrees, +/−8.575 degrees, +/−8.576 degrees, +/−8.577 degrees, +/−8.578 degrees, +/−8.579 degrees, +/−8.580 degrees, +/−8.581 degrees, +/−8.582 degrees, +/−8.583 degrees, +/−8.584 degrees, +/−8.585 degrees, +/−8.586 degrees, +/−8.587 degrees, +/−8.588 degrees, +/−8.589 degrees, +/−8.590 degrees, +/−8.591 degrees, +/−8.592 degrees, +/−8.593 degrees, +/−8.594 degrees, +/−8.595 degrees, +/−8.596 degrees, +/−8.597 degrees, +/−8.598 degrees, +/−8.599 degrees, +/−8.600 degrees, degrees, +/−8.601 degrees, +/−8.602 degrees, +/−8.603 degrees, +/−8.604 degrees, +/−8.605 degrees, +/−8.606 degrees, +/−8.607 degrees, +/−8.608 degrees, +/−8.609 degrees, +/−8.610 degrees, +/−8.611 degrees, +/−8.612 degrees, +/−8.613 degrees, +/−8.614 degrees, +/−8.615 degrees, +/−8.616 degrees, +/−8.617 degrees, +/−8.618 degrees, +/−8.619 degrees, +/−8.620 degrees, +/−8.621 degrees, +/−8.622 degrees, +/−8.623 degrees, +/−8.624 degrees, +/−8.625 degrees, +/−8.626 degrees, +/−8.627 degrees, +/−8.628 degrees, +/−8.629 degrees, +/−8.630 degrees, +/−8.631 degrees, +/−8.632 degrees, +/−8.633 degrees, +/−8.634 degrees, +/−8.635 degrees, +/−8.636 degrees, +/−8.637 degrees, +/−8.638 degrees, +/−8.639 degrees, +/−8.640 degrees, +/−8.641 degrees, +/−8.642 degrees, +/−8.643 degrees, +/−8.644 degrees, +/−8.645 degrees, +/−8.646 degrees, +/−8.647 degrees, +/−8.648 degrees, +/−8.649 degrees, +/−8.650 degrees, +/−8.651 degrees, +/−8.652 degrees, +/−8.653 degrees, +/−8.654 degrees, +/−8.655 degrees, +/−8.656 degrees, +/−8.657 degrees, +/−8.658 degrees, +/−8.659 degrees, +/−8.660 degrees, +/−8.661 degrees, +/−8.662 degrees, +/−8.663 degrees, +/−8.664 degrees, +/−8.665 degrees, +/−8.666 degrees, +/−8.667 degrees, +/−8.668 degrees, +/−8.669 degrees, +/−8.670 degrees, +/−8.671 degrees, +/−8.672 degrees, +/−8.673 degrees, +/−8.674 degrees, +/−8.675 degrees, +/−8.676 degrees, +/−8.677 degrees, +/−8.678 degrees, +/−8.679 degrees, +/−8.680 degrees, +/−8.681 degrees, +/−8.682 degrees, +/−8.683 degrees, +/−8.684 degrees, +/−8.685 degrees, +/−8.686 degrees, +/−8.687 degrees, +/−8.688 degrees, +/−8.689 degrees, +/−8.690 degrees, +/−8.691 degrees, +/−8.692 degrees, +/−8.693 degrees, +/−8.694 degrees, +/−8.695 degrees, +/−8.696 degrees, +/−8.697 degrees, +/−8.698 degrees, +/−8.699 degrees, +/−8.700 degrees, degrees, +/−8.701 degrees, +/−8.702 degrees, +/−8.703 degrees, +/−8.704 degrees, +/−8.705 degrees, +/−8.706 degrees, +/−8.707 degrees, +/−8.708 degrees, +/−8.709 degrees, +/−8.710 degrees, +/−8.711 degrees, +/−8.712 degrees, +/−8.713 degrees, +/−8.714 degrees, +/−8.715 degrees, +/−8.716 degrees, +/−8.717 degrees, +/−8.718 degrees, +/−8.719 degrees, +/−8.720 degrees, +/−8.721 degrees, +/−8.722 degrees, +/−8.723 degrees, +/−8.724 degrees, +/−8.725 degrees, +/−8.726 degrees, +/−8.727 degrees, +/−8.728 degrees, +/−8.729 degrees, +/−8.730 degrees, +/−8.731 degrees, +/−8.732 degrees, +/−8.733 degrees, +/−8.734 degrees, +/−8.735 degrees, +/−8.736 degrees, +/−8.737 degrees, +/−8.738 degrees, +/−8.739 degrees, +/−8.740 degrees, +/−8.741 degrees, +/−8.742 degrees, +/−8.743 degrees, +/−8.744 degrees, +/−8.745 degrees, +/−8.746 degrees, +/−8.747 degrees, +/−8.748 degrees, +/−8.749 degrees, +/−8.750 degrees, +/−8.751 degrees, +/−8.752 degrees, +/−8.753 degrees, +/−8.754 degrees, +/−8.755 degrees, +/−8.756 degrees, +/−8.757 degrees, +/−8.758 degrees, +/−8.759 degrees, +/−8.760 degrees, +/−8.761 degrees, +/−8.762 degrees, +/−8.763 degrees, +/−8.764 degrees, +/−8.765 degrees, +/−8.766 degrees, +/−8.767 degrees, +/−8.768 degrees, +/−8.769 degrees, +/−8.770 degrees, +/−8.771 degrees, +/−8.772 degrees, +/−8.773 degrees, +/−8.774 degrees, +/−8.775 degrees, +/−8.776 degrees, +/−8.777 degrees, +/−8.778 degrees, +/−8.779 degrees, +/−8.780 degrees, +/−8.781 degrees, +/−8.782 degrees, +/−8.783 degrees, +/−8.784 degrees, +/−8.785 degrees, +/−8.786 degrees, +/−8.787 degrees, +/−8.788 degrees, +/−8.789 degrees, +/−8.790 degrees, +/−8.791 degrees, +/−8.792 degrees, +/−8.793 degrees, +/−8.794 degrees, +/−8.795 degrees, +/−8.796 degrees, +/−8.797 degrees, +/−8.798 degrees, +/−8.799 degrees, +/−8.800 degrees, degrees, +/−8.801 degrees, +/−8.802 degrees, +/−8.803 degrees, +/−8.804 degrees, +/−8.805 degrees, +/−8.806 degrees, +/−8.807 degrees, +/−8.808 degrees, +/−8.809 degrees, +/−8.810 degrees, +/−8.811 degrees, +/−8.812 degrees, +/−8.813 degrees, +/−8.814 degrees, +/−8.815 degrees, +/−8.816 degrees, +/−8.817 degrees, +/−8.818 degrees, +/−8.819 degrees, +/−8.820 degrees, +/−8.821 degrees, +/−8.822 degrees, +/−8.823 degrees, +/−8.824 degrees, +/−8.825 degrees, +/−8.826 degrees, +/−8.827 degrees, +/−8.828 degrees, +/−8.829 degrees, +/−8.830 degrees, +/−8.831 degrees, +/−8.832 degrees, +/−8.833 degrees, +/−8.834 degrees, +/−8.835 degrees, +/−8.836 degrees, +/−8.837 degrees, +/−8.838 degrees, +/−8.839 degrees, +/−8.840 degrees, +/−8.841 degrees, +/−8.842 degrees, +/−8.843 degrees, +/−8.844 degrees, +/−8.845 degrees, +/−8.846 degrees, +/−8.847 degrees, +/−8.848 degrees, +/−8.849 degrees, +/−8.850 degrees, +/−8.851 degrees, +/−8.852 degrees, +/−8.853 degrees, +/−8.854 degrees, +/−8.855 degrees, +/−8.856 degrees, +/−8.857 degrees, +/−8.858 degrees, +/−8.859 degrees, +/−8.860 degrees, +/−8.861 degrees, +/−8.862 degrees, +/−8.863 degrees, +/−8.864 degrees, +/−8.865 degrees, +/−8.866 degrees, +/−8.867 degrees, +/−8.868 degrees, +/−8.869 degrees, +/−8.870 degrees, +/−8.871 degrees, +/−8.872 degrees, +/−8.873 degrees, +/−8.874 degrees, +/−8.875 degrees, +/−8.876 degrees, +/−8.877 degrees, +/−8.878 degrees, +/−8.879 degrees, +/−8.880 degrees, +/−8.881 degrees, +/−8.882 degrees, +/−8.883 degrees, +/−8.884 degrees, +/−8.885 degrees, +/−8.886 degrees, +/−8.887 degrees, +/−8.888 degrees, +/−8.889 degrees, +/−8.890 degrees, +/−8.891 degrees, +/−8.892 degrees, +/−8.893 degrees, +/−8.894 degrees, +/−8.895 degrees, +/−8.896 degrees, +/−8.897 degrees, +/−8.898 degrees, +/−8.899 degrees, +/−8.900 degrees, degrees, +/−8.901 degrees, +/−8.902 degrees, +/−8.903 degrees, +/−8.904 degrees, +/−8.905 degrees, +/−8.906 degrees, +/−8.907 degrees, +/−8.908 degrees, +/−8.909 degrees, +/−8.910 degrees, +/−8.911 degrees, +/−8.912 degrees, +/−8.913 degrees, +/−8.914 degrees, +/−8.915 degrees, +/−8.916 degrees, +/−8.917 degrees, +/−8.918 degrees, +/−8.919 degrees, +/−8.920 degrees, +/−8.921 degrees, +/−8.922 degrees, +/−8.923 degrees, +/−8.924 degrees, +/−8.925 degrees, +/−8.926 degrees, +/−8.927 degrees, +/−8.928 degrees, +/−8.929 degrees, +/−8.930 degrees, +/−8.931 degrees, +/−8.932 degrees, +/−8.933 degrees, +/−8.934 degrees, +/−8.935 degrees, +/−8.936 degrees, +/−8.937 degrees, +/−8.938 degrees, +/−8.939 degrees, +/−8.940 degrees, +/−8.941 degrees, +/−8.942 degrees, +/−8.943 degrees, +/−8.944 degrees, +/−8.945 degrees, +/−8.946 degrees, +/−8.947 degrees, +/−8.948 degrees, +/−8.949 degrees, +/−8.950 degrees, +/−8.951 degrees, +/−8.952 degrees, +/−8.953 degrees, +/−8.954 degrees, +/−8.955 degrees, +/−8.956 degrees, +/−8.957 degrees, +/−8.958 degrees, +/−8.959 degrees, +/−8.960 degrees, +/−8.961 degrees, +/−8.962 degrees, +/−8.963 degrees, +/−8.964 degrees, +/−8.965 degrees, +/−8.966 degrees, +/−8.967 degrees, +/−8.968 degrees, +/−8.969 degrees, +/−8.970 degrees, +/−8.971 degrees, +/−8.972 degrees, +/−8.973 degrees, +/−8.974 degrees, +/−8.975 degrees, +/−8.976 degrees, +/−8.977 degrees, +/−8.978 degrees, +/−8.979 degrees, +/−8.980 degrees, +/−8.981 degrees, +/−8.982 degrees, +/−8.983 degrees, +/−8.984 degrees, +/−8.985 degrees, +/−8.986 degrees, +/−8.987 degrees, +/−8.988 degrees, +/−8.989 degrees, +/−8.990 degrees, +/−8.991 degrees, +/−8.992 degrees, +/−8.993 degrees, +/−8.994 degrees, +/−8.995 degrees, +/−8.996 degrees, +/−8.997 degrees, +/−8.998 degrees, +/−8.999 degrees, +/−9.000 degrees, +/−9.100, degrees, +/−9.101 degrees, +/−9.102 degrees, +/−9.103 degrees, +/−9.104 degrees, +/−9.105 degrees, +/−9.106 degrees, +/−9.107 degrees, +/−9.108 degrees, +/−9.109 degrees, +/−9.110 degrees, +/−9.111 degrees, +/−9.112 degrees, +/−9.113 degrees, +/−9.114 degrees, +/−9.115 degrees, +/−9.116 degrees, +/−9.117 degrees, +/−9.118 degrees, +/−9.119 degrees, +/−9.120 degrees, +/−9.121 degrees, +/−9.122 degrees, +/−9.123 degrees, +/−9.124 degrees, +/−9.125 degrees, +/−9.126 degrees, +/−9.127 degrees, +/−9.128 degrees, +/−9.129 degrees, +/−9.130 degrees, +/−9.131 degrees, +/−9.132 degrees, +/−9.133 degrees, +/−9.134 degrees, +/−9.135 degrees, +/−9.136 degrees, +/−9.137 degrees, +/−9.138 degrees, +/−9.139 degrees, +/−9.140 degrees, +/−9.141 degrees, +/−9.142 degrees, +/−9.143 degrees, +/−9.144 degrees, +/−9.145 degrees, +/−9.146 degrees, +/−9.147 degrees, +/−9.148 degrees, +/−9.149 degrees, +/−9.150 degrees, +/−9.151 degrees, +/−9.152 degrees, +/−9.153 degrees, +/−9.154 degrees, +/−9.155 degrees, +/−9.156 degrees, +/−9.157 degrees, +/−9.158 degrees, +/−9.159 degrees, +/−9.160 degrees, +/−9.161 degrees, +/−9.162 degrees, +/−9.163 degrees, +/−9.164 degrees, +/−9.165 degrees, +/−9.166 degrees, +/−9.167 degrees, +/−9.168 degrees, +/−9.169 degrees, +/−9.170 degrees, +/−9.171 degrees, +/−9.172 degrees, +/−9.173 degrees, +/−9.174 degrees, +/−9.175 degrees, +/−9.176 degrees, +/−9.177 degrees, +/−9.178 degrees, +/−9.179 degrees, +/−9.180 degrees, +/−9.181 degrees, +/−9.182 degrees, +/−9.183 degrees, +/−9.184 degrees, +/−9.185 degrees, +/−9.186 degrees, +/−9.187 degrees, +/−9.188 degrees, +/−9.189 degrees, +/−9.190 degrees, +/−9.191 degrees, +/−9.192 degrees, +/−9.193 degrees, +/−9.194 degrees, +/−9.195 degrees, +/−9.196 degrees, +/−9.197 degrees, +/−9.198 degrees, +/−9.199 degrees, +/−9.200 degrees, degrees, +/−9.201 degrees, +/−9.202 degrees, +/−9.203 degrees, +/−9.204 degrees, +/−9.205 degrees, +/−9.206 degrees, +/−9.207 degrees, +/−9.208 degrees, +/−9.209 degrees, +/−9.210 degrees, +/−9.211 degrees, +/−9.212 degrees, +/−9.213 degrees, +/−9.214 degrees, +/−9.215 degrees, +/−9.216 degrees, +/−9.217 degrees, +/−9.218 degrees, +/−9.219 degrees, +/−9.220 degrees, +/−9.221 degrees, +/−9.222 degrees, +/−9.223 degrees, +/−9.224 degrees, +/−9.225 degrees, +/−9.226 degrees, +/−9.227 degrees, +/−9.228 degrees, +/−9.229 degrees, +/−9.230 degrees, +/−9.231 degrees, +/−9.232 degrees, +/−9.233 degrees, +/−9.234 degrees, +/−9.235 degrees, +/−9.236 degrees, +/−9.237 degrees, +/−9.238 degrees, +/−9.239 degrees, +/−9.240 degrees, +/−9.241 degrees, +/−9.242 degrees, +/−9.243 degrees, +/−9.244 degrees, +/−9.245 degrees, +/−9.246 degrees, +/−9.247 degrees, +/−9.248 degrees, +/−9.249 degrees, +/−9.250 degrees, +/−9.251 degrees, +/−9.252 degrees, +/−9.253 degrees, +/−9.254 degrees, +/−9.255 degrees, +/−9.256 degrees, +/−9.257 degrees, +/−9.258 degrees, +/−9.259 degrees, +/−9.260 degrees, +/−9.261 degrees, +/−9.262 degrees, +/−9.263 degrees, +/−9.264 degrees, +/−9.265 degrees, +/−9.266 degrees, +/−9.267 degrees, +/−9.268 degrees, +/−9.269 degrees, +/−9.270 degrees, +/−9.271 degrees, +/−9.272 degrees, +/−9.273 degrees, +/−9.274 degrees, +/−9.275 degrees, +/−9.276 degrees, +/−9.277 degrees, +/−9.278 degrees, +/−9.279 degrees, +/−9.280 degrees, +/−9.281 degrees, +/−9.282 degrees, +/−9.283 degrees, +/−9.284 degrees, +/−9.285 degrees, +/−9.286 degrees, +/−9.287 degrees, +/−9.288 degrees, +/−9.289 degrees, +/−9.290 degrees, +/−9.291 degrees, +/−9.292 degrees, +/−9.293 degrees, +/−9.294 degrees, +/−9.295 degrees, +/−9.296 degrees, +/−9.297 degrees, +/−9.298 degrees, +/−9.299 degrees, +/−9.300 degrees, degrees, +/−9.301 degrees, +/−9.302 degrees, +/−9.303 degrees, +/−9.304 degrees, +/−9.305 degrees, +/−9.306 degrees, +/−9.307 degrees, +/−9.308 degrees, +/−9.309 degrees, +/−9.310 degrees, +/−9.311 degrees, +/−9.312 degrees, +/−9.313 degrees, +/−9.314 degrees, +/−9.315 degrees, +/−9.316 degrees, +/−9.317 degrees, +/−9.318 degrees, +/−9.319 degrees, +/−9.320 degrees, +/−9.321 degrees, +/−9.322 degrees, +/−9.323 degrees, +/−9.324 degrees, +/−9.325 degrees, +/−9.326 degrees, +/−9.327 degrees, +/−9.328 degrees, +/−9.329 degrees, +/−9.330 degrees, +/−9.331 degrees, +/−9.332 degrees, +/−9.333 degrees, +/−9.334 degrees, +/−9.335 degrees, +/−9.336 degrees, +/−9.337 degrees, +/−9.338 degrees, +/−9.339 degrees, +/−9.340 degrees, +/−9.341 degrees, +/−9.342 degrees, +/−9.343 degrees, +/−9.344 degrees, +/−9.345 degrees, +/−9.346 degrees, +/−9.347 degrees, +/−9.348 degrees, +/−9.349 degrees, +/−9.350 degrees, +/−9.351 degrees, +/−9.352 degrees, +/−9.353 degrees, +/−9.354 degrees, +/−9.355 degrees, +/−9.356 degrees, +/−9.357 degrees, +/−9.358 degrees, +/−9.359 degrees, +/−9.360 degrees, +/−9.361 degrees, +/−9.362 degrees, +/−9.363 degrees, +/−9.364 degrees, +/−9.365 degrees, +/−9.366 degrees, +/−9.367 degrees, +/−9.368 degrees, +/−9.369 degrees, +/−9.370 degrees, +/−9.371 degrees, +/−9.372 degrees, +/−9.373 degrees, +/−9.374 degrees, +/−9.375 degrees, +/−9.376 degrees, +/−9.377 degrees, +/−9.378 degrees, +/−9.379 degrees, +/−9.380 degrees, +/−9.381 degrees, +/−9.382 degrees, +/−9.383 degrees, +/−9.384 degrees, +/−9.385 degrees, +/−9.386 degrees, +/−9.387 degrees, +/−9.388 degrees, +/−9.389 degrees, +/−9.390 degrees, +/−9.391 degrees, +/−9.392 degrees, +/−9.393 degrees, +/−9.394 degrees, +/−9.395 degrees, +/−9.396 degrees, +/−9.397 degrees, +/−9.398 degrees, +/−9.399 degrees, +/−9.400 degrees, degrees, +/−9.401 degrees, +/−9.402 degrees, +/−9.403 degrees, +/−9.404 degrees, +/−9.405 degrees, +/−9.406 degrees, +/−9.407 degrees, +/−9.408 degrees, +/−9.409 degrees, +/−9.410 degrees, +/−9.411 degrees, +/−9.412 degrees, +/−9.413 degrees, +/−9.414 degrees, +/−9.415 degrees, +/−9.416 degrees, +/−9.417 degrees, +/−9.418 degrees, +/−9.419 degrees, +/−9.420 degrees, +/−9.421 degrees, +/−9.422 degrees, +/−9.423 degrees, +/−9.424 degrees, +/−9.425 degrees, +/−9.426 degrees, +/−9.427 degrees, +/−9.428 degrees, +/−9.429 degrees, +/−9.430 degrees, +/−9.431 degrees, +/−9.432 degrees, +/−9.433 degrees, +/−9.434 degrees, +/−9.435 degrees, +/−9.436 degrees, +/−9.437 degrees, +/−9.438 degrees, +/−9.439 degrees, +/−9.440 degrees, +/−9.441 degrees, +/−9.442 degrees, +/−9.443 degrees, +/−9.444 degrees, +/−9.445 degrees, +/−9.446 degrees, +/−9.447 degrees, +/−9.448 degrees, +/−9.449 degrees, +/−9.450 degrees, +/−9.451 degrees, +/−9.452 degrees, +/−9.453 degrees, +/−9.454 degrees, +/−9.455 degrees, +/−9.456 degrees, +/−9.457 degrees, +/−9.458 degrees, +/−9.459 degrees, +/−9.460 degrees, +/−9.461 degrees, +/−9.462 degrees, +/−9.463 degrees, +/−9.464 degrees, +/−9.465 degrees, +/−9.466 degrees, +/−9.467 degrees, +/−9.468 degrees, +/−9.469 degrees, +/−9.470 degrees, +/−9.471 degrees, +/−9.472 degrees, +/−9.473 degrees, +/−9.474 degrees, +/−9.475 degrees, +/−9.476 degrees, +/−9.477 degrees, +/−9.478 degrees, +/−9.479 degrees, +/−9.480 degrees, +/−9.481 degrees, +/−9.482 degrees, +/−9.483 degrees, +/−9.484 degrees, +/−9.485 degrees, +/−9.486 degrees, +/−9.487 degrees, +/−9.488 degrees, +/−9.489 degrees, +/−9.490 degrees, +/−9.491 degrees, +/−9.492 degrees, +/−9.493 degrees, +/−9.494 degrees, +/−9.495 degrees, +/−9.496 degrees, +/−9.497 degrees, +/−9.498 degrees, +/−9.499 degrees, +/−9.500 degrees, degrees, +/−9.501 degrees, +/−9.502 degrees, +/−9.503 degrees, +/−9.504 degrees, +/−9.505 degrees, +/−9.506 degrees, +/−9.507 degrees, +/−9.508 degrees, +/−9.509 degrees, +/−9.510 degrees, +/−9.511 degrees, +/−9.512 degrees, +/−9.513 degrees, +/−9.514 degrees, +/−9.515 degrees, +/−9.516 degrees, +/−9.517 degrees, +/−9.518 degrees, +/−9.519 degrees, +/−9.520 degrees, +/−9.521 degrees, +/−9.522 degrees, +/−9.523 degrees, +/−9.524 degrees, +/−9.525 degrees, +/−9.526 degrees, +/−9.527 degrees, +/−9.528 degrees, +/−9.529 degrees, +/−9.530 degrees, +/−9.531 degrees, +/−9.532 degrees, +/−9.533 degrees, +/−9.534 degrees, +/−9.535 degrees, +/−9.536 degrees, +/−9.537 degrees, +/−9.538 degrees, +/−9.539 degrees, +/−9.540 degrees, +/−9.541 degrees, +/−9.542 degrees, +/−9.543 degrees, +/−9.544 degrees, +/−9.545 degrees, +/−9.546 degrees, +/−9.547 degrees, +/−9.548 degrees, +/−9.549 degrees, +/−9.550 degrees, +/−9.551 degrees, +/−9.552 degrees, +/−9.553 degrees, +/−9.554 degrees, +/−9.555 degrees, +/−9.556 degrees, +/−9.557 degrees, +/−9.558 degrees, +/−9.559 degrees, +/−9.560 degrees, +/−9.561 degrees, +/−9.562 degrees, +/−9.563 degrees, +/−9.564 degrees, +/−9.565 degrees, +/−9.566 degrees, +/−9.567 degrees, +/−9.568 degrees, +/−9.569 degrees, +/−9.570 degrees, +/−9.571 degrees, +/−9.572 degrees, +/−9.573 degrees, +/−9.574 degrees, +/−9.575 degrees, +/−9.576 degrees, +/−9.577 degrees, +/−9.578 degrees, +/−9.579 degrees, +/−9.580 degrees, +/−9.581 degrees, +/−9.582 degrees, +/−9.583 degrees, +/−9.584 degrees, +/−9.585 degrees, +/−9.586 degrees, +/−9.587 degrees, +/−9.588 degrees, +/−9.589 degrees, +/−9.590 degrees, +/−9.591 degrees, +/−9.592 degrees, +/−9.593 degrees, +/−9.594 degrees, +/−9.595 degrees, +/−9.596 degrees, +/−9.597 degrees, +/−9.598 degrees, +/−9.599 degrees, +/−9.600 degrees, degrees, +/−9.601 degrees, +/−9.602 degrees, +/−9.603 degrees, +/−9.604 degrees, +/−9.605 degrees, +/−9.606 degrees, +/−9.607 degrees, +/−9.608 degrees, +/−9.609 degrees, +/−9.610 degrees, +/−9.611 degrees, +/−9.612 degrees, +/−9.613 degrees, +/−9.614 degrees, +/−9.615 degrees, +/−9.616 degrees, +/−9.617 degrees, +/−9.618 degrees, +/−9.619 degrees, +/−9.620 degrees, +/−9.621 degrees, +/−9.622 degrees, +/−9.623 degrees, +/−9.624 degrees, +/−9.625 degrees, +/−9.626 degrees, +/−9.627 degrees, +/−9.628 degrees, +/−9.629 degrees, +/−9.630 degrees, +/−9.631 degrees, +/−9.632 degrees, +/−9.633 degrees, +/−9.634 degrees, +/−9.635 degrees, +/−9.636 degrees, +/−9.637 degrees, +/−9.638 degrees, +/−9.639 degrees, +/−9.640 degrees, +/−9.641 degrees, +/−9.642 degrees, +/−9.643 degrees, +/−9.644 degrees, +/−9.645 degrees, +/−9.646 degrees, +/−9.647 degrees, +/−9.648 degrees, +/−9.649 degrees, +/−9.650 degrees, +/−9.651 degrees, +/−9.652 degrees, +/−9.653 degrees, +/−9.654 degrees, +/−9.655 degrees, +/−9.656 degrees, +/−9.657 degrees, +/−9.658 degrees, +/−9.659 degrees, +/−9.660 degrees, +/−9.661 degrees, +/−9.662 degrees, +/−9.663 degrees, +/−9.664 degrees, +/−9.665 degrees, +/−9.666 degrees, +/−9.667 degrees, +/−9.668 degrees, +/−9.669 degrees, +/−9.670 degrees, +/−9.671 degrees, +/−9.672 degrees, +/−9.673 degrees, +/−9.674 degrees, +/−9.675 degrees, +/−9.676 degrees, +/−9.677 degrees, +/−9.678 degrees, +/−9.679 degrees, +/−9.680 degrees, +/−9.681 degrees, +/−9.682 degrees, +/−9.683 degrees, +/−9.684 degrees, +/−9.685 degrees, +/−9.686 degrees, +/−9.687 degrees, +/−9.688 degrees, +/−9.689 degrees, +/−9.690 degrees, +/−9.691 degrees, +/−9.692 degrees, +/−9.693 degrees, +/−9.694 degrees, +/−9.695 degrees, +/−9.696 degrees, +/−9.697 degrees, +/−9.698 degrees, +/−9.699 degrees, +/−9.700 degrees, degrees, +/−9.701 degrees, +/−9.702 degrees, +/−9.703 degrees, +/−9.704 degrees, +/−9.705 degrees, +/−9.706 degrees, +/−9.707 degrees, +/−9.708 degrees, +/−9.709 degrees, +/−9.710 degrees, +/−9.711 degrees, +/−9.712 degrees, +/−9.713 degrees, +/−9.714 degrees, +/−9.715 degrees, +/−9.716 degrees, +/−9.717 degrees, +/−9.718 degrees, +/−9.719 degrees, +/−9.720 degrees, +/−9.721 degrees, +/−9.722 degrees, +/−9.723 degrees, +/−9.724 degrees, +/−9.725 degrees, +/−9.726 degrees, +/−9.727 degrees, +/−9.728 degrees, +/−9.729 degrees, +/−9.730 degrees, +/−9.731 degrees, +/−9.732 degrees, +/−9.733 degrees, +/−9.734 degrees, +/−9.735 degrees, +/−9.736 degrees, +/−9.737 degrees, +/−9.738 degrees, +/−9.739 degrees, +/−9.740 degrees, +/−9.741 degrees, +/−9.742 degrees, +/−9.743 degrees, +/−9.744 degrees, +/−9.745 degrees, +/−9.746 degrees, +/−9.747 degrees, +/−9.748 degrees, +/−9.749 degrees, +/−9.750 degrees, +/−9.751 degrees, +/−9.752 degrees, +/−9.753 degrees, +/−9.754 degrees, +/−9.755 degrees, +/−9.756 degrees, +/−9.757 degrees, +/−9.758 degrees, +/−9.759 degrees, +/−9.760 degrees, +/−9.761 degrees, +/−9.762 degrees, +/−9.763 degrees, +/−9.764 degrees, +/−9.765 degrees, +/−9.766 degrees, +/−9.767 degrees, +/−9.768 degrees, +/−9.769 degrees, +/−9.770 degrees, +/−9.771 degrees, +/−9.772 degrees, +/−9.773 degrees, +/−9.774 degrees, +/−9.775 degrees, +/−9.776 degrees, +/−9.777 degrees, +/−9.778 degrees, +/−9.779 degrees, +/−9.780 degrees, +/−9.781 degrees, +/−9.782 degrees, +/−9.783 degrees, +/−9.784 degrees, +/−9.785 degrees, +/−9.786 degrees, +/−9.787 degrees, +/−9.788 degrees, +/−9.789 degrees, +/−9.790 degrees, +/−9.791 degrees, +/−9.792 degrees, +/−9.793 degrees, +/−9.794 degrees, +/−9.795 degrees, +/−9.796 degrees, +/−9.797 degrees, +/−9.798 degrees, +/−9.799 degrees, +/−9.800 degrees, degrees, +/−9.801 degrees, +/−9.802 degrees, +/−9.803 degrees, +/−9.804 degrees, +/−9.805 degrees, +/−9.806 degrees, +/−9.807 degrees, +/−9.808 degrees, +/−9.809 degrees, +/−9.810 degrees, +/−9.811 degrees, +/−9.812 degrees, +/−9.813 degrees, +/−9.814 degrees, +/−9.815 degrees, +/−9.816 degrees, +/−9.817 degrees, +/−9.818 degrees, +/−9.819 degrees, +/−9.820 degrees, +/−9.821 degrees, +/−9.822 degrees, +/−9.823 degrees, +/−9.824 degrees, +/−9.825 degrees, +/−9.826 degrees, +/−9.827 degrees, +/−9.828 degrees, +/−9.829 degrees, +/−9.830 degrees, +/−9.831 degrees, +/−9.832 degrees, +/−9.833 degrees, +/−9.834 degrees, +/−9.835 degrees, +/−9.836 degrees, +/−9.837 degrees, +/−9.838 degrees, +/−9.839 degrees, +/−9.840 degrees, +/−9.841 degrees, +/−9.842 degrees, +/−9.843 degrees, +/−9.844 degrees, +/−9.845 degrees, +/−9.846 degrees, +/−9.847 degrees, +/−9.848 degrees, +/−9.849 degrees, +/−9.850 degrees, +/−9.851 degrees, +/−9.852 degrees, +/−9.853 degrees, +/−9.854 degrees, +/−9.855 degrees, +/−9.856 degrees, +/−9.857 degrees, +/−9.858 degrees, +/−9.859 degrees, +/−9.860 degrees, +/−9.861 degrees, +/−9.862 degrees, +/−9.863 degrees, +/−9.864 degrees, +/−9.865 degrees, +/−9.866 degrees, +/−9.867 degrees, +/−9.868 degrees, +/−9.869 degrees, +/−9.870 degrees, +/−9.871 degrees, +/−9.872 degrees, +/−9.873 degrees, +/−9.874 degrees, +/−9.875 degrees, +/−9.876 degrees, +/−9.877 degrees, +/−9.878 degrees, +/−9.879 degrees, +/−9.880 degrees, +/−9.881 degrees, +/−9.882 degrees, +/−9.883 degrees, +/−9.884 degrees, +/−9.885 degrees, +/−9.886 degrees, +/−9.887 degrees, +/−9.888 degrees, +/−9.889 degrees, +/−9.890 degrees, +/−9.891 degrees, +/−9.892 degrees, +/−9.893 degrees, +/−9.894 degrees, +/−9.895 degrees, +/−9.896 degrees, +/−9.897 degrees, +/−9.898 degrees, +/−9.899 degrees, +/−9.900 degrees, degrees, +/−9.901 degrees, +/−9.902 degrees, +/−9.903 degrees, +/−9.904 degrees, +/−9.905 degrees, +/−9.906 degrees, +/−9.907 degrees, +/−9.908 degrees, +/−9.909 degrees, +/−9.910 degrees, +/−9.911 degrees, +/−9.912 degrees, +/−9.913 degrees, +/−9.914 degrees, +/−9.915 degrees, +/−9.916 degrees, +/−9.917 degrees, +/−9.918 degrees, +/−9.919 degrees, +/−9.920 degrees, +/−9.921 degrees, +/−9.922 degrees, +/−9.923 degrees, +/−9.924 degrees, +/−9.925 degrees, +/−9.926 degrees, +/−9.927 degrees, +/−9.928 degrees, +/−9.929 degrees, +/−9.930 degrees, +/−9.931 degrees, +/−9.932 degrees, +/−9.933 degrees, +/−9.934 degrees, +/−9.935 degrees, +/−9.936 degrees, +/−9.937 degrees, +/−9.938 degrees, +/−9.939 degrees, +/−9.940 degrees, +/−9.941 degrees, +/−9.942 degrees, +/−9.943 degrees, +/−9.944 degrees, +/−9.945 degrees, +/−9.946 degrees, +/−9.947 degrees, +/−9.948 degrees, +/−9.949 degrees, +/−9.950 degrees, +/−9.951 degrees, +/−9.952 degrees, +/−9.953 degrees, +/−9.954 degrees, +/−9.955 degrees, +/−9.956 degrees, +/−9.957 degrees, +/−9.958 degrees, +/−9.959 degrees, +/−9.960 degrees, +/−9.961 degrees, +/−9.962 degrees, +/−9.963 degrees, +/−9.964 degrees, +/−9.965 degrees, +/−9.966 degrees, +/−9.967 degrees, +/−9.968 degrees, +/−9.969 degrees, +/−9.970 degrees, +/−9.971 degrees, +/−9.972 degrees, +/−9.973 degrees, +/−9.974 degrees, +/−9.975 degrees, +/−9.976 degrees, +/−9.977 degrees, +/−9.978 degrees, +/−9.979 degrees, +/−9.980 degrees, +/−9.981 degrees, +/−9.982 degrees, +/−9.983 degrees, +/−9.984 degrees, +/−9.985 degrees, +/−9.986 degrees, +/−9.987 degrees, +/−9.988 degrees, +/−9.989 degrees, +/−9.990 degrees, +/−9.991 degrees, +/−9.992 degrees, +/−9.993 degrees, +/−9.994 degrees, +/−9.995 degrees, +/−9.996 degrees, +/−9.997 degrees, +/−9.998 degrees, +/−9.999 degrees, +/−10.000 degrees, . . . , and 20.001 degrees.

In various examples, the system, method, and/or device may implement one or more of skin and/or body temperature changes, body temperature changes, weight loss action changes, calorie utilization changes, and/or any other actions disclosed herein based on one or more sensor data, program mode, input by the person, programmed functions, and/or any other condition including functional.

In one set of examples, the target temperature range may be from +/−.1 degrees, +/−0.5 degrees and/or to +/−1.0 degree (and/or anywhere in between—which for brevity are not listed). In another set of examples, the target temperature range may be from +/−1.2 degrees, +/−1.6 degrees, and/or to +/−2.001 degrees (and/or anywhere in between—which for brevity are not listed). In another set of examples, the target temperature range may be from +/−2.312 degrees, +/−2.4 degrees, and/or to +/−2.6 degrees (and/or anywhere in between—which for brevity are not listed). In another set of examples, the target temperature range may be from +/−0.1 to 2.0 degrees (and/or anywhere in between—which for brevity are not listed). In another set of examples, the target temperature range may be from 0 to +/−1.0 degree (and/or anywhere in between—which for brevity are not listed). In another set of examples, the target temperature range may be from +/−2.7 degrees, +/−3.15 degrees, and/or to +/−3.7 degrees (and/or anywhere in between—which for brevity are not listed). In another set of examples, the target temperature range may be from +/−3.333 degrees, +/−3.75 degrees, and/or to +/−4.0 degrees (and/or anywhere in between—which for brevity are not listed). In another set of examples, the target temperature range may be from +/−3.9 degrees, +/−4.0 degrees, and/or to +/−4.5 degrees (and/or anywhere in between—which for brevity are not listed). In another set of examples, the target temperature range may be from +/−5.0 degrees, +/−7.5 degrees, and/or to +/−20.0 degrees (and/or anywhere in between—which for brevity are not listed).

In one embodiment, an article of clothing includes an exterior surface; an interior surface; one or more tubes attached to the interior surface; a heat exchanger coupled to the one or more tubes; and a power source. In another embodiment, an article of clothing includes a shirt with one or more tubes attached to the shirt; at least one of a tube and a balloon attached the shirt; a reservoir and a heat exchanged coupled to the at least one of the tube and the balloon; and a power source. In another embodiment, an article of clothing includes a shirt with one or more tubes attached to the shirt; at least one of a tube and a balloon attached the shirt; a reservoir and a heat exchanged coupled to the at least one of the tube and the balloon; a power source; and a transceiver. In another embodiment, a tracking device includes a camera; a processor; and a memory. In another embodiment, an article of clothing includes an exterior surface; an interior surface; one or more Peltier devices; and a power source.

1. A computing device comprising:
one or more processors configured to receive at least a first image and a second image, the one or more processors configured to determine a starting calories content of a first consumable item from extracting data from the first image and an ending calories content of the first consumable item from extracting data from the second image, the one or more processors configured to determine a first consumed calories by calculating a difference between the starting calories content of the first consumable item and the ending calories content of the first consumable item.

2. The computing device of claim 1:
wherein the one or more processors utilize at least one of: one or more engines; one or more lookup tables; one or more libraries; an Internet; a calorie counter program; user default settings; system default settings; a bar code; one or more scanned words; crowd sourced data; one or more machine learning systems; one or more voice commands, and one or more content identifiers to determine the starting calories content of the first consumable item and the ending calories content of the first consumable item.

3. The computing device of claim 1:
wherein the one or more processors are further configured to determine at least one of: a protein content; a sugar content; a vegetable content; a carbohydrate content; a vitamin content; and a nutritional value of at least one of the starting calories content of the first consumable item; the ending calories content of the first consumable item; and the first consumed calories.

4. The computing device of claim 1 further comprising:
a display;
wherein the one or more processors are configured to compare the first consumed calories to a calorie target and generate a first report on the display relating to the comparison of the first consumed calories to the calorie target.

5. The computing device of claim 4:
wherein the one or more processors are configured to generate at least one of: one or more warnings; one or more recommendations; one or more promotional offers; one or more messages; and commands to initiate one or more actions based on the comparison of the first consumed calories to the calorie target.

6. The computing device of claim 1 further comprising:
wherein the one or more processors are configured to receive at least a third image and a fourth image, the one or more processors are configured to determine a starting calories content of a second consumable item from extracting data from the third image and an ending calories content of the second consumable item from extracting data from the fourth image, the one or more processors are configured to determine a second consumed calories by calculating a difference between the starting calories content of the second consumable item and the ending calories content of the second consumable item.

7. The computing device of claim 6 further comprising:
a display;
wherein the one or more processors are configured to compare the second consumed calories to a calorie target and generate a second report on the display relating to the comparison of the second consumed calories to the calorie target.

8. The computing device of claim 7:
wherein the one or more processors are configured to generate at least one of: one or more warnings; one or more recommendations; one or more promotional offers; one or more messages; and commands to initiate one or more actions based on the comparison of the second consumed calories to the calorie target.

9. The computing device of claim 1 further comprising:
wherein the one or more processors are configured to receive at least an Nth−1 image and an Nth image, the one or more processors are configured to determine a starting calories content of an Nth consumable item from extracting data from the Nth−1 image and an ending calories content of the Nth consumable item from extracting data from the Nth image, the one or more processors are configured to determine an Nth consumed calories by calculating a difference between the starting calories content of the Nth consumable item and the ending calories content of the Nth consumable item.

10. The computing device of claim 9 further comprising:
a display;
wherein the one or more processors are configured to compare the Nth consumed calories to a calorie target and generate an Nth report on the display relating to the comparison of the Nth consumed calories to the calorie target.

11. The computing device of claim 10:
wherein the one or more processors are configured to generate at least one of: one or more warnings; one or more recommendations; one or more promotional offers; one or more messages; and commands to initiate one or more actions based on the comparison of the Nth consumed calories to the calorie target.

12. The computing device of claim 1 further comprising:
a display;
wherein the one or more processors are configured to receive at least a third image and a fourth image, the one or more processors are configured to determine a starting calories content of a second consumable item from extracting data from the third image and an ending calories content of the second consumable item from extracting data from the fourth image, the one or more processors are configured to determine a second consumed calories by calculating a difference between the starting calories content of the second consumable item and the ending calories content of the second consumable item;
wherein the one or more processors are configured to receive at least an Nth−1 image and an Nth image, the one or more processors are configured to determine a starting calories content of an Nth consumable item from extracting data from the Nth−1 image and an ending calories content of the Nth consumable item from extracting data from the Nth image, the one or more processors are configured to determine an Nth consumed calories by calculating a difference between the starting calories content of the Nth consumable item and the ending calories content of the Nth consumable item;
wherein the one or more processors are configured to compare the second consumed calories to a calorie target and generate a second report on the display relating to the comparison of the second consumed calories to the calorie target;
wherein the one or more processors are configured to compare a total of the first consumed calories, the second consumed calories, and the Nth consumed calories to a calorie target and generate a consumed calorie report on the display relating to the comparison of the first consumed calories, the second consumed calories, and the Nth consumed calories to the calorie target.

13. The computing device of claim 1:
wherein the one or more processors are configured to complete one or more of the following items: determine one or more consumed medicines; determine one or more completed exercises; receive one or more body signals; and generate one or more warnings; one or more recommendations; one or more promotional offers; one or more messages; and initiate one or more actions based on the comparison of the first consumed calories, the second consumed calories, and the Nth consumed calories to the calorie target.

14. The computing device of claim 1:
wherein the one or more processors are configured to complete one or more of the following items: determine one or more consumed medicines; determine one or more completed exercises; and receive one or more body signals; and
the one or more processors configured to generate at least one of: one or more warnings; one or more recommendations; one or more promotional offers; one or more messages; and commands to initiate one or more actions based on at least one of the determined medicines consumed; determined exercises completed; and received one or more body signals.

15. A computing device comprising:
one or more processors configured to receive at least a first image, the one or more processors configured to determine a starting calories content of a first consumable item from extracting data from the first image, the one or more processors configured to determine a contents of the first consumable item, and the one or more processors configured to determine a consumed medicine or a user's health condition.

16. The computing device of claim 15:
wherein the one or more processors are configured to generate at least one of: one or more warnings; one or more recommendations; one or more messages; and commands to initiate one or more actions based on at least one of: a comparison of the contents of the first consumable item to at least one of the consumed medicine and the user's health condition; and a comparison of the starting calories content of the first consumable item to the consumed medicine or the user's health condition.

17. The computing device of claim 15:
wherein the one or more processors are configured to receive one or more body signals, the one or more processors configured to generate at least one of: one or more warnings; one or more recommendations; one or more messages; and commands to initiate one or more actions based on the one or more body signals.

18. The computing device of claim 17:
wherein the one or more body signals indicates at least one of: an increase in blood pressure; a decrease in blood pressure; an increase in heart rate; a decrease in heart rate; an increase in blood sugar level; a decrease in blood sugar level; an increase in a bodily function; and a decrease in a bodily function.

19. A computing device comprising:
one or more processors configured to:
receive at least a first image and a second image, the one or more processors configured to determine a starting calories content of a first consumable item from extracting data from the first image and an ending calories content of the first consumable item from extracting data from the second image, the one or more processors configured to determine a first consumed calories by calculating a difference between the starting calories content of the first consumable item and the ending calories content of the first consumable item, the one or more processors configured to add the first consumed calories to a calorie count;
receive data relating to a first exercise completed with a first utilized calories, the one or more processors configured to substrate the first utilized calories from the calorie count;
receive at least a third image and a fourth image, the one or more processors are configured to determine a starting calories content of a second consumable item from extracting data from the third image and an ending calories content of the second consumable item from extracting data from the fourth image, the one or more processors are configured to determine a second consumed calories by calculating a difference between the starting calories content of the second consumable item and the ending calories content of the second consumable item, the one or more processors configured to add the second consumed calories to the calorie count;
receive data relating to an Nth exercise completed with an Nth utilized calories, the one or more processors configured to substrate the Nth utilized calories from the calorie count;
receive at least an Nth−1 image and an Nth image, the one or more processors are configured to determine a starting calories content of an Nth consumable item from extracting data from the Nth−1 image and an ending calories content of the Nth consumable item from extracting data from the Nth image, the one or more processors are configured to determine an Nth consumed calories by calculating a difference between the starting calories content of the Nth consumable item and the ending calories content of the Nth consumable item, the one or more processors configured to add the Nth consumed calories to the calorie count; and
generate one or more reports based on at least one of: the first consumed calories, the second consumed calories, the Nth consumed calories, the first utilized calories, the Nth utilized calories, and the calorie count.

20. The computing device of claim 19:
wherein the one or more processors are configured to generate at least one of: one or more warnings; one or more recommendations; one or more messages; and commands to initiate one or more actions based on the first consumed calories, the second consumed calories, the Nth consumed calories, the first utilized calories, the Nth utilized calories, and the calorie count.
